(19) United States
(12) Patent Application Publication
(10) Pub. No.: US 2022/0049224 A1
(43) Pub. Date: Feb. 17, 2022

(54) CULTURE MODIFICATIONS OF STEM CELLS TO ENHANCE THERAPEUTIC RECOVERY FROM TISSUE DAMAGE AND ISCHEMIA-REPERFUSION INJURY AND DELAYED ORGAN FUNCTION

(71) Applicant: Board of Supervisors of Louisiana State University and Agricultural and Mechanical College, Baton Rouge, LA (US)

(72) Inventors: Jonathan ALEXANDER, Shreveport, LA (US); Mansoureh BARZEGAR, Shreveport, LA (US); Yuping WANG, Shreveport, LA (US)

(21) Appl. No.: 17/086,315

(22) Filed: Oct. 30, 2020

Related U.S. Application Data
(60) Provisional application No. 62/928,333, filed on Oct. 30, 2019.

(51) Int. Cl. C12N 5/0775; A61K 35/28

(52) U.S. Cl. CPC C12N 5/0668; C12N 2500/36; A61K 35/28

(57) ABSTRACT

Therapeutics comprising Cells and methods of creating enhanced human Placenta Mesenchymal Stem Cells (hPMSCs) comprising culturing the hPMSCs with cholesterol for one of between 24 and 96 hours and between 48 and 72 hours. Therapeutics comprising microparticles and methods of creating enhanced microparticles from hPMSCs comprising culturing the hPMSCs with cholesterol for one of between 24 and 96 hours and between 48 and 72 hours, and then separating the enhanced microparticles from the hPMSCs. Therapeutics comprising exosomes and methods of creating enhanced exosomes from hPMSCs comprising culturing the hPMSCs with cholesterol for one of between 24 and 96 hours and between 48 and 72 hours, and then separating the enhanced microparticles from the hPMSCs. Therapeutic comprising extracellular vesicles and methods of creating enhanced EVs from hPMSCs comprising culturing the hPMSCs with cholesterol for one of between 24 and 96 hours and between 48 and 72 hours.

### CROSS REFERENCE TO RELATED APPLICATIONS/PRIORITY

The present invention claims priority to U.S. Provisional Patent Application No. 62/928,333 filed Oct. 30, 2019, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### STATEMENT REGARDING FEDERALLY SPONSORED RESEARCH OR DEVELOPMENT

This invention was made with government support under Grant No. P20 GM121307 awarded by the National Institutes of Health. The government has certain rights in the invention.

### BACKGROUND

Stroke kills about 140,000 Americans each year, or about 1 out of every 20 deaths. Someone in the United States has a stroke every 40 seconds. Every 4 minutes, someone dies of stroke. Every year, more than 795,000 people in the United States have a stroke. About 87% of all strokes are ischemic strokes, in which blood flow to the brain is blocked. Stroke costs the United States an estimated $34 billion each year. This total includes the cost of health care services, medicines to treat stroke, and missed days of work. Stroke is a leading cause of serious long-term disability. There is no current treatment that sufficiently protects against the damage of stroke, especially past the 4-minute window. Stem cells have been shown to be protective/reparative in some conditions, but their use can be complicated by sometimes fatal hazards associated with their intravenous injection, particularly in stroke models.

### SUMMARY

Wherefore, it is an object of the present invention to overcome the above-mentioned shortcomings and drawbacks associated with the current technology.

The present invention is related to cells and a method of creating enhanced human Placenta Mesenchymal Stem Cells (hPMSCs) comprising culturing the hPMSCs with cholesterol for one of between 24 and 96 hours and between 48 and 72 hours. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the stem cells are in a frozen state and stored in a cell number designed for stroke therapy administration. According to a further embodiment the stem cells are stored in units of one of 10 million, 20 million, 50 million, 100 million, 200 million, and 500 million. increasing ACE2 expression using diminazene, a described ACE2 activator

The present invention is related to exosomes and a method of creating enhanced exosomes from hPMSCs comprising culturing the hPMSCs with cholesterol for one of between 24 and 96 hours and between 48 and 72 hours, and then separating the enhanced exosomes from the hPMSCs. According to a further embodiment, media containing the hPMSCs and exosomes are centrifuged. According to a further embodiment a resulting supernatant is transferred to microcentrifuge tubes and re-centrifuged at an increased gravity to pellet extracellular vesicles (EVs) that include exosomes. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment exosomes are isolated from the EVs by one of two-step differential centrifugation step, use of Annexin V-coated magnetic beads, sucrose gradient centrifugation, immunoisolation, ExoMir® filtration technologies, ExoQuick® precipitation technologies, size filtration, and/or ultracentrifugation. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the exosomes are in a frozen state and stored in an amount designed for stroke therapy administration. According to a further embodiment the amount of exosomes is equivalent to an amount of exosomes that are separated from of one of 10 million, 20 million, 50 million, 100 million, 200 million, and 500 million hPMSCs.

The present invention is related to microparticles and a method of creating enhanced microparticles from hPMSCs comprising culturing the hPMSCs with cholesterol for one of between 24 and 96 hours and between 48 and 72 hours, and then separating the enhanced microparticles from the hPMSCs. According to a further embodiment, media containing the hPMSCs and microparticles are centrifuged. According to a further embodiment a resulting supernatant is transferred to microcentrifuge tubes and re-centrifuged at an increased gravity to pellet EVs that include microparticles. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment microparticles are isolated from the EVs by one of two-step differential centrifugation step, use of Annexin V-coated magnetic beads, sucrose gradient centrifugation, immunoisolation, ExoMir® filtration technologies, ExoQuick® precipitation technologies, size filtration, and/or ultracentrifugation. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the microparticles are in a frozen state and stored in an amount designed for stroke therapy administration. According to a further embodiment the amount of microparticles is equivalent to an amount of microparticles that are separated from of one of 10 million, 20 million, 50 million, 100 million, 200 million, and 500 million hPMSCs.

The present invention is related to EVs including exosomes and microparticles and a method of creating enhanced EVs from hPMSCs comprising culturing the hPMSCs with cholesterol for one of between 24 and 96 hours and between 48 and 72 hours, and then separating the enhanced EVs from the hPMSCs. According to a further embodiment, media containing the hPMSCs and EVs are centrifuged. According to a further embodiment a resulting supernatant is transferred to microcentrifuge tubes and re-centrifuged at an increased gravity to pellet EVs. According to a further embodiment, a second supernatant was aspirated, and EVs pellet was washed twice by centrifugation using 4° C. PBS/1 mM phenylmethylsulfonyl fluoride (PMSF) and pelleted a final time at 20,800 g for 15 min (4° C.), and supernatants were aspirated. According to a further embodiment the cholesterol is between a 1:100 dilution and 1:100 dilution, preferably between a 1:200 dilution and 1:500 dilution, and most preferably a 1:250 dilution. According to a further embodiment a lipid emulsion is further included in the hPMSCs culture. According to a further embodiment the lipid emulsion is a CD lipid concentrate at between a 1:25 and 1:500 dilution, preferably a 1:50 and 1:200 dilution, and most preferably a 1:100 dilution. According to a further embodiment the EVs are in a frozen state and stored in an amount designed for stroke therapy administration. According to a further embodiment the amount of EVs is equivalent to an amount of EVs that are separated from of one of 10 million, 20 million, 50 million, 100 million, 200 million, and 500 million hPMSCs.

The present invention further relates to methods and therapeutics comprising a plurality of hPMSCs, wherein the hPMSCs were cultured with cholesterol, such that the hPMSCs have an increased number of extracellular vesicles. According to a further embodiment the hPMSCs have one of 2, 3, 4, 5, and 6 times the number of extracellular vesicles as a hPMSC that is cultured in the absence of cholesterol.

The present invention is related to therapeutics and methods of treating a stroke in a patient comprising delivering stem cells into a peritoneum of the patent. According to a further embodiment the stem cells are MSC. According to a further embodiment the stem cells are hPMSC. According to a further embodiment the stroke is one of stroke is one of occlusive, post-occlusive, hemorrhagic, and transient ischemic injury. According to a further embodiment the method further comprises the step of delivering a clot busting compound into a blood stream of the patient. According to a further embodiment the stem cells are delivered into the peritoneum of the patent one of before, coincident with, and after the clot busting compound is introduced into the patient, and wherein the clot busting compound is tissue plasminogen activator. According to a further embodiment the stem cells are delivered via injection. According to a further embodiment a number of stem cells introduced is between one of 1 million and 5 billion, 10 million and 2 billion, and 100 million and 500 million. According to a further embodiment the stem cells are immortalized. According to a further embodiment the stem cells are lentivirally immortalized. According to a further embodiment the stem cells are delivered one of before the stroke occurs, coincident with the stroke occurring, within 1 hour of the stroke occurring, and between an earliest of 0.5, 1, 4, 12, and 24 hours of the stroke occurring and a latest of 1, 4, 12, and 24 hours of the stroke occurring. According to a further embodiment the stem cells have an increased number of extracellular vesicles. According to a further embodiment the stem cells have been cultured with a sterol. According to a further embodiment the sterol is cholesterol. According to a further embodiment the stem cells are cultured with cholesterol for one of between 24 and 96 hours and between 48 and 72 hours. According to a further embodiment a lipid emulsion is further included in the stem cells are additionally culture. According to a further embodiment the stem cells are in a frozen state and stored in a cell number designed for stroke therapy administration. According to a further embodiment the stem cells are stored in units of one of 10 million, 20 million, 50 million, 100 million, 200 million, and 500 million.

The present invention further relates to methods and therapeutics comprising a plurality of hPMSCs, wherein the hPMSCs were cultured with cholesterol, such that the hPMSCs have an increased number of extracellular vesicles. According to a further embodiment the hPMSCs have one of 2, 3, 4, 5, and 6 times the number of extracellular vesicles as a hPMSC that is cultured in the absence of cholesterol.

The present invention further relates to therapeutics and methods of treating a stroke in a patient comprising administering a therapy to cause reperfusion in the patient, injecting immortalized hPMSC into a peritoneum of the patent substantially at a same time as the reperfusion therapy is administered wherein the stroke is one of stroke is one of occlusive, post-occlusive, hemorrhagic, and transient ischemic injury, a number of stem cells introduced is between one of 1 million and 5 billion, 10 million and 2 billion, and 100 million and 500 million, the stem cells have been cultured with cholesterol and a lipid emulsion for between 24 and 96 hours.

The inventors describe the use of intraperitoneal stem cells as a safe alternative which provides nearly complete protection against acute stroke injury in the occlusive model of stroke.

The present invention relates to the use of immortalized placental stem cells (IPSC) and/or extracellular vesicles derived therefrom to enhance therapeutic recovery from tissue damage and ischemia-reperfusion injury and delayed organ function.

The present invention relates to a novel, more effective and safer approach to use human placenta mesenchymal stem cells as protective agents in stroke models and in clinical stroke as well as ischemia reperfusion injury. A further embodiment of the invention is to use frozen stem cells which are thawed on demand to provide a more easily accessible option for patients undergoing stroke therapy. This treatment is intended to provide cell therapy beyond the 4 hour window now used for stroke. This protection is mediated at least in part by stem cell-derived exosome/microparticles and their expression of angiotensin converting enzyme 2. The numbers and efficacy of these particles may be increased by supplementing stem cells with cholesterol supplements prior to stem cell or exosome administration and by increasing ACE2 expression using dim inazene, a described ACE2 activator.

One embodiment of the present invention uses lentivirally immortalized human placenta derived, autologous or other origin stem cell as a therapeutic approach to maintain blood flow and preserve tissue integrity. This invention involves the creation of sufficient cells and/or extracellular vesicles which can be frozen for later therapeutic administration. Additionally, these cells enable the creation of much greater recovery of extracellular vesicles to permit administered intravenous as well as intraperitoneal administration. Importantly, the process makes it possible to obtain large enough numbers of cells for a clinical ‘dose’ which can provide protection against stroke injury (tested in the middle cerebral artery occlusion model of stroke (MCAO)). We found that administration of 500,000 stem cells into the peritoneum was significantly protective of tissue structure and neurological outcomes in stroke models. However, scaling this to human therapy could require 1-2 billion cells which could be difficult to obtain based on typical cell proliferation rates. However, these IPSC grow extremely rapidly, provide equivalent protection as non-transformed cells and represent a novel therapeutic. Similarly, vesicles derived from these cells are also effective and therapeutic and may have improved activity towards maintenance/restoration of blood flow and tissue function.

According to one embodiment, the disclosed approach would be used to treat individuals who are seen at emergency departments for thrombotic strokes, particularly when tissue plasminogen activator (tPA) is administered. This treatment when given after a stroke would prevent the progressive brain tissue destruction, loss of function and behavioral and motor disturbances which often accompany stroke even when tPA is administered.

According to one embodiment, a cell-based exosome ‘product’ would be provided which would ultimately be used after every tissue plasminogen treatment for stroke ‘clot-busting’. This product could be banked and shipped and used at later dates and allow for much greater quality control. It is much safer than the intravenous use of stem cells.

According to one embodiment, the present invention relates to ischemic stroke injury and therapy. A disclosed method overcomes the relatively dangerous current use of stem cells in an occlusive crisis in the brain vasculature. Because ‘clot busting’ anticipates the removal of a blood clot using tissue plasminogen activator (TPA), intravenous stem cells present potential risks. By comparison intraperitoneal stem cells (administered into the abdomen) are much safer and highly effective, potentially almost completely eliminating stroke injury in the middle cerebral artery occlusion model.

One embodiment of the present invention involves administering human placenta stem cells by an intraperitoneal route prior to or following stroke onset and/or prior to, concurrent with, or following the administration of “clot busting” therapies.

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore, the claimed invention should not necessarily be construed as limited to addressing any of the particular problems or deficiencies discussed herein.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which are incorporated in and constitute a part of the specification, illustrate various embodiments of the invention and together with the general description of the invention given above and the detailed description of the drawings given below, serve to explain the principles of the invention. It is to be appreciated that the accompanying drawings are not necessarily to scale since the emphasis is instead placed on illustrating the principles of the invention. The invention will now be described, by way of example, with reference to the accompanying drawings in which:

FIGS. 1A-1D are brain slice photographs, graphs, Laser Speckle Imaging, and cell micrographs of sham group, MCAO mice, and MCAO mice with intraperitoneal injected stem cells following reperfusion. FIG. 1A shows the area of infarction (‘white’ area) in each brain slice was detected using 2,3,5-triphenyltetrazolium chloride (TTC) staining and measured using the ImageJ program (NIH). Unstained (‘white’) regions in brain slices were combined from each brain to generate a composite tissue injury score for each subject. Each point represents one animal subject (n=7 sham, n=6 MCAO, n=8 MCAO+hPMSC). Significant differences were determined by one-way analysis of variance (ANOVA) and Bonferroni post hoc tests. Comparison of MCAO with sham and hPMSCtreated MCAO groups, respectively were significantly different (p=0.0001 and p<0.0001). There were no significant differences in infarct size between hPMSC-treated MCAO and sham groups (p=0.32). FIG. 1B shows total cerebral perfusion measured using Laser Speckle imaging. ‘Perfusion’ reflects total cerebral flow signal measured in selected tissue areas. Measurements are expressed as perfusion units (PU), using a fixed scale (arbitrary units). Significant differences were observed between cerebral perfusion in MCAO (n=5) and sham (n=4) mice (p=0.0004) but not between MCAO+hPMSC and sham groups (p=0.03, n=4) using one-way ANOVA and Bonferroni post hoc tests. hPMSC-treated MCAO mice showed significant (P=0.01) preservation of perfusion compared to non-treated post-MCAO group (two-tailed Student's t-test). FIGS. 1C and 1D show cerebral perfusion in each pair of ipsilateral (FIG. 1C) and contralateral (FIG. 1D) brain hemispheres were normalized to the total perfusion of the same mouse. (FIG. 1C) A large (68%) decrease of cerebral perfusion into the ipsilateral side of MCAO brains was observed compared to the sham operated group (one-way ANOVA and Bonferroni post hoc tests, p=0.002); (FIG. 1D) A significant increase in cerebral perfusion into the contralateral hemisphere of MCAO brains was observed compared to the sham group (one-way ANOVA and Bonferroni post hoc tests, p=0.001). hPMSC treatment significantly preserve the normal distribution of blood flow between the hemispheres (far right bars; FIGS. 1C and 1D); significant differences determined by Student's t-test, P=0.01 and P=0.01 for comparison of hPMSC-treated MCAO mice to untreated MCAO ipsilateral (FIG. 1C)/contralateral (FIG. 1D) hemispheres, respectively. FIG. 1E shows neurological scores 24 hours after reperfusion. Significant differences in neurological scores were seen between untreated MCAO and hPMSC-treated MCAO to sham group (one-way ANOVA and Bonferroni post hoc tests, p<0.0001). There was a significant improvement in neurological score in hPMSC-treated MCAO mice compared to untreated MCAO (P<0.0001, two-tailed Student's t-test analysis). FIG. 1F shows neuronal degeneration in MCAO w/hPMSC therapy. Neurons (black arrows), degenerating neurons (black arrows head), glial cells (black asterisk), and spongiform regions (white arrows) were marked using Nissl histological staining of brain sections in untreated and hPMSC-treated MCAO groups. FIG. 1G-1I shows two-tailed Student's t-test analysis revealed significant differences in proportions of (FIG. 1G) neurons (P=0.02), (FIG. 1H) degenerating neurons (P=0.04), and (FIG. 1I) glial cell (P=0.02) numbers in hPMSC-treated MCAO brain sections compared to untreated MCAO sections. In all graphs, data represent means±SEM.

FIGS. 2A-2O show hPMSCs maintain the blood brain barrier integrity against MCAO in mice. FIG. 2A shows BBB disruption assessed by quantitation of Evans blue (EB) leakage into the brain of sham, MCAO, hPMSC-treated MCAO mice 24 hours following reperfusion. Significant differences in EB leakage were observed between MCAO versus sham and hPMSC-treated MCAO mice using one-way ANOVA and Bonferroni post hoc tests, P=0.0001 and Student t-test analysis, P=0.004, respectively. One-way ANOVA and Bonferroni post hoc tests analysis indicated no significant differences between hPMSC-treated MCAO versus sham mice. FIG. 2B shows a schematic illustration of in vitro experimental model of ischemic condition. FIG. 2C shows barrier function of human brain endothelial cell (hCMEC-D3) monolayers under normoxia and oxygen glucose deprivation reperfusion (OGDR) condition measured using biotinylated gelatin FITC-avidin permeability assay. One-way ANOVA and Bonferroni post hoc tests were used to assess significant differences in fluorescence intensity of D3 monolayers under nomoxia versus OGDR (P=0.0001) and hPMSC-cocultured D3 monolayers OGDR (P=0.0003). Significant differences in fluorescence intensity were observed for D3 monolayers under OGDR versus hPMSC-co-cultured under OGDR by Student t-test analysis, P=0.02. (I to III) representative images of biotinylated gelatin FITC-avidin permeability assay of D3 monolayers under (I) normoxia, (II) OGDR, and (III) OGDR+hPMSC conditions. Scale bars, 100 μm (I to III). FIG. 2D shows Western blots for protein expression of ZO-1, Claudin-1, Occludin, and b-tubulin in D3 monolayers under normoxia and OGDR (with or without hPMSC). Quantification of ZO-1 (FIG. 2E), claudin1 (FIG. 2F), and occludin (FIG. 2G) protein expression normalized to b-tubulin protein expression. No significant differences observed in the protein expression of ZO-1 (E; P=0.8), claudin1 (F; P=0.76), and occludin (G; P=0.64) of D3 monolayers under OGDR condition versus OGDR+hPMSC group using Student t-test analysis. All graph data show the means±SEM. Western blots and quantification of VE-cadherin (FIGS. 2H and 2I) and a-catenin (FIGS. 2L and 2M) protein expression normalized to b-tubulin. Significant differences in (FIGS. 2I and 2M) were determined by Student t-test analysis for comparisons between OGDR and OGDR+hPMSC, P=0.01 and P=0.006, respectively. Immunofluorescence staining and quantification of VE-cadherin (FIGS. 2J and 2K) and a-catenin (FIGS. 2N and 2O). Spatial localization of both VE-cadherin (FIG. 2K) and a-catenin (FIG. 2O) of D3 monolayers improved with hPMSC treatment under OGDR condition. Fluorescence intensity of VE-cadherin and a-catenin (green color; FIG. 2K and FIG. 2O, respectively) was normalized to the cell numbers (blue color, DAPI-stained nuclear). Significant differences of fluorescence intensity of VE-cadherin (FIG. 2J; P=0.01) and a-catenin (FIG. 2N; P=0.01) were observed in hPMSC-treated D3 monolayers under OGDR condition using Student t-test analysis. Scale bars, 100 μm (FIGS. 2K and 2O).

FIGS. 3A-3F show hPMSC-released EVs contribute to SC-based protection in the MCAO model. In FIG. 3A, flow cytometry was used to evaluate numbers of extracellular vesicles (EVs) released from hPMSC treated with or without (10 mM) methyl beta cyclodextrin (MβCD) for 48 h. Student t-test analysis revealed significant differences in the number of EV released from hPMSC compared to MβCD-treated hPMSC (****p=0.0008). Shown in FIG. 3B, the infarcted area was assessed by TTC staining. Significant differences of infarction were measured between MCAO mice versus MCAO+hPMSC (****p<0.0001) and MCAO+MbCD-treated hPMSC (NS, p=0.93) using one-way ANOVA. Significant increase of infarcted area was observed in MCAO+MbCD-treated hPMSC versus MCAO+hPMSC mice (****p<0.0001, Student t-test). Shown in FIG. 3C, Neurological scores of MβCD-treated hPMSC mice were comparable to MCAO group (NS, p=0.54, one-way ANOVA). Significant differences of neurological scores were observed in MCAO+hPMSC mice versus MCAO group (****p<0.0001; one-way ANOVA) and MβCD-treated hPMSC mice (****p=0.0001; Student t-test). FIG. 3D shows significant differences of the perfusion into the brain of MCAO+hPMSC mice to MCAO (*p=0.03, one-way ANOVA) and MβCD-treated hPMSC (***p=0.009, Student t-test) group were measured. No significant differences were detected in MCAO group compared to MβCD-treated hPMSC group (NS, p=0.82, one-way ANOVA). FIG. 3E shows significant decrease of blood flow into the ipsilateral hemisphere of MCAO (*p=0.02, one-way ANOVA) and MCAO+MβCD-treated hPMSC (*p=0.02, Student t-test) compared to hPMSC-treated MCAO group. Changes in the ipsilateral perfusion of MCAO and MCAO+MβCD-treated hPMSC groups were not significant (NS, p=0.99, one-way ANOVA). FIG. 3F shows significant increase of blood flow into the contralateral hemisphere of MCAO (***p=0.008, one-way ANOVA) and MCAO+MβCD-treated hPMSC (**p=0.01, Student t-test) brains was detected compared to MCAO+hPMSC groups. No significant differences of contralateral perfusion were observed between MCAO and MCAO+MβCD-treated hPMSC mice (NS, p=0.85, one-way ANOVA). All graph data show the means±SEM.

FIGS. 4A-4E show cholesterol/lipid supplementation enhances the protective capacity of hPMSCs in the MCAO model. FIG. 4A shows cholesterol contents of hPMSC were evaluated using Oil Red O staining which stains lipid droplets and intracellular cholesterol. No significant changes were observed in staining of intracellular cholesterol (NS, p=0.7, Student t-test). FIG. 4B shows the number of EVs released from hPMSC and cholesterol-treated hPMSC was calculated using flow cytometric analysis. Significant increase in the number of EVs released from cholesterol-treated hPMSC was detected compared to untreated hPMSC (**p=0.01, Student t-test). FIGS. 4C-4E show the protective potential of 1×10⁵ in 100 μl HBSS of hPMSC and cholesterol-treated hPMSC was compared in the MCAO model. Significant differences of (FIG. 4C) infarcted area (TTC staining; NS, p=0.9 and *p=0.02), (FIG. 4D) total cerebral blood flow (NS, p=0.7 and **p=0.003), and (FIG. 4E) neurological scores (NS, p=0.67 and **p=0.005) were measured using one-way ANOVA for comparison of MCAO mice with MCAO+hPMSC and MCAO+Chl-hPMSC groups. FIG. 4E shows ipsilateral perfusion improved in MCAO+Chl-hPMSC mice compared to MCAO (*p=0.04, one-way ANOVA) and MCAO+hPMSC (**p=0.002, Student t-test) groups. Significant decrease of blood perfusion into ipsilateral hemisphere of both MCAO (***p=0.001) and MCAO+hPMSC (***p=0.0002) groups versus sham mice was observed using one-way ANOVA. FIG. 4F shows significant increase in contralateral hemisphere of MCAO (**p=0.009) and MCAO+hPMSC (***p=0.0003) groups was detected compared to sham mice. Redistribution of contralateral perfusion in MCAO+Chl-hPMSC mice was not significant compared to MCAO (NS, p=0.07, one-way ANOVA) groups. No significant differences were detected in ipsilateral (FIG. 4E; p=0.4) and contralateral (FIG. 4F; p=0.58) hemispheres of MCAO mice compared to MCAO+hPMSC mice by one-way ANOVA. All graph data show the means±SEM.

FIGS. 5A-5F show that IV injection of EVs derived from Chl-treated hPMSCs is protective in the MCAO model. Shown in FIG. 5A, net reduction in the expression of phosphatidylserine (PS) on the surface of EVs was revealed by significant decrease in annexin V positive EVs using flow cytometric analysis (***p=0.0002, Student t-test). Shown in FIGS. 5B-5E, the protective potential of IV injection of EVs derived from Chl-treated hPMSC (2×10⁶ EVs in 100 μl HBSS) was evaluated in the MCAO model. Significant differences in infarcted area (FIG. 5B; **p=0.01), total cerebral perfusion (FIG. 5C; **p=0.01), ipsilateral perfusion (FIG. 5D; *p=0.05), and neurological scores (FIG. 5F; **p=0.01) of MCAO+Chl-treated EVs and untreated MCAO groups were identified using Student t-test analysis. All the above parameters: FIG. 5B; infarcted area (p=0.07), FIG. 5C; total perfusion (p=0.98), FIG. 5D; ipsilateral perfusion (p=0.08), FIG. 5E; contralateral perfusion (p=0.08), FIG. 5F; neurological scores (p=0.08)) in MCAO+Chl-treated EVs mice were comparable to the sham group (one-way ANOVA). All graph data show the means±SEM.

FIGS. 6A-6D are brain slice photographs, graphs and Laser Speckle Imaging of MCAO mice versus sham group following reperfusion. FIG. 6A shows significant differences of infarcted area (TTC staining) in MCAO mice (n=10) versus sham group (n=7) was detected 24 hours after reperfusion using Student t-test analysis, P<0.0001. FIG. 6B shows there were no significant differences between MCAO (n=4) and sham group (n=4) at time point of 4 hours following reperfusion (P=0.32, Student t-test analysis). FIG. 6C shows significant differences of total perfusion into the brain (Laser Speckle Imaging) of MCAO mice (n=4) versus sham group (n=4) was detected 24 hours after reperfusion using Student t-test analysis, P=0.0008. FIG. 6D significant differences of neurological scores in MCAO mice compared to sham group was detected 24 hours after reperfusion using Student t-test analysis, P<0.0001.

FIGS. 7A-7G show characteristics of hPMSC. FIG. 7A shows that calcein AM staining showed spindle shape of hPMSCs in culture. Scale bar, 100 μm. Fluorescence-activated cell sorting (FACS) analysis detected the expression of CD73 (FIG. 7B) and CD90 (FIG. 7C) on hPMSC. Immunostaining of hPMSC was positive for markers of CD44 (FIG. 7D) and Oct3/4 (FIG. 7E). DAPI used for nuclear staining (FIGS. 7A, 7D and 7E). FIGS. 7F and 7G demonstrate that FACS analysis showed negative expression of HLA-DR and CD34 markers on hPMSC.

FIG. 8A-8F show protein expression of tight junction proteins (ZO-1, claudin-1, occludin) and adherens junction proteins (VE-cadherin and a-catenin) of hCMEC-D3 monolayers under oxygen glucose deprivation reperfusion (OGDR) condition at different time points of 6, 12, and 16 hours that was measured by western blot analysis. Statistical differences were determined by two-way ANOVA and Sidak's multiple comparisons tests for comparisons to OGDR condition at 6, 12, and 12 time points. For quantification, expression of each protein normalized to protein expression of b-tubulin. Significant differences in protein expression of each protein under OGDR compared to normoxia at different time points as follow: (FIG. 8A) ZO-1 (6 h; P=0.99, 12 h; P=0.03, 16 h; P=0.05); (FIG. 8B) claudin-1 (6 h; P=0.11, 12 h; P=0.003, 16 h; P=0.02); (FIG. 8C) occludin (6 h; P=0.14, 12 h; P=0.002, 16 h; P=0.0.005); (FIG. 8D) VE-cadherin (6 h; P=0.50, 12 h; P=0.04, 16 h; P=0.05); (FIG. 8E) a-catenin (6 h; P=0.96, 12 h; P=0.02, 16 h; P=0.02). FIG. 8F shows representative western blots.

FIGS. 9A-9F show expression of Tight/adherens junctional proteins of hCMEC-D3 monolayers co-cultured with hPMSCs under normoxia. Protein expression of tight junctional proteins (ZO-1, claudin-1, occludin) and adherens junctional proteins (VE-cadherin and a-catenin) of hCMEC-D3 monolayers under normal (normoxia) condition when co-cultured (contact independently) with hPMSC for 24 hours and 48 hours compared to the correspondent control using western blot analysis. Statistical differences were measured by two-way ANOVA and Sidak's multiple comparisons tests. For quantification, expression of each protein normalized to protein expression of β-tubulin. No significant differences were detected in protein expression of each protein in hCMEC-D3 monolayers±co-cultured hPMSC (24, 48 hours) as shown here: (FIG. 9A) ZO-1 (24 h; p=0.96, 48 h; p=0.64); (FIG. 9B) claudin-1 (24 h; p=0.98, 48 h; p=0.78); (FIG. 9C) occludin (24 h; p=0.67, 48 h; p=0.78); (FIG. 9D) VE-cadherin (24 h; p=0.46, 48 h; p=0.69); (FIG. 9E) a-catenin (24 h; p=0.92, 48 h; p=0.28). (FIG. 9F) Representative western blots.

FIGS. 10A-10F show Expression of Tight/adherens junctional proteins of hCMEC-D3 monolayers co-cultured with hCMEC-D3 under normoxia. Protein expression of tight junctional proteins (ZO-1, claudin-1, occludin) and adherens junctional proteins (VE-cadherin and a-catenin) of hCMEC-D3 monolayers under normal (normoxia) condition when co-cultured (contact independently) with hCMEC-D3 for 24 hours and 48 hours compared to the correspondent control using western blot analysis. Statistical differences were measured by two-way ANOVA and Sidak's multiple comparisons tests. For quantification, expression of each protein normalized to protein expression of a-tubulin. Significant differences in protein expression of each protein in hCMEC-D3 monolayers±co-cultured hCMEC-D3 (24, 48 hours) were measured as follow: (FIG. 10A) ZO-1 (24 h; p=0.99, 48 h; p=0.74); (FIG. 10B) claudin-1 (24 h; p=0.97, 48 h; p=0.92); (FIG. 10C) occludin (24 h; p=0.89, 48 h; p=0.88); (FIG. 10D) VE-cadherin (24 h; p=0.73, 48 h; p=0.81); (FIG. 10E) a-catenin (24 h; p=0.09, 48 h; p=0.99). (FIG. 10F) Representative western blots.

FIGS. 11A-11C are micrographs of stained hPMSCs and a line graph. Shown in FIG. 11A, to track intraperitoneally injected hPMSCs in the MCAO model, hPMSCs were labelled using CytoID red long-term cell tracer kit. Shown in FIG. 11B, blood was collected at time points of 2, 6, and 24 hours after injection, and red-labelled cells were counted by a Nikon video imaging system Eclipse E600FN; using a 20× objective lens. Scale bars, 100 μm. Shown in FIG. 11C, IF staining of hPMSC with anti-human nuclear (Hu-Nu; green) antibody and mouse vascular endothelial cells with anti-mouse CD31 antibody (red); DAPI was used to stain the nuclear of the cells. There was no hPMSC localized in the brain (no green signal was detected). Scale bars, 100 μm.

FIGS. 12A and 12B show IP injection of hPMSC significantly improve neurological function and protect injury in MCAO mice after 24 hours reperfusion. FIG. 12A is a graph showing neurological score for Sham, MCAO, and MCAO+SC. FIG. 12B is a set of brain slice photographs of Sham, MCAO, and MCAO, and a graph presenting % infarcted area for each of Sham, MCAO, and MCAO+SC brain slices.

FIGS. 13A-13C show total blood flow was restored in MCAO brain after 24 hours perfusion with hPMSC administration. FIG. 13A shows total cerebral perfusion measured using Laser Speckle imaging for Sham, MCAO, and MCAO+SC brains, and a graph presenting the perfusion per square mm for each of Sham, MCAO, and MCAO+SC. FIG. 13B shows contralateral hemisphere perfusion normalized to total blood flow for each of Sham, Sham, MCAO, and MCAO+SC. FIG. 13C shows ipsilateral hemisphere perfusion normalized to total blood flow for each of Sham, Sham, MCAO, and MCAO+SC.

FIGS. 14A-14C show that EVs contribute to hPMSC based protection in the MCAO model. FIG. 14A is four brain slice photographs of Sham, MCAO, MCAO+SC, and MCAO+MBCD+SC, and a graph presenting % infarcted area for each of Sham, MCAO, MCAO+SC, and MCAO+MBCD+SC brain slices. FIG. 14B is a graph showing neurological score of each of Sham, MCAO, MCAO+SC, and MCAO+MBCD+SC. FIG. 14C shows total cerebral perfusion measured using Laser Speckle imaging for Sham, MCAO, MCAO+SC, and MCAO+MBCD+SC and a graph presenting the perfusion per square mm for each of Sham, MCAO, MCAO+SC, and MCAO+MBCD+SC.

FIGS. 15A—are 15D show cholesterol treatment of hPMSC. FIG. 15A shows a schematic representation of the cholesterol enhancement treatment, and steps of testing, and two photomicrographs cholesterol contents of hPMSC evaluated using Oil Red O staining. FIG. 15B is a graph showing optical density at 492 nm. FIG. 15C shows a graph of the number of EVs released from non-cholesterol treated (‘unmodified”) hPMSC calculated using flow cytometric analysis, and a graph of total EVs released for unmodified SC and enhanced SC. FIG. 15D shows a graph of the number of EVs released from cholesterol-treated (or “enhanced”) hPMSC and a graph of Anx V+ for each of unmodified SC and enhanced SC.

FIGS. 16A-16C the results of IP injection of both 1×10\hat{ }5 unmodified hPMSCs and 1×10\hat{ }5 hPMSCs treated with cholesterol for 72 hours (enhanced hPMSCs). FIG. 16A shows four brain slice photographs of Sham, MCAO, MCAO+SC (SC), and MCAO+Enhanced SC (Chl-SC), and a graph presenting % infarcted area for each of Sham, MCAO, SC, and Chl-SC brain slices. FIG. 16B is a graph showing behavioral function through neurological score of each of Sham, MCAO, SC, and Chl-SC. FIG. 16C shows total cerebral perfusion measured using Laser Speckle imaging for Sham, MCAO, SC, and Chl-SC and a graph presenting the perfusion per square mm for each of Sham, MCAO, SC, and Chl-SC.

FIGS. 17A-17C show the results of IV injection of EVs derived from cholesterol treated (enhanced) hPMSCs. FIG. 17A shows three brain slice photographs of Sham, MCAO, and EVs from enhanced SC (Chl-EV), and a graph presenting % infarcted area for each of Sham, MCAO, and Chl-EV brain slices. FIG. 17B is a graph showing behavioral function through neurological score of each of Sham, MCAO, and Chl-EV. FIG. 17C shows total cerebral perfusion measured using Laser Speckle imaging for Sham, MCAO, and Chl-EV and a graph presenting the perfusion per square mm for each of Sham, MCAO, and Chl-EV.

FIGS. 18A-18C show the results of IP injection of hPMSCs frozen in cholesterol supplemented freezing media. FIG. 18A shows three brain slice photographs of Sham, MCAO, and frozen SC (F-SC), and a graph presenting % infarcted area for each of Sham, MCAO, and F-SC brain slices. FIG. 17B is a graph showing behavioral function through neurological score of each of Sham, MCAO, and F-SC. FIG. 18C shows total cerebral perfusion measured using Laser Speckle imaging for Sham, MCAO, and F-SC and a graph presenting the perfusion per square mm for each of Sham, MCAO, and F-SC.

### DETAILED DESCRIPTION

The present invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40% means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

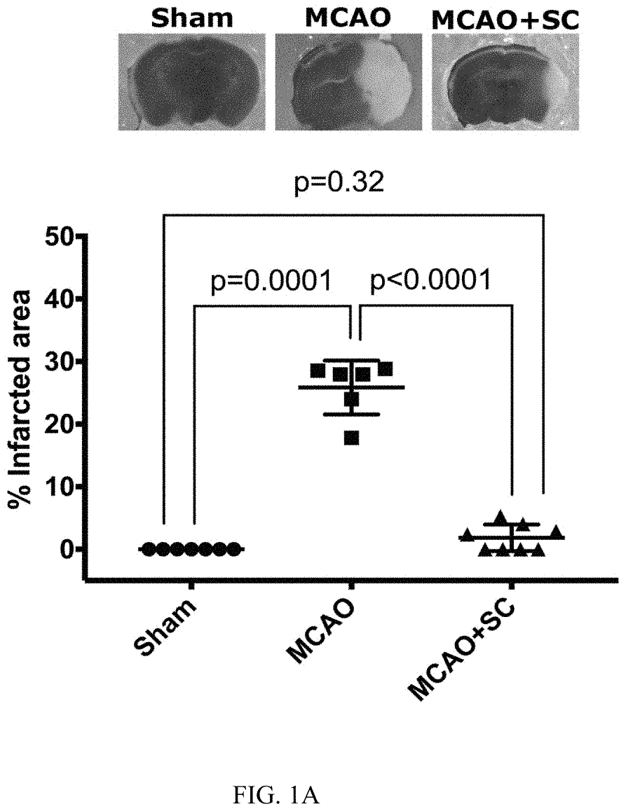

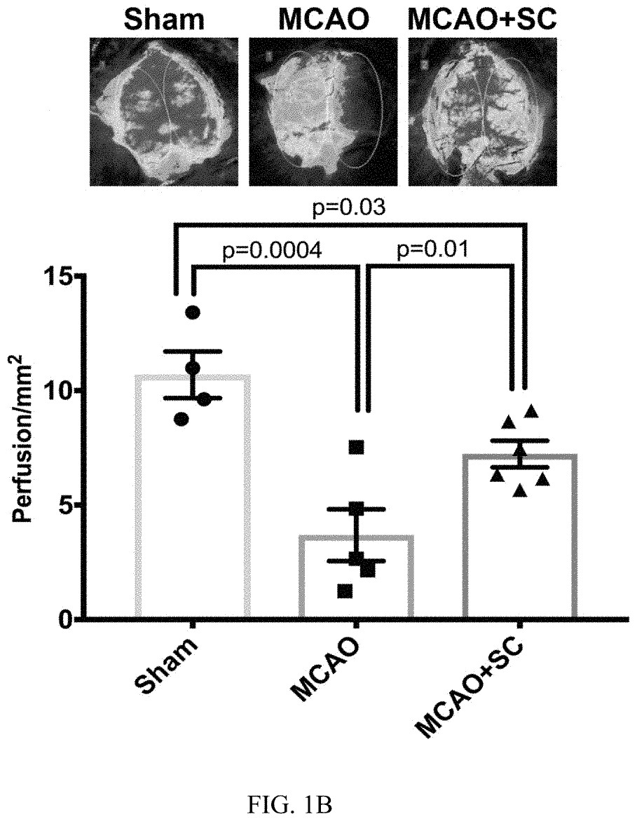

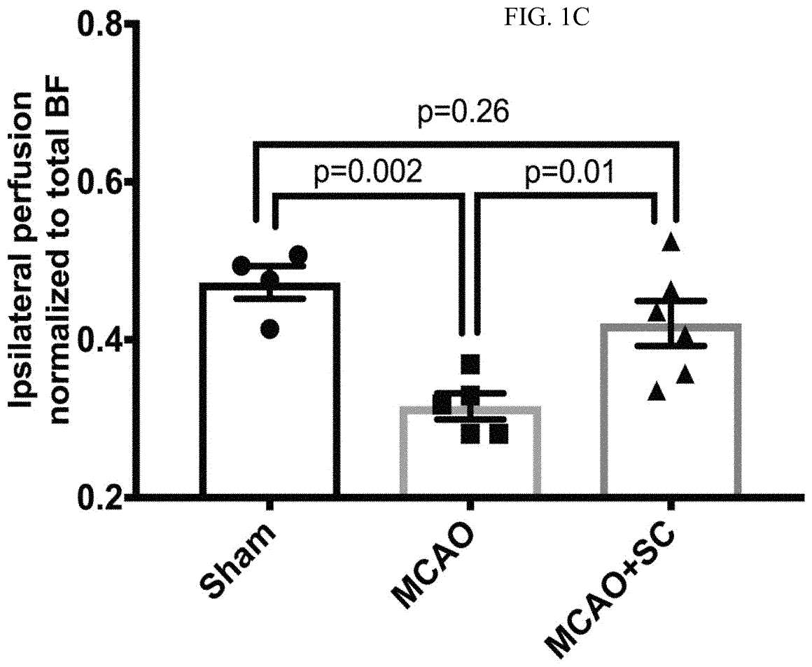

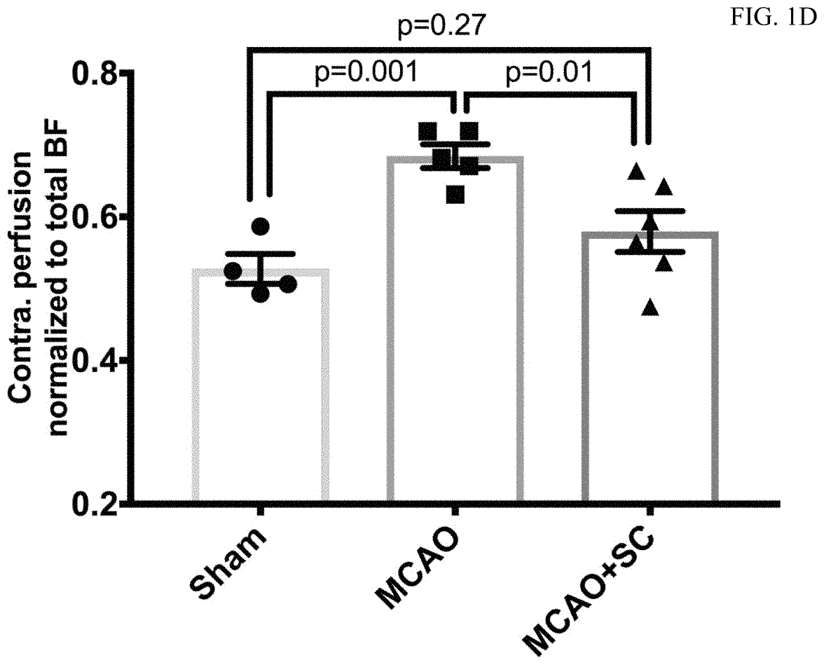

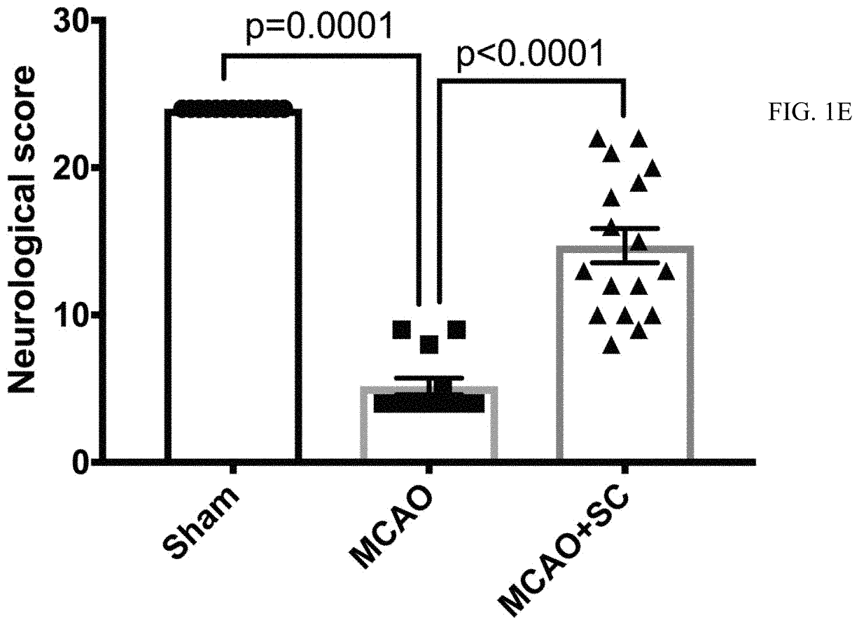

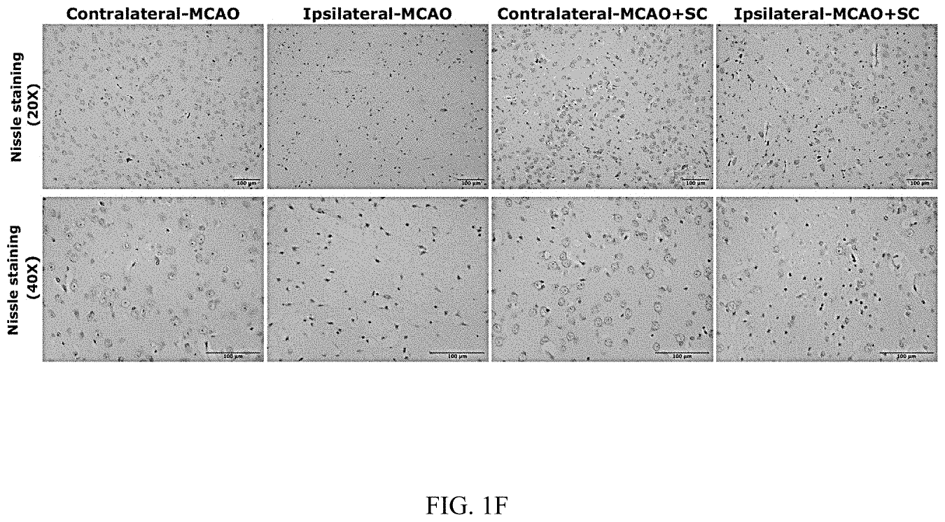

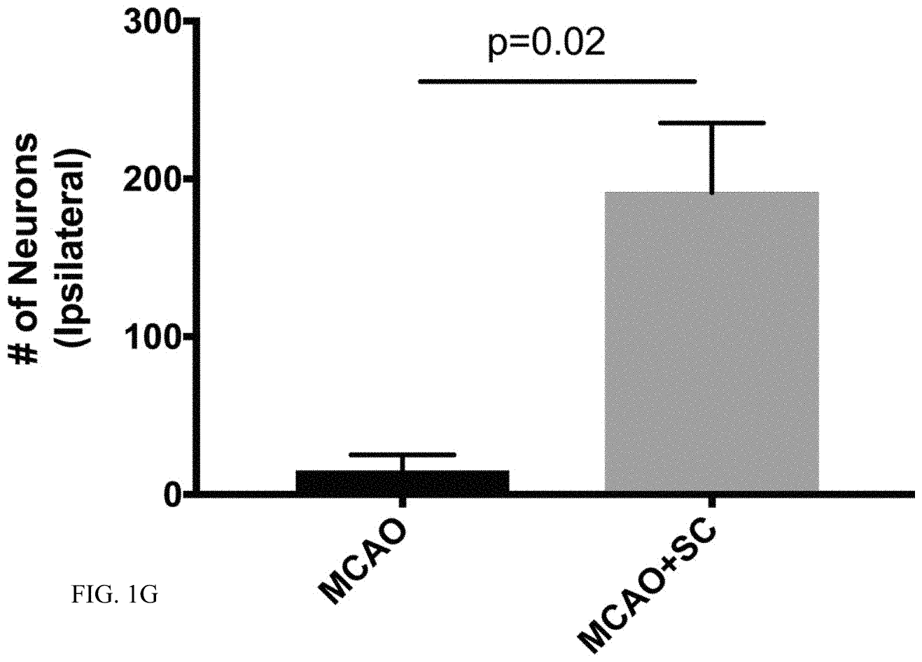

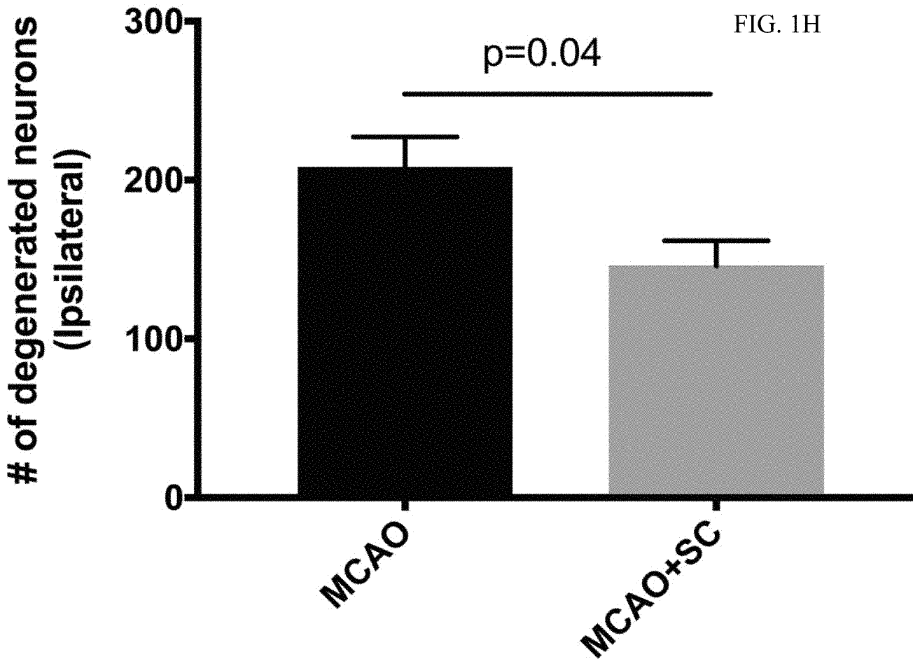

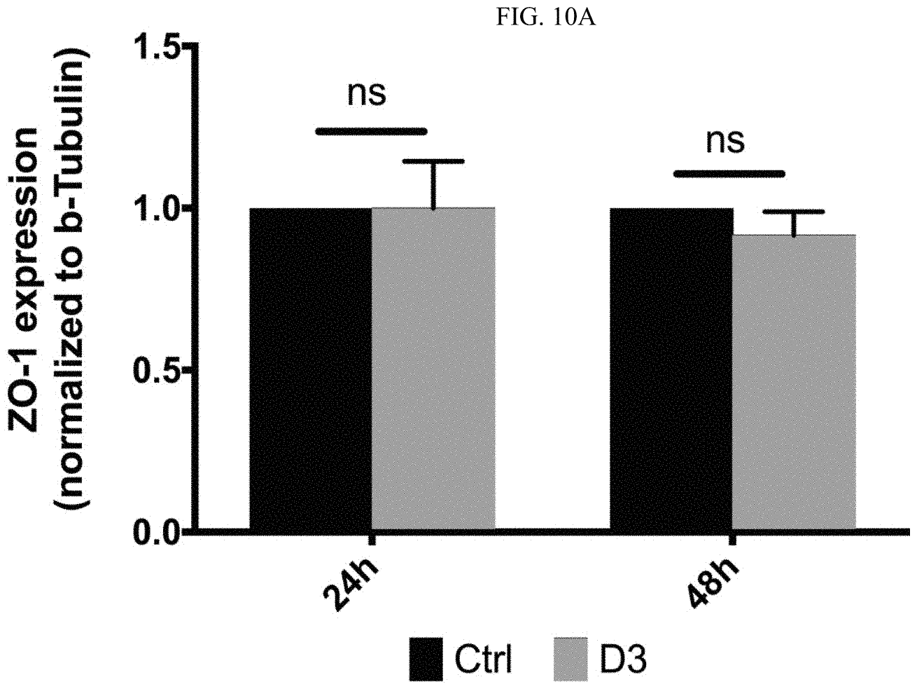

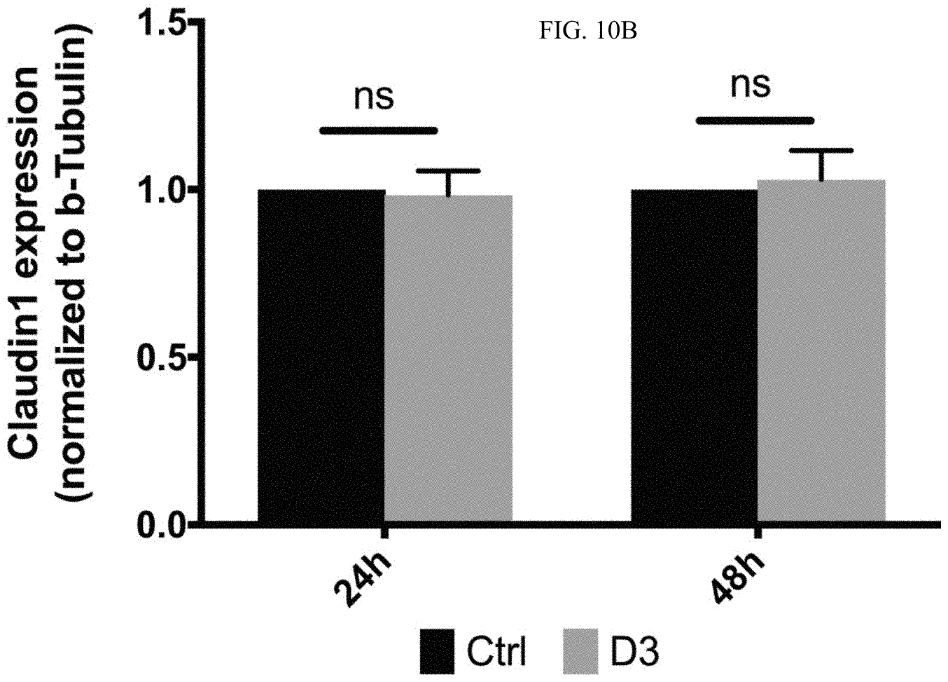

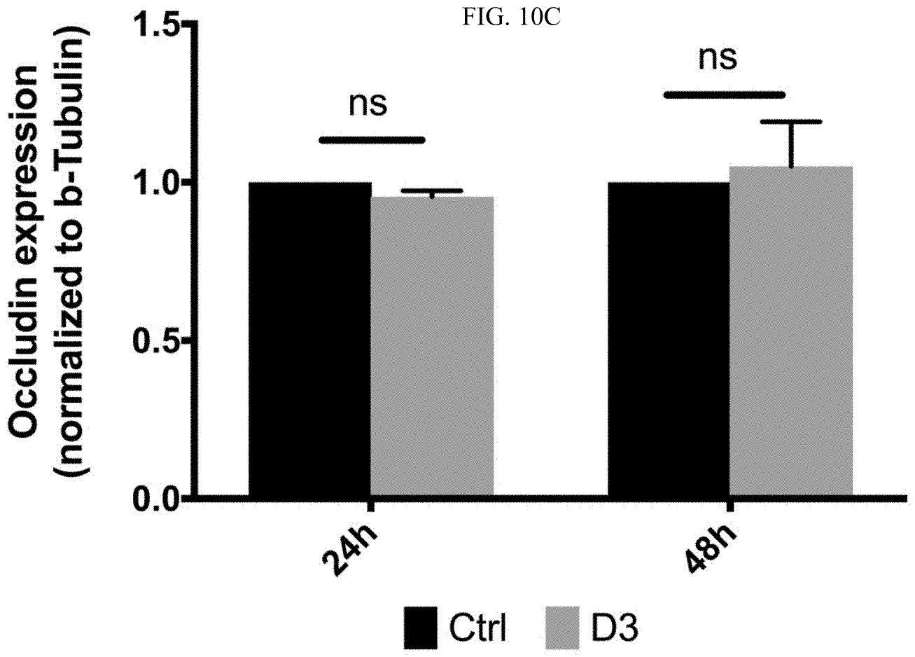

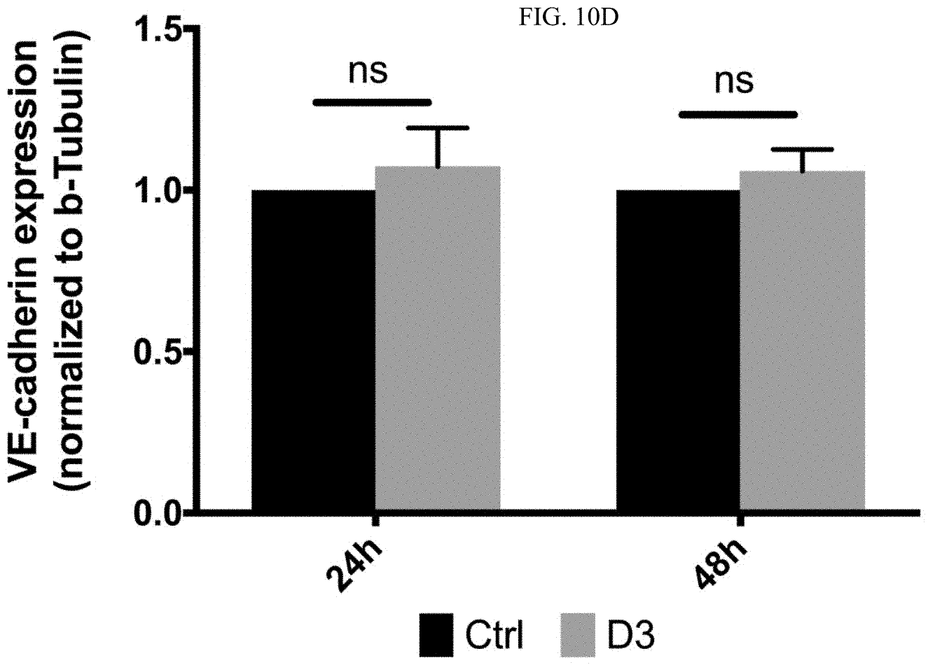

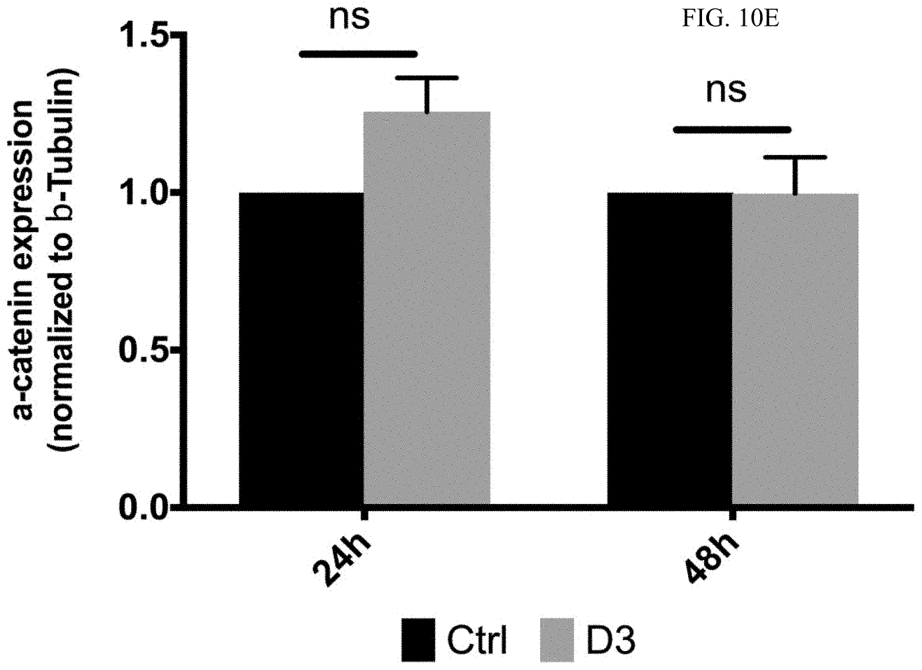

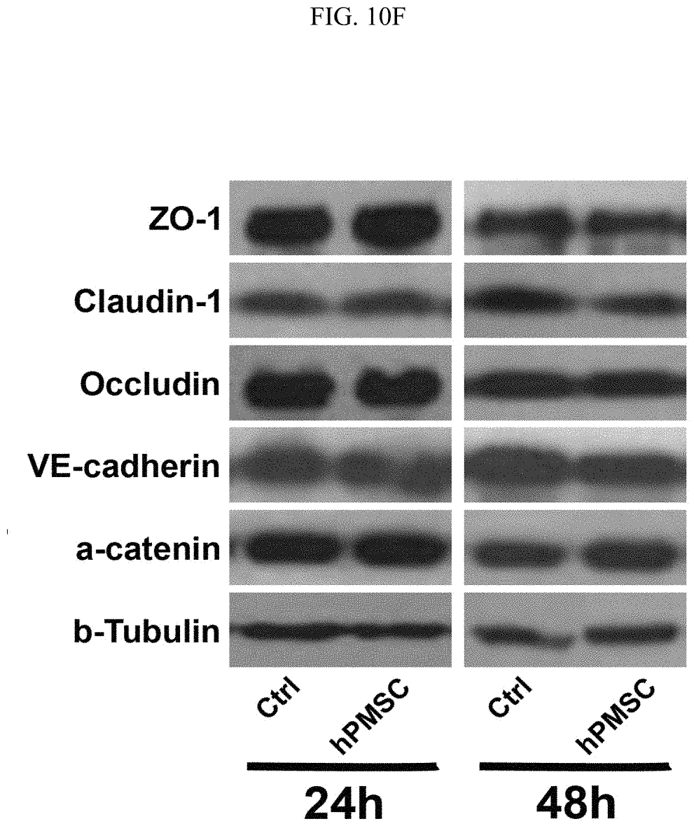

Turning now to FIGS. 1A-18C, a brief description concerning the various components of the present invention will now be briefly discussed.

The presently disclosed invention is a change in the culture conditions used to prepare stem cells (human placenta derived, autologous or other stem cell origin) by which a stem cell culture becomes super-therapeutic by virtue of the stem cells now enhanced capacity to form beneficial exosome/microparticles, which maintain blood flow and preserve tissue integrity. This involves cholesterol and lipid supplementation which increases the cell content of cholesterol and lipids, making the stem cells able to form an increased number of exosome/microparticles, and become enhanced stem cells. One surprising benefit of enhancing the stem cells is it allows the stem cells to be frozen and stored for therapeutic administration at a later time point, with maintenance of the stem cells' beneficial properties, which is normally lost during the preservation/freezing process. Additionally, these enhanced microparticles exhibit less phosphatidylserine on their surface, which enables the enhanced microparticles to more easily penetrate into tissues and have reduced influence on blood coagulation. This enables the enhanced stem cells and exosome/microparticles derived from them to be administered intravenously as well as intraperitoneally. Additionally, and importantly, the enhancing process makes it possible to use only 20% of the amount of stem cells to provide complete protection against stroke injury in the middle cerebral artery occlusion model of stroke (MCAO) compared to non-enhanced or unmodified stem cells—in a process described below. The inventors found that administration of 500,000 unmodified stem cells into the peritoneum was significantly protective in stroke models, but that 100,000 of these unmodified stem cells was not protective against a stroke when given intraperitoneally. By comparison, the inventors were able to obtain significant protection against stroke injury when just 100,000 cholesterol/lipid supplemented enhanced stem cells were administered (the inventors' lab describe as the supplemented cells as “activated” or enhanced). Important to the protection is the maintenance of blood flow to the brain at 24 hours, which is lost in normal stroke affected brains. Additionally, behavioral scores are also preserved when enhanced stem cells (at a lower dose than required for unmodified stem cells) are administered after the stroke.

Protection Against Stroke Injury in the MCAO Model of Stroke with Non-Enhanced Stem Cells:

Post-stroke injury is highly complex involving diverse cell types and mechanisms. So far, the failure of stroke treatments targeting individual molecular pathways may reflect the need to simultaneously suppress many different destructive processes which are active following stroke. Stem cell-based therapeutic approaches have great potential to simultaneously limit multiple factors contributing to stroke injury.

However, it is critical to optimize stem cell delivery methods in order to minimize the cell therapy related complications. Most recently, cell therapy for stroke has been attempted from intravenous, intraarterial, intrathecal, and intracranial routes, each with their own unique pitfalls, particularly safety and intensification of coagulation risk. Here, intraperitoneal injection of placenta stem cells is introduced as an alternative approach to overcome the complications associated with intravenous and intracranial injection of stem cells which maintains normal blood flow distribution in the post-stroke brain.

Ischemic stroke remains the leading cause of permanent neurological disability and is the 3rd leading cause of death in the US. Despite thrombolytic therapy for stroke, ischemia/reperfusion (I/R) injury still deranges normal blood flow regulation, disrupts the blood brain barrier (BBB) and leads to long-term tissue destruction and impairment of cerebral functioning. Besides long-term trans-differentiation into neural cells, benefits of stem cell therapy (SCT) in stroke include secretion of protective factors, which partly reflects extracellular vesicle (EVs) release by stem cells, however, the mechanism(s) by which stem cells/EVs limit stroke injury have yet to be defined. The inventors evaluated human placenta mesenchymal stem cells (hPMSC) as a form of SCT in an acute experimental model of ischemic stroke. Using the transient middle cerebral artery occlusion (MCAO)/reperfusion model, the inventors found that intraperitoneal administration of hPMSCs or intravenous EVs at the time of reperfusion significantly protected the ipsilateral brain hemisphere against ischemic injury. This protection was attended by significant greater restoration and maintenance of normal blood flow to brain post-MCAO. The inventors also found that EVs derived from hPMSC promote paracrine-based protection of hPMSCs in the MCAO model in a cholesterol/lipid-dependent manner. The inventors conclude benefits of hPMSC administration in stroke are multifactorial involving preservation of brain perfusion, BBB integrity ultimately shielding neurons from ischemic injury in stroke.

In the US, stroke remains the leading cause of neurologically-mediated disability, and the 3rd leading cause of mortality in adults1 with stroke incidence and occurrence increasing proportionately with aging in both developed and developing nations. Thromboembolic/ischemic strokes account for up to 85% of stroke incidence, while hypertensive or vessel wall pathology hemorrhage-associated strokes accounts for up to 15%. Ischemic strokes provoke an acute and progressive destruction of neurons, astroglia and oligodendroglia with disruption of cortical synaptic structure Continuity of cerebral blood flow (CBF) is critical to maintain brain function with several protective auto-regulatory mechanisms ensuring adequate perfusion to cerebral arteries under normal conditions. Because of the enormous cerebral energy demand and tremendous susceptibility, it is critical to fully restore CBF during stroke. In fact, the only clinical treatment that has been proven to reduce brain damage after stroke is tissue plasminogen activator (t-PA), an enzyme which converts plasminogen to plasm in that dissolves emboli and thrombi, thereby restoring CBF. However, tPA is only effective in stroke if administered within 4-5 hours after the onset of ischemia, and paradoxically, the act of restoring local blood perfusion often triggers reperfusion injury that intensifies stroke severity. Other options for stroke include prophylactic anti-coagulants e.g. heparin which can limit ischemic strokes via inhibition of clot formation and anti-platelet drugs, such as aspirin and clopidogrel that block platelet aggregation leading to formation of thromboemboli; both of these are mainly used to reduce the risk of secondary strokes.

Following ischemia/reperfusion (IRI), several events contribute to stroke injury including depletion of energy and oxygen supplies, inflammatory infiltration of neutrophils and macrophages into brain tissue, impairment of the ‘blood brain barrier’ (BBB) and disturbed vasoregulation leading to irreversible brain injury and cognitive dysfunction. Therefore, it is essential to develop newer and more highly effective stroke therapies which can protect neural tissues and the BBB against initial IRI events and allow functional recovery.

In this study, the inventors tested the therapeutic potential of human placenta derived mesenchymal stem cells (hPMSCs) in the murine MCAO ischemic stroke model. hPMSCs were chosen because they represent a safe, accessible, abundant and highly effective form of SCT therapy, that is inexpensive and free of ethical concerns.

Historically, stem cells have long been assumed to provide benefit in stroke by engrafting within the post-stroke brain where they might trans-differentiate into cells which restitute damaged tissue. However, stem cells have also been proposed to protect brain tissue through paracrine signaling which may limit acute brain IRI via barrier-stabilization and suppression of leukocyte adhesion/extravasation mediated tissue injury. The inventors have observed that least some of these paracrine benefits of SCT are being mediated by extracellular vesicles (EVs) released by stem cells. However, before the inventors' study, the mechanisms by which EVs derived from hPMSCs protect the brain against ischemic stroke were still unclear. Here, the inventors demonstrate that hPMSC-derived EVs restore and maintain brain blood flow following ischemic stroke, to maintain BBB integrity and protect the brain against propagation of tissue injury.

The inventors used the well-established Koizumi method of the murine middle cerebral artery occlusion (MCAO) model to monitor the size of the infarction area, BBB permeability and blood perfusion in the brains of mice with and without SCT using hPMSC and hPMSC/EVs. Importantly, using the inventors' post-stroke treatment approach, the inventors administered the hPMSC/EVs after reperfusion. The inventors found that intraperitoneal (IP) administration of hPMSCs at the time of reperfusion in the inventors' MCAO model produced remarkable and highly significant preservation of ipsilateral hemispheric blood flow, far beyond what could have been anticipated, tissue structure and significantly greater neurological recovery following stroke compared to untreated MCAO. Strikingly, based on several treatments which control EVs, these benefits now appear to reflect effects of EVs released from hPMSCs. Specifically, this benefit appears to be cholesterol-dependent and related to changes in surface presentation of phosphatidylserine/Annexin V binding. The inventors hereby disclose that hPMSC- and hPMSC/EVs based stroke therapy represent an important methodology for maintaining cerebrovascular perfusion and survival.

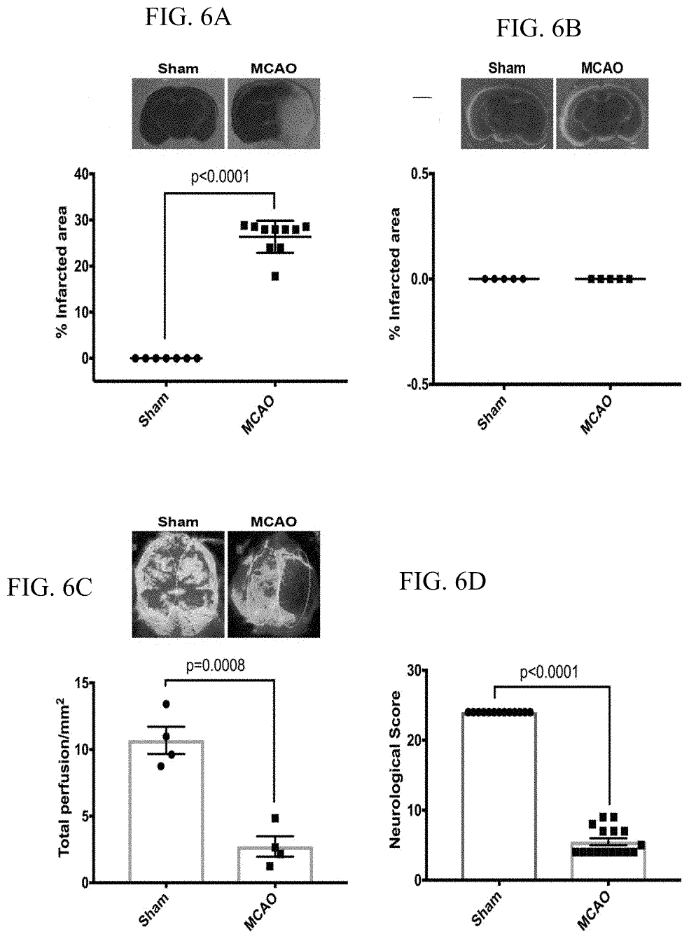

MCAO model of stroke. The inventors used the Koizumi method of MCAO model of stroke to induce occlusive-ischemic injury within one brain hemisphere (ipsilateral hemisphere), by occluding the right middle cerebral artery via introduction of a 6-0 nanofilament for 1 hour, which is the most commonly employed time duration to achieve an 88-100% success rate of infarction. Duration of reperfusion is an essential factor influencing the pathophysiology and outcome in the MCAO model. By TTC staining the inventors found that the infarcted area was grossly apparent in the ipsilateral hemisphere at 24 hours after reperfusion (26.35±1.10, vs. sham group (0±0), p<0.000; Student t-test) (TTC staining; FIG. 6A). However and critically, this effect was not observed in mice with MCAO at 4 hours after reperfusion (FIG. 6B). Thereafter, the inventors continued the inventors' experiments using 1 hour ischemia and 24 hours reperfusion in the MCAO model. Reductions in brain blood flow following MCAO impair motor function and survival. In agreement with this, the inventors demonstrated a significant decrease in blood flow from 10.69±1.017 in sham to 2.723±0.764 in the MCAO group (p<0.0001; Student t-test) in the ipsilateral hemisphere (FIG. 6C) which was accompanied by a substantial decrease in neurological scores 5.0±0.59 in MCAO (impairment of motor function) compared to 24.0±0 in sham mice (p<0.0001; Student t-test) (FIG. 6D).

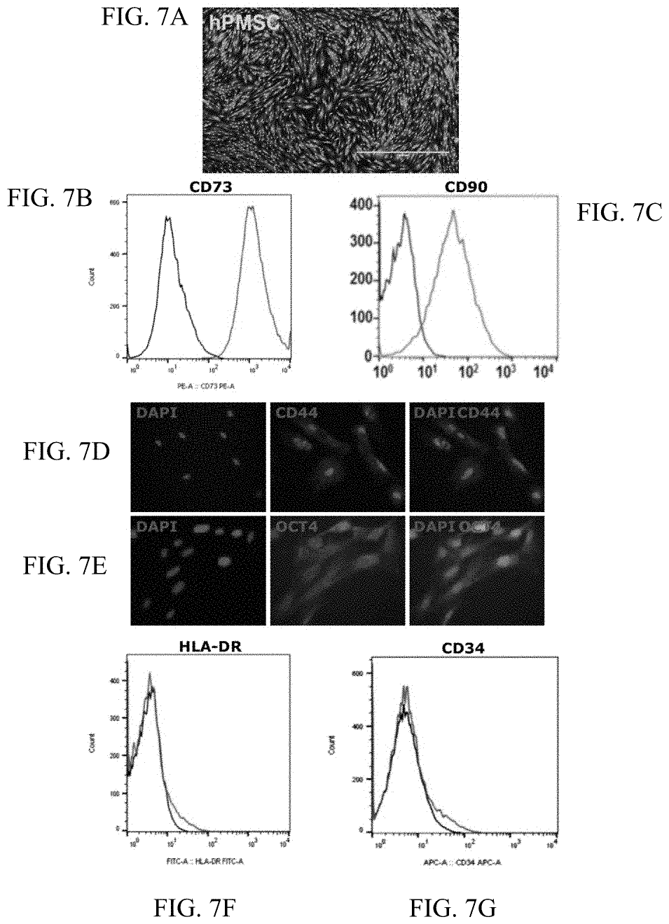

Characteristics of Human Placenta-derived Mesenchymal Stem Cells (hPMSCs). In culture, hPMSCs are plastic adherent cells that are “spindle” shaped, and exhibit fibroblast-like properties e.g. they exhibit robust in vitro expansion and short doubling times (FIG. 7A). The inventors have validated the ‘stemness’ of hPMSCs by measuring the expression of the classical MSC markers CD73, CD90, CD44 and Oct4 using FACS analysis and immunostaining (FIG. 7B-E). hPMSC did not express high levels of HLA-DR, or the hematopoietic lineage marker (CD34) (FIG. 7F-G), which is the major criteria to define MSCs.

SCT is currently being intensively evaluated as highly promising and powerful approach for improving clinical outcomes in stroke. However, SCT has still has encountered several noteworthy complications which have limited their clinical application. For example, in the inventors' pilot studies, the inventors found that following intraperitoneal (IP) injection of SC, >85% of the mice survived the MCAO protocol, comparable to the survival rate observed in the inventors' MCAO treated not injected with stem cells. By comparison, IV injection of stem cells was highly (˜90%) lethal. Irrespective of how scrupulously stem cells were prepared, varied in dosing (from 1-2×10\hat{ }5 cells in 100/200 μl HBSS) or administered by different anatomic routes (e.g., femoral vein, tail vein, carotid artery, and retro-orbital), survival rates >12% could not be obtained. In the inventors' hands IV SCT treatments all showed nearly immediate lethality after administration. Therefore, in the inventors' studies the IV route of stem cell administration was abandoned early on in favor of IP delivery.

IP injection of hPMSCs at the time of reperfusion significantly preserves the ipsilateral hemisphere viability in the MCAO model. The inventors next evaluated whether intraperitoneal (IP) injection of hPMSCs could protect the brain against extensive tissue injury in the inventors' MCAO model. Strikingly, the inventors found that IP administration of 5×10\hat{ }5 hPMSCs at the time of reperfusion (following 1 hour ischemia) shockingly and very significantly reduced ischemic injury (1.827±0.75, p=0.0001, one-way analysis of variance (ANOVA) with Bonferroni post-hoc test), close to levels observed in sham (0±0) animals compared to MCAO alone without SCT (25.84±1.76) (FIG. 1A).

IP injection of hPMSCs at the time of reperfusion significantly preserves ipsilateral hemisphere perfusion at 24 hours in the MCAO model. In order to determine the extent of hPMSCs ability to preserve cerebral blood flow at 24 hours after IP injection of hPMSCs into MCAO treated mice, the inventors measured cerebral perfusion using Laser Speckle Contrast Imaging. The inventors found that mice subjected to MCAO and then treated with hPMSC exhibited a remarkable and significant cumulative preservation of blood flow (7.23±0.57) within the post-MCAO brains compared to untreated post-MCAO brains (3.68±1.12) (p=0.01, one-way ANOVA; FIG. 1B). While the post-stroke decrease in cerebral blood flow in untreated MCAO was more severe in the ipsilateral (infarcted) side (0.31±0.01) compared to the contralateral hemisphere (0.68±0.01), hPMSCs treatment also preserved normal blood flow in both ipsilateral (0.42±0.03) and contralateral (0.57±0.03) hemispheres (FIG. 1C, D), indicating a stroke mediated redistribution of brain blood flow as well as a preservation of blood flow associated with maintenance of post-MCAO tissue survival in the hPMSC-treated group. Consistent with abundant evidence showing that reduced brain blood flow after MCAO chronically impairs neural function and survival, the inventors also observed that neurological function was significantly conserved in MCAO mice treated with IP administered hPMSCs (18.38±1.01) compared to the untreated MCAO group (5.87±0.83) (p=0.0001, two-tailed Student t-test; FIG. 1E).

The destruction of neurons is another hallmark of ischemic stroke injury which may be improved by SCT treatment. In order to visualize and differentiate between viable neurons, degenerating neurons and glial cells in the striatal areas of both ipsilateral and contralateral hemispheres, the inventors performed modified Nissl histological staining (FIG. 1F-I). Nissl staining revealed that numbers of viable neurons decreased in the ipsilateral hemisphere (16.75±7.12) in the MCAO group compared to the contralateral side; this deterioration was significantly (p=0.02, Student t-test) prevented by hPMSC-treatment in the MCAO group (191.5±43.91) (black arrows; FIG. 1F, quantification; FIG. 1G). Contracted neurons with an enlarged intercellular space was considered to be evidence of cellular degeneration in stroke; these figures were also significantly (p=0.04, Student t-test) decreased after hPMSC treatment (146±15.83) compared to the untreated MCAO alone group (208.3±18.81) (black arrows head; FIG. 1F, quantification; FIG. 1H). Many unstained (‘spongiform’) regions were also observed in MCAO treated brains; this appearance was not detected in any hPMSC-treated MCAO brains (white arrows; FIG. 1F). The increased numbers of glial cells (69.75±12.36) in the ipsilateral hemisphere of MCAO treated mice were also significantly (p=0.02, Student t-test) reduced by administration of hPMSC in MCAO (39±6.86) (black asterisk; FIG. 1F, quantification; FIG. 1I).

Several inflammatory mechanisms and penetration of immune cells into the previously ischemic brain are believed to contribute to secondary brain damage following stroke. Early activation of microglia, the resident immune cells of the CNS, is a key neuroimmunological responses seen in response to a wide variety of pathological stimuli, (trauma, inflammation, degeneration, and ischemia). Ionized calcium binding adaptor protein (Iba-1), a calcium binding protein, is specifically mobilized in microglia after inflammation and plays an important role in microglial regulation and activation. To evaluate microglial activation, the inventors performed immunohistochemical staining of brain tissue samples with anti-Iba-1 antibody (FIG. 1J-L). The inventors observed that numbers of microglia (Iba-1⁺) in the ipsilateral hemisphere of hPMSC-treated MCAO mice was similar to that in the MCAO group, 153±5.33 and 160.8±9.31, respectively (p=0.5, Student t-test) (FIG. 1J, K). Elongated microglial cell bodies observed in the MCAO mice (52.02±3.23) were significantly (p=0.002, Student t-test) reduced by administration of hPMSC (39.84±2.23) (FIG. 1J, L); consistent with hPMSC administration inhibiting microglial activation.

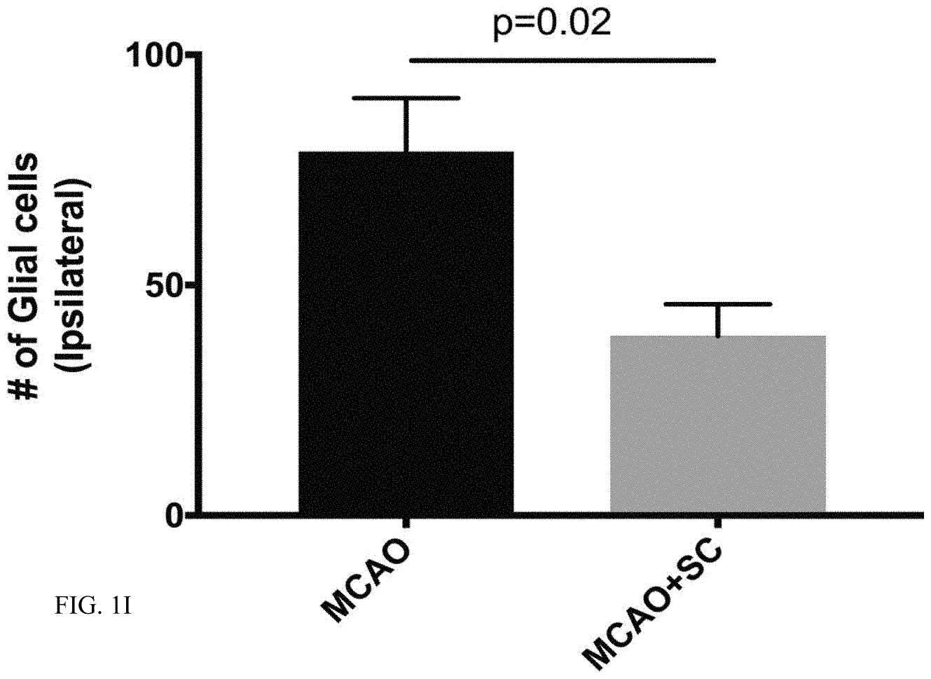

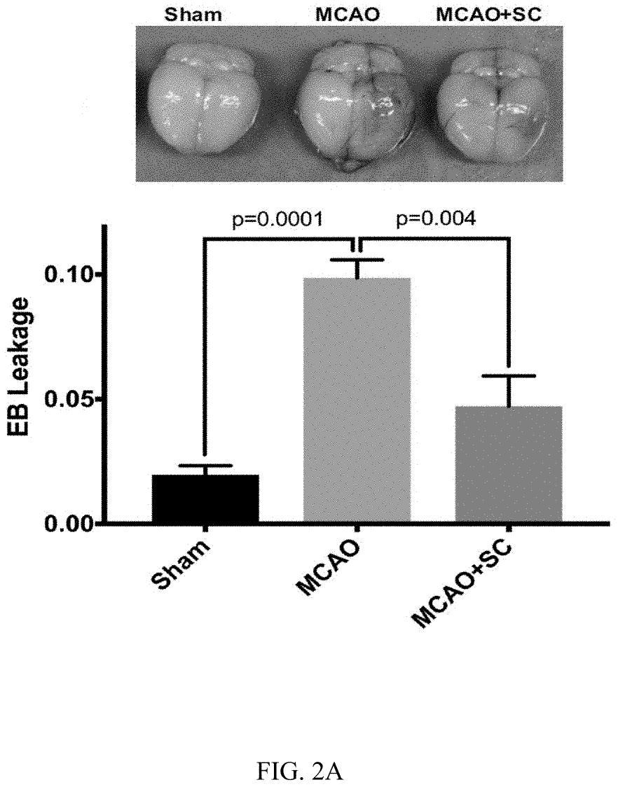

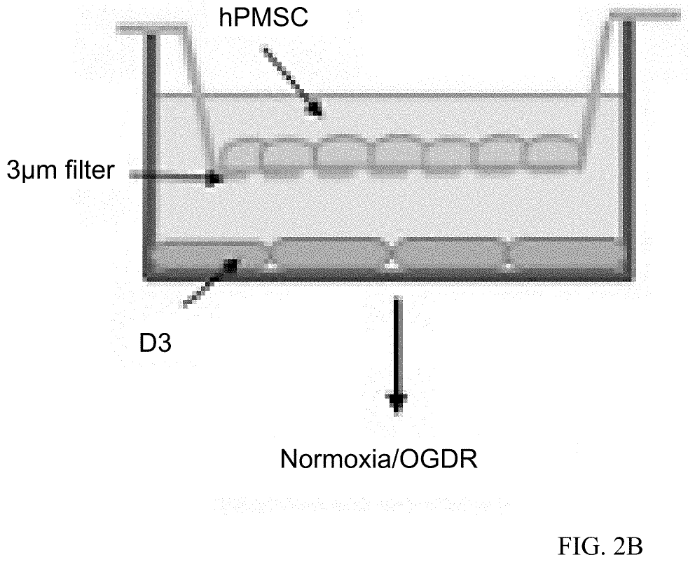

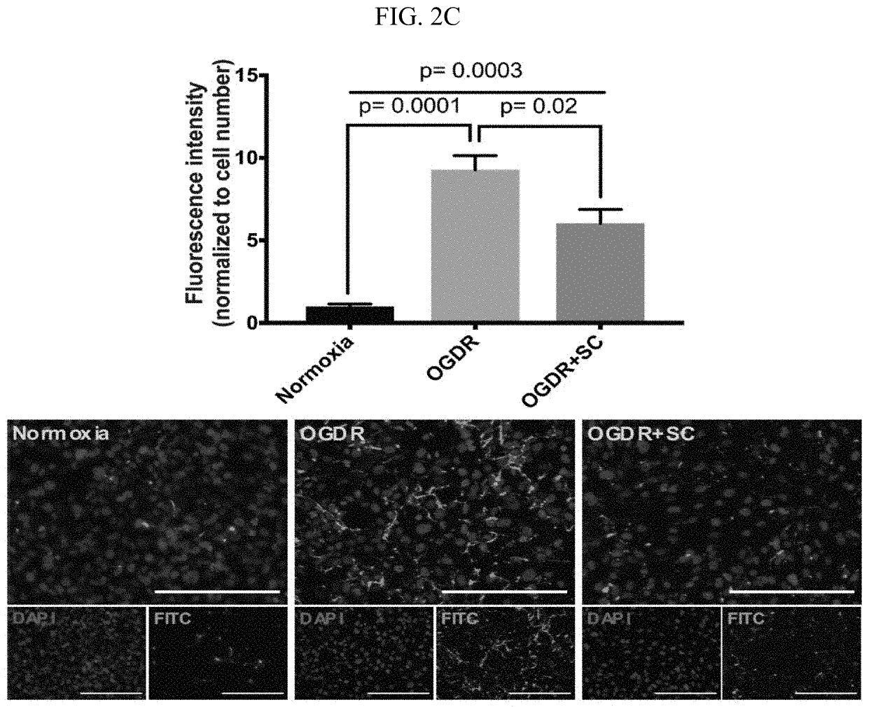

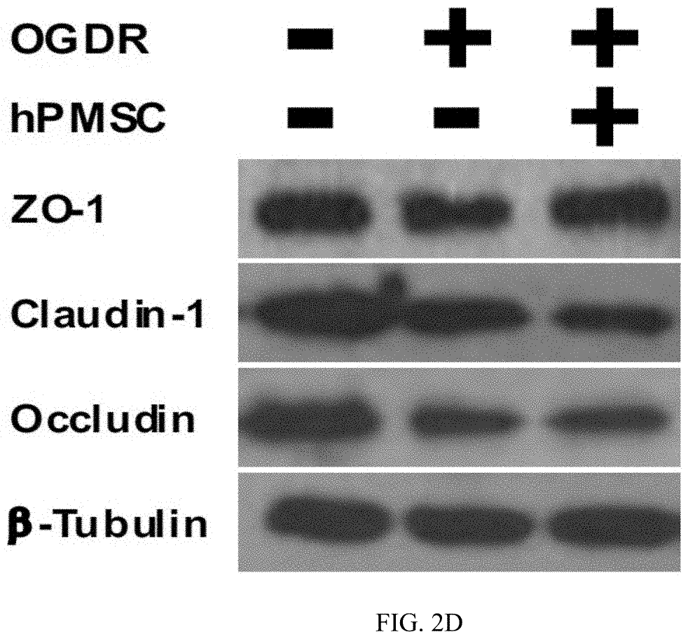

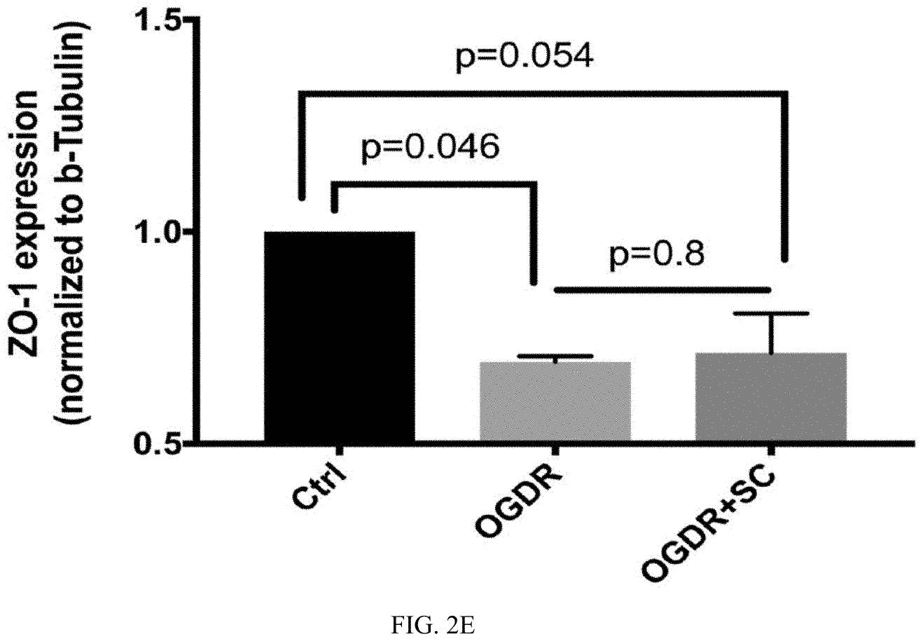

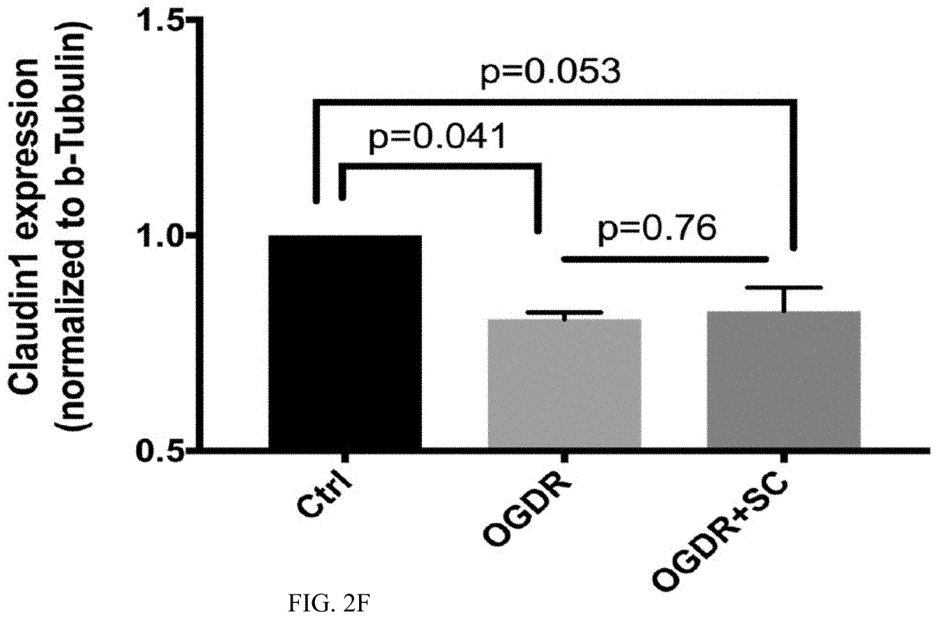

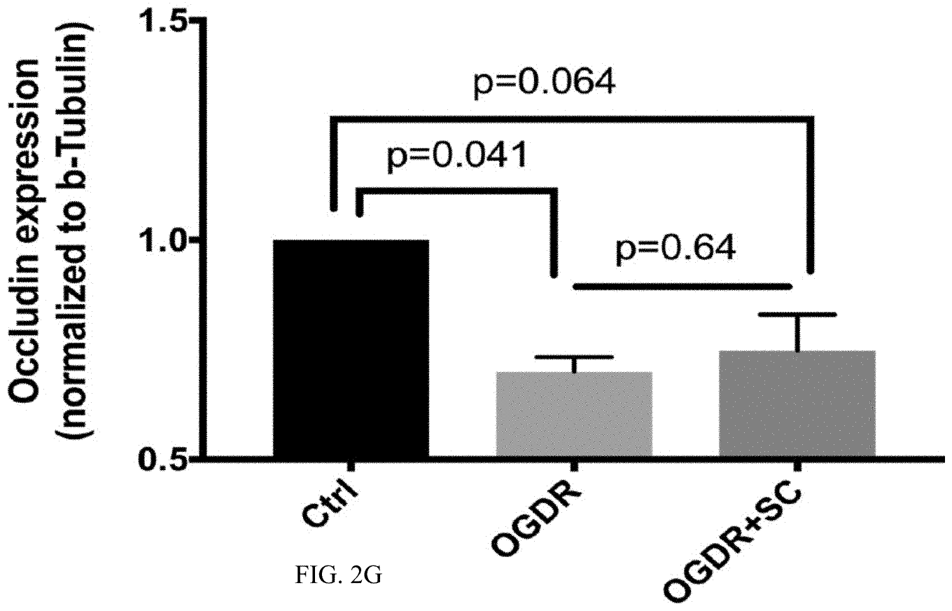

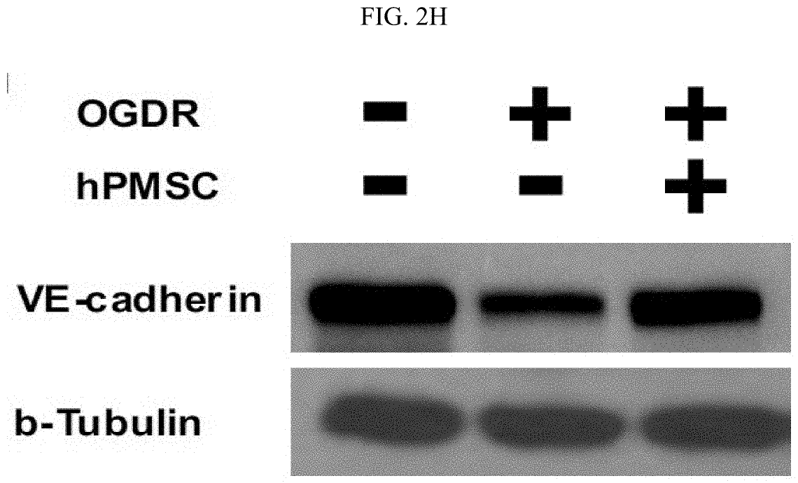

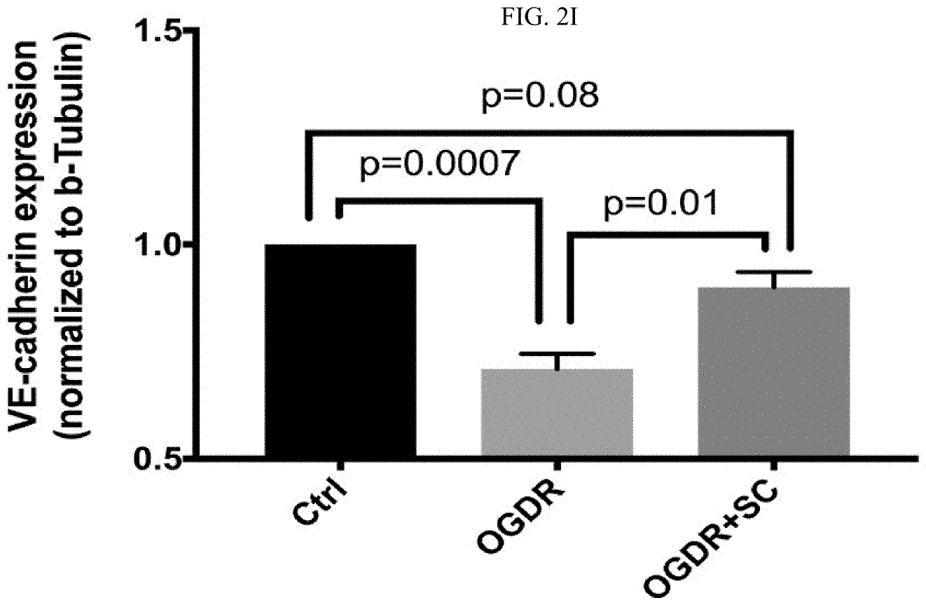

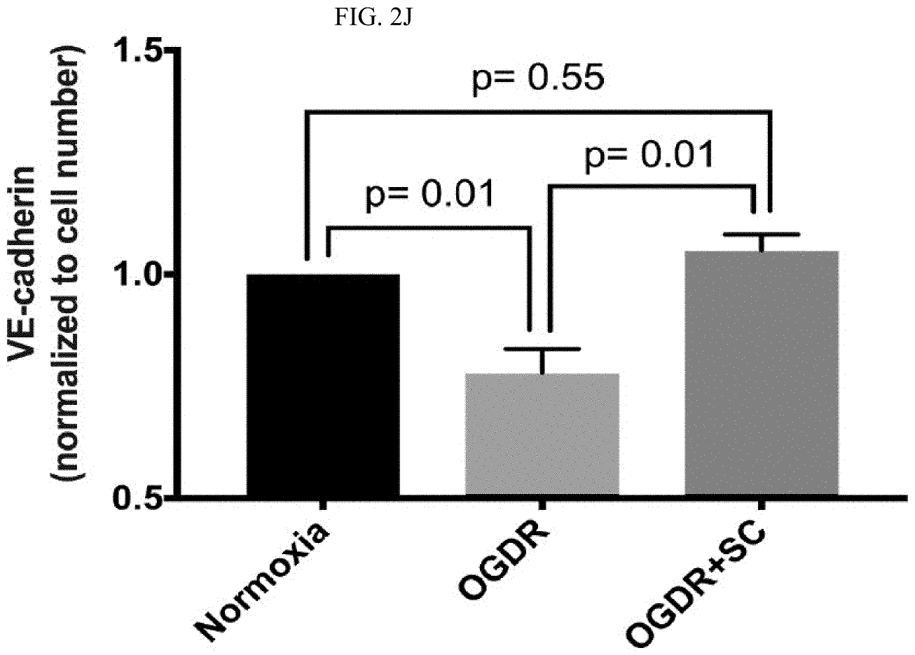

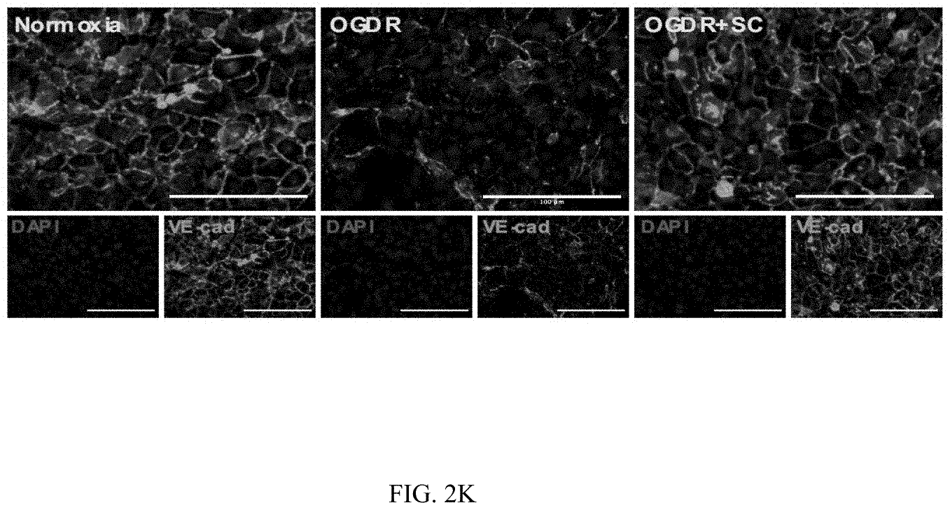

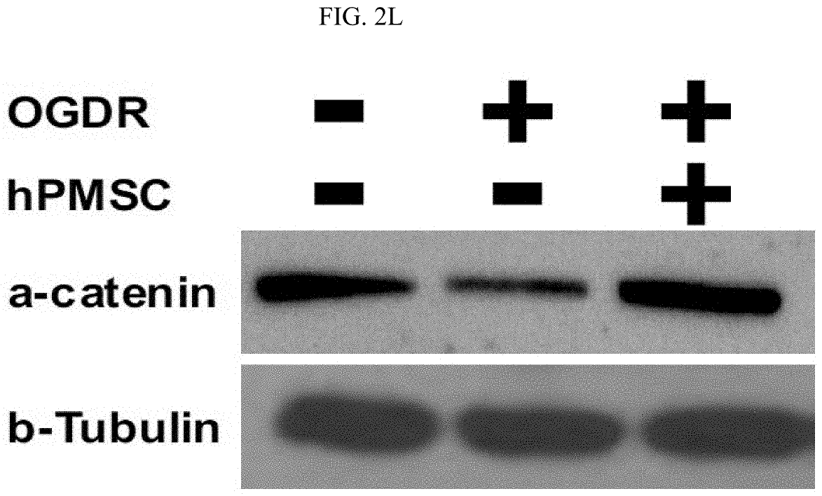

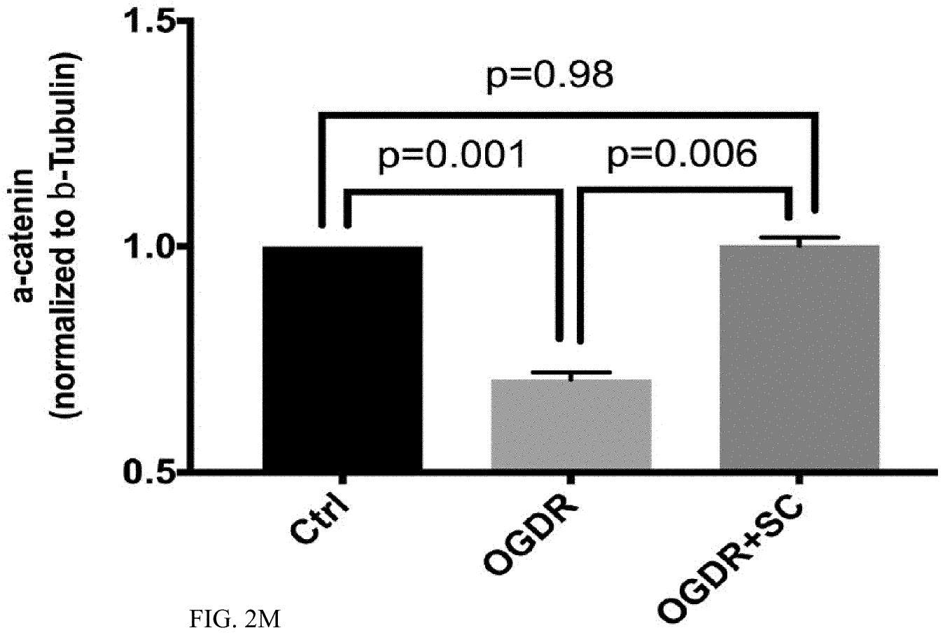

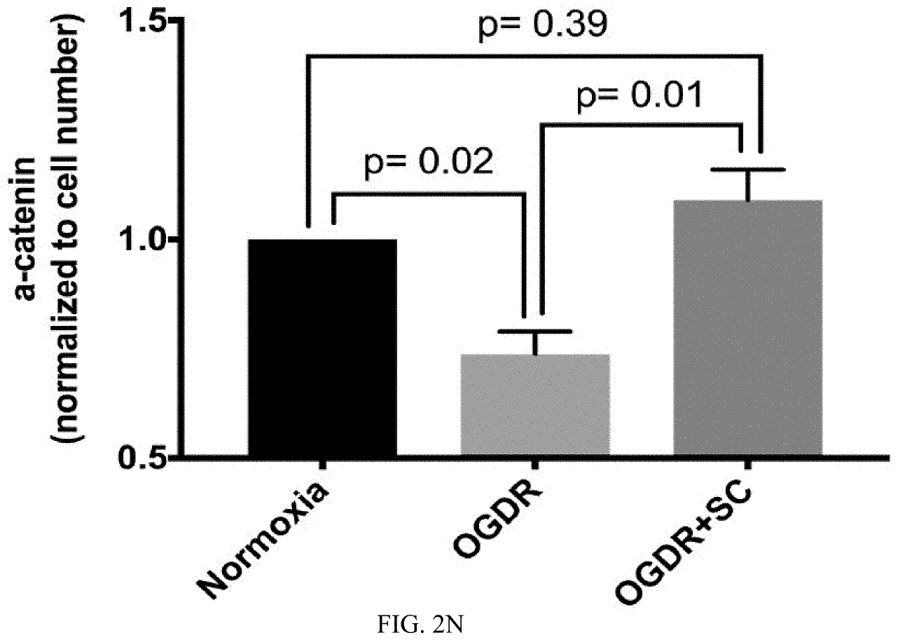

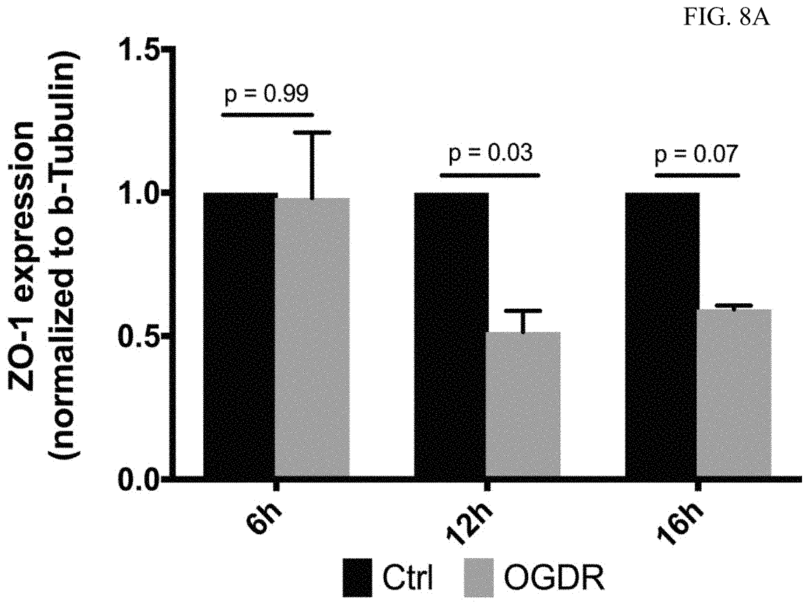

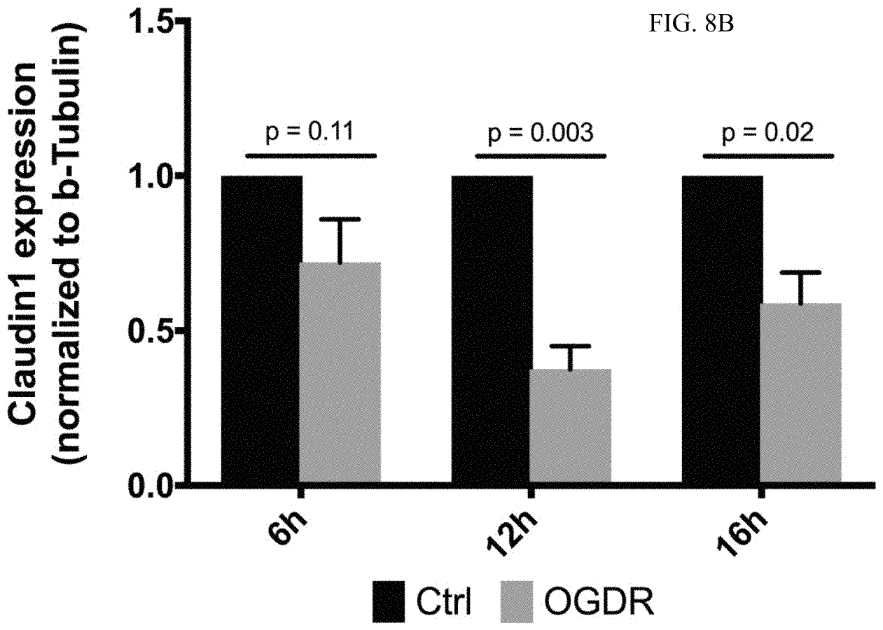

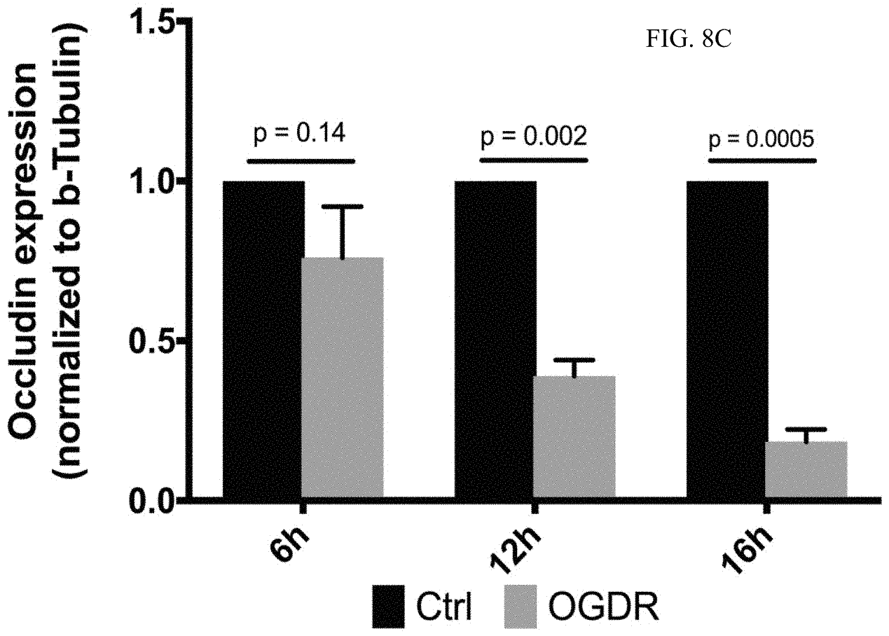

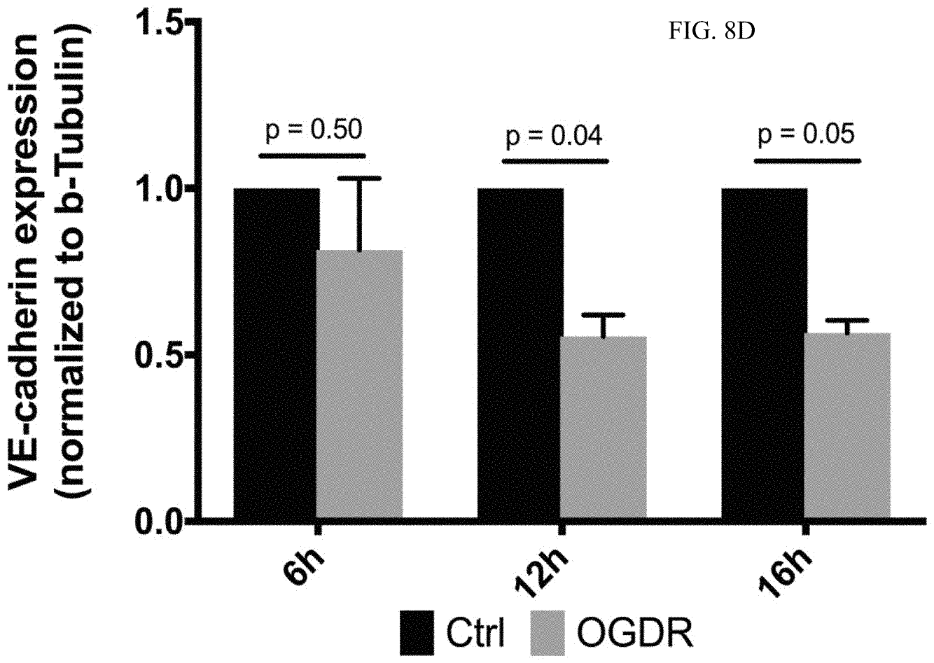

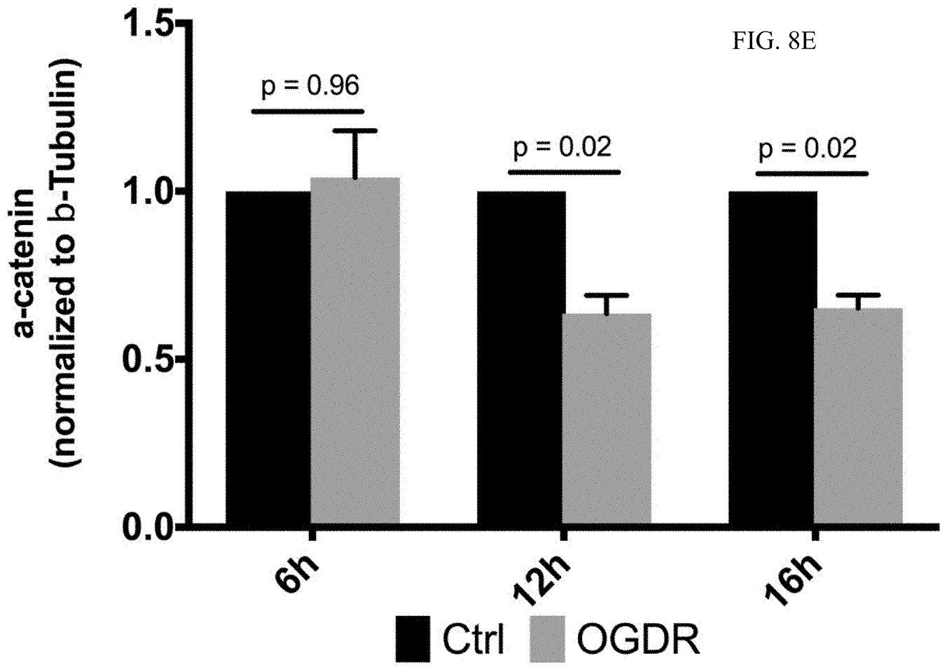

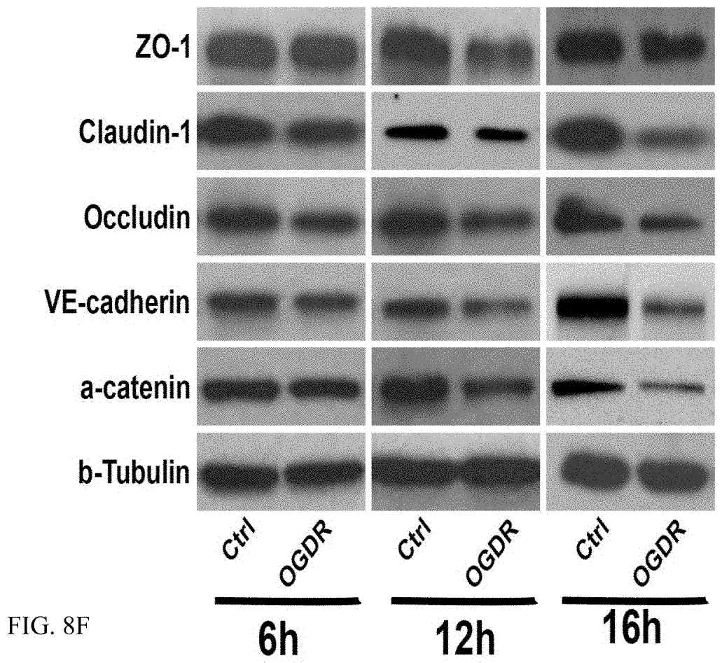

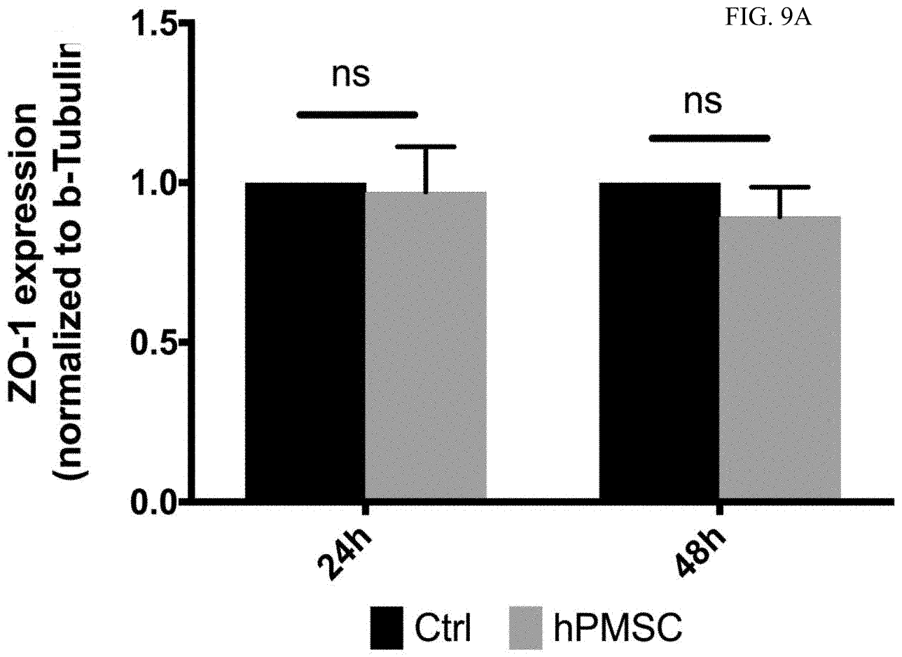

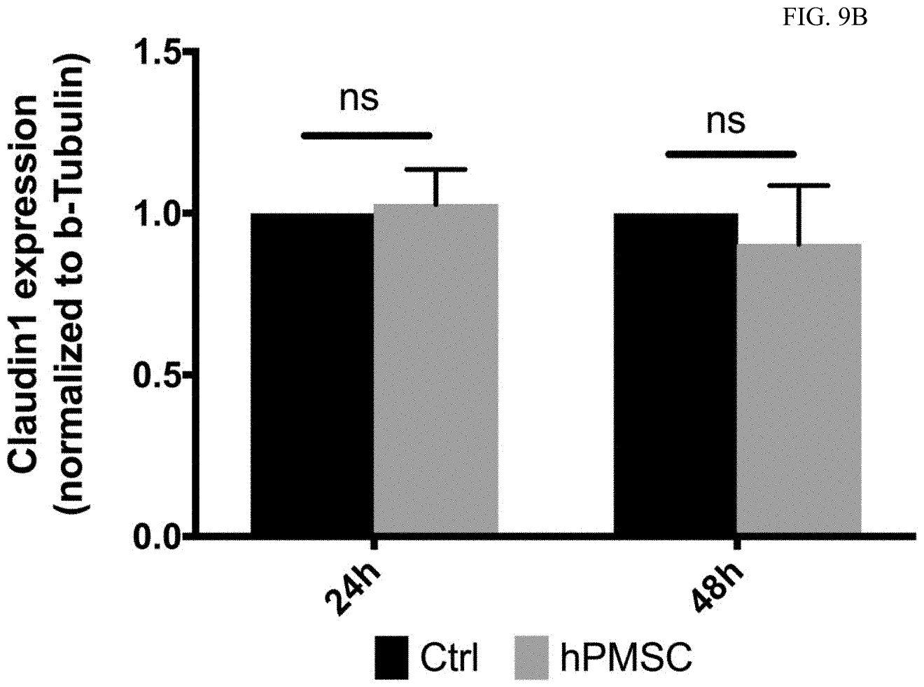

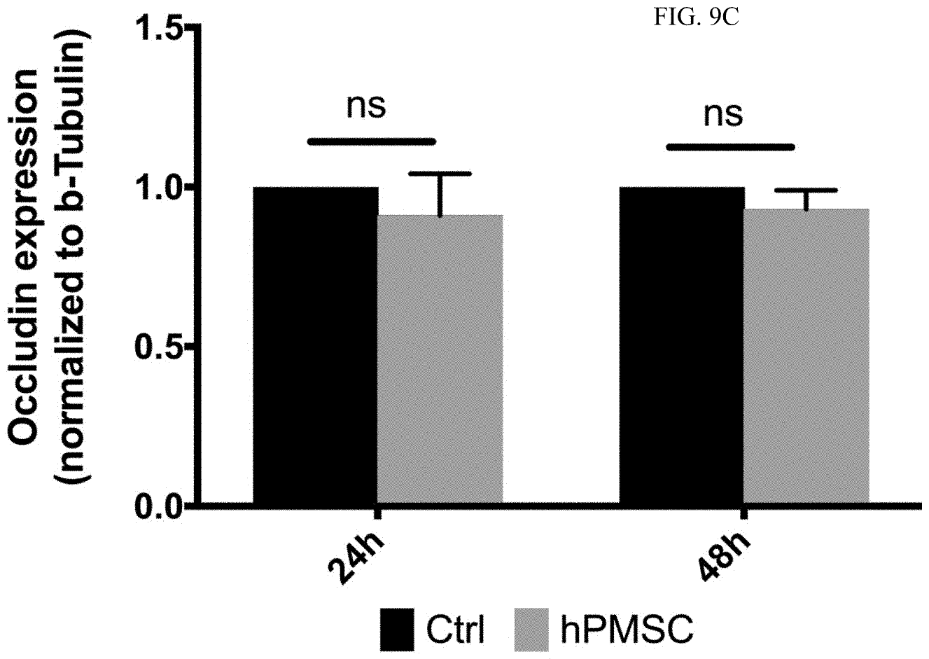

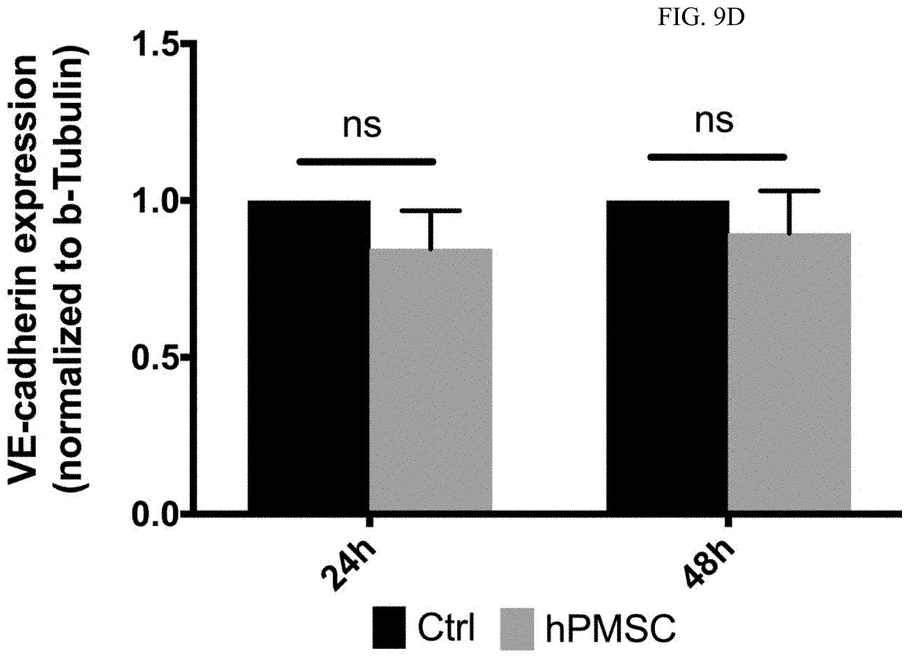

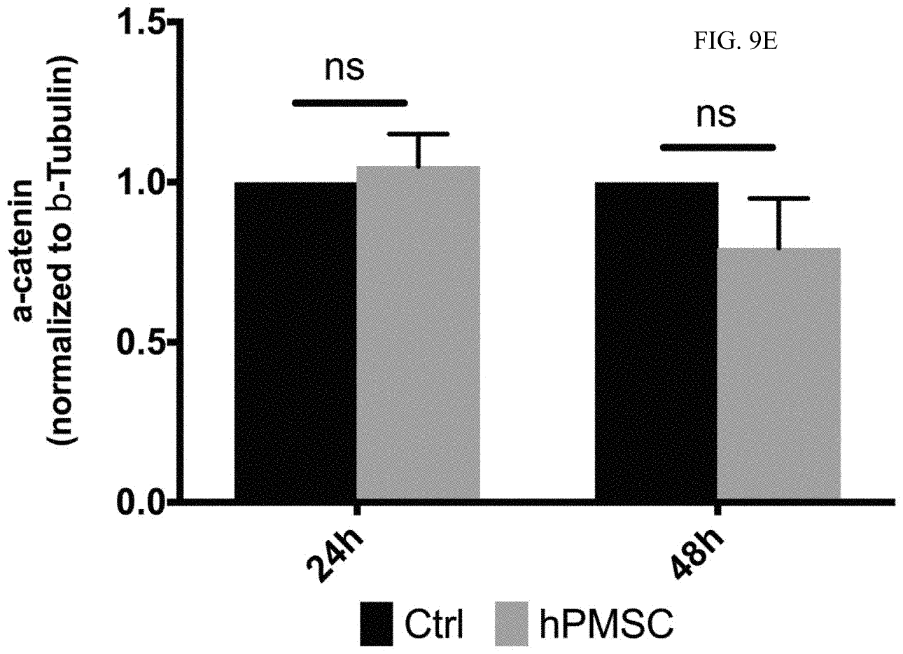

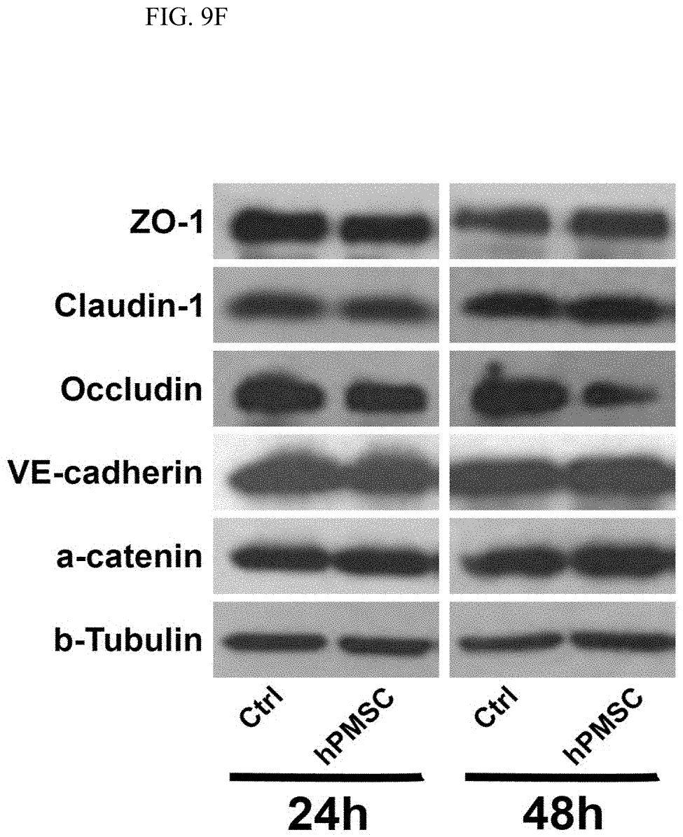

hPMSCs maintain blood brain barrier integrity against MCAO in mice. To characterize changes in BBB integrity in MCAO-stressed brains, Evans blue (EB) vascular permeability analysis was performed. FIG. 2A shows that EB leakage significantly increased in the ipsilateral (right) hemisphere of MCAO group (0.09±0.007 vs. sham, 0.02±0.01, p=0.0001, one-way ANOVA) shown by blue tissue stain (FIG. 2A); indicating that BBB function was lost. In contrast, 24 hours after administration of 5×10⁵ hPMSCs, BBB integrity was maintained as indicated by low EB uptake (0.04±0.01, p<0.0001, Student t-test) in the leakage of EB into the ipsilateral hemisphere compared to MCAO group (FIG. 2A). Oxygen glucose deprivation (OGDR) increases endothelial permeability; similar stresses in stroke may impair BBB function. The inventors therefore investigated whether hPMSCs could maintain the in vitro barrier formed by human brain endothelial cell (hCMEC-D3) against OGDR (FIG. 2B). Using a biotinylated gelatin FITC-avidin permeability assay the inventors found that OGDR significantly increased FITC-avidin permeability (9.26±0.87, p=0.0001, one-way ANOVA; FIG. 2C), and that hPMSCs (via contact-independent co-culture) stabilized hCMEC-D3 barrier integrity against OGDR stress (6.03±0.83, p=0.02, Student t-test; FIG. 2C). The inventors' findings indicate that hPMSCs release soluble factors that improve hCMEC-D3 monolayer barrier function may contribute to protection against injury in MCAO in SCT. The inventors also previously reported that increased endothelial permeability associated with ischemia may reflect alterations in organization of tight/adherens junctional (TJs/AJs) proteins e.g. occludin, claudins, VE-cadherin and catenins. To assess the impact of OGDR on TJs/AJs proteins, the inventors measured TJs/AJs protein expression under OGDR at 6, 12 and 16 h time points. As depicted in FIG. 8A-F, the inventors found significant reductions in the expression of TJ proteins (ZO-1; p=0.03, Claudin-1; p=0.02, Occludin; p=0.0005, two-way ANOVA; FIG. 8A-C) and AJs (VE-cadherin; p=0.05, a-catenin; p=0.02, two-way ANOVA; FIG. 8D-E) after 16 h incubation of hCMEC-D3 monolayers under OGDR stress. The inventors next evaluated whether hPMSCs could prevent this loss of TJs/AJs in hCMEC-D3 monolayers under both normoxia and OGDR. ZO-1 (p=0.8; FIG. 2D,E), claudin-1 (p=0.76; FIG. 2D,F) and occludin (p=0.64; FIG. 2D,G) did not change significantly under OGDR with hPMSCs (Student t-test; FIG. 2D-G) nor normoxia (Student t-test; FIG. 9A-C,F). However, the inventors observed that hCMEC-D3 monolayers (contact independently co-culture w/hPMSCs) expressed significantly more VE-cadherin (p=0.01,; FIG. 2H, I) and α-catenin (p=0.006, Student t-test; FIG. 2L, M) under OGDR conditions but not under normoxia (Student t-test; FIG. 9D-F). Spatial localization of both VE-cadherin (K) and α-catenin (O) of D3 monolayers improved with hPMSC treatment under OGDR stress. Additionally, VE-cadherin (p=0.01; FIG. 2J, K) and α-catenin (p=0.01; FIG. 2N, O) expression increased under OGDR at hCMEC-D3 junctions after hPMSCs treatment. These findings are consistent with hPMSCs releasing soluble factors which enhance and organize endothelial junctions under stressed, but not control, conditions.

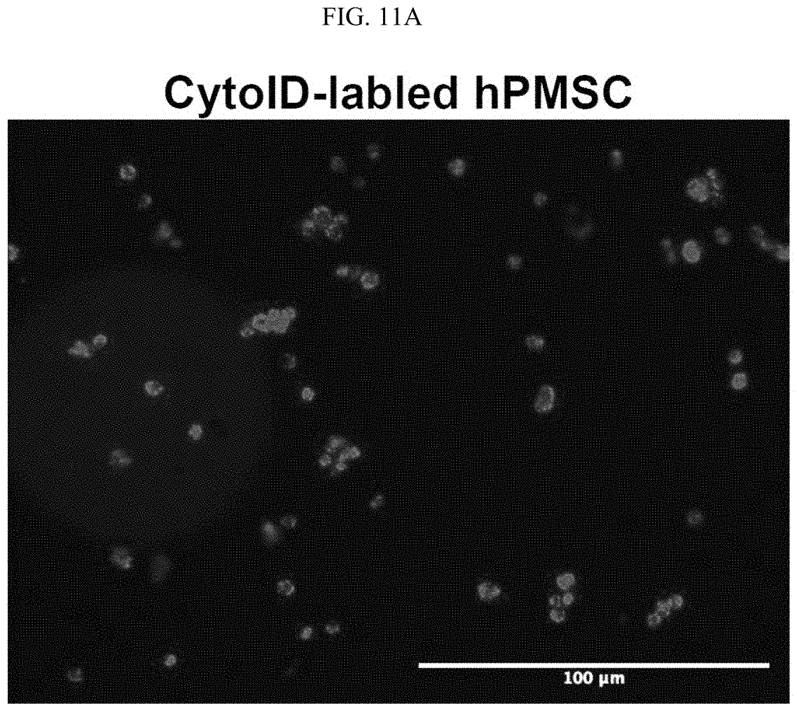

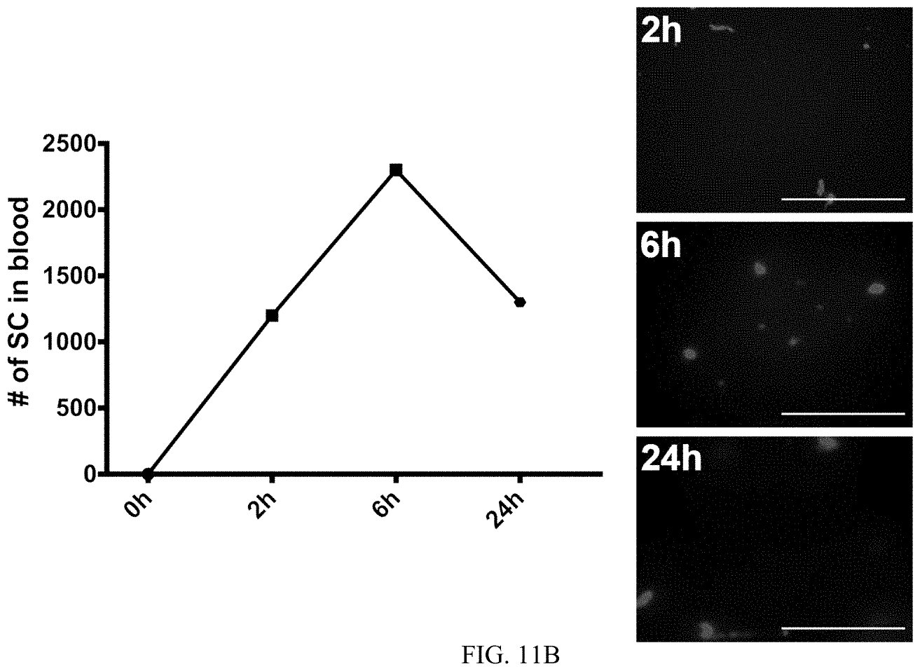

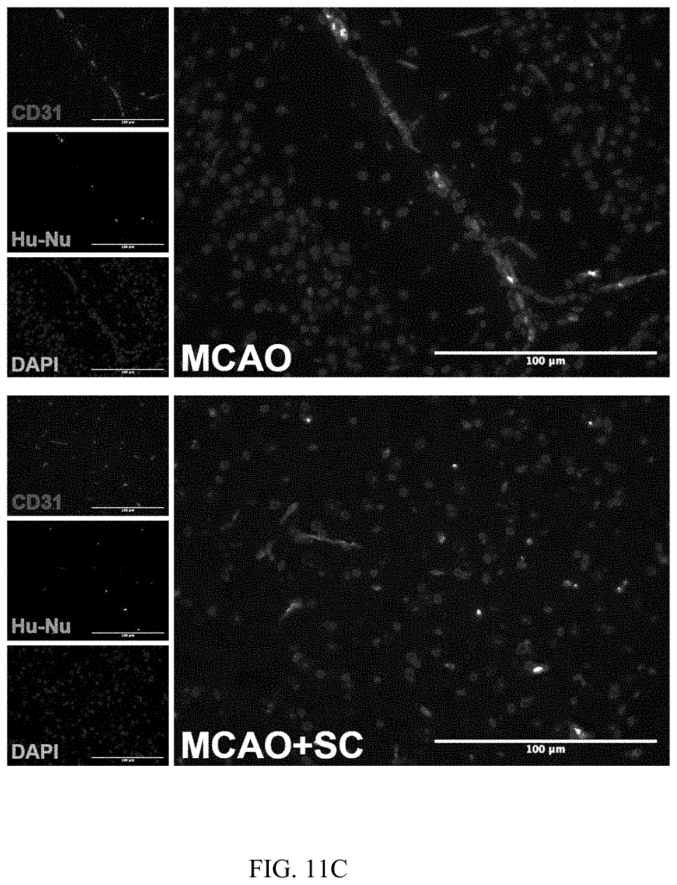

hPMSC-released EVs contribute to SC-based protection in the MCAO model. To investigate mechanisms underlying hPMSCs protection in the MCAO model, CytoID-dye labeled hPMSCs (FIG. 11A) were injected IP into MCAO treatd mice, and blood collected at 2, 6, and 24 h post-injection to determine numbers of circulating hPMSCs in the bloodstream. Interestingly, the inventors found relatively low numbers of circulating hPMSCs (max 2300 cells/ml blood after 6 h; FIG. 11B). The inventors next examined the brain for the presence of hPMSCs using an anti-human nuclear antibody (specifically detects human cells); Mouse CD31 was used as a positive control. The inventors did not detect any hPMSCs penetrating the brain (FIG. 11C), suggesting that beneficial effects of hPMSCs might be mediated by paracrine signaling pathways restricted to the vasculature rather than cell-integration into injured tissues.

Thromboembolic strokes account for 87% of the total stroke incidence and stroke remains the leading cause of neurologically-based morbidity. In ischemic stroke, UR injury at the time of, and following therapeutic restoration of cerebral blood flow often increases intracerebral mobilization of inflammatory mediators that impair BBB, enhance endothelial adhesion molecule expression and perturb normal cerebral blood perfusion, all of which can greatly intensify stroke severity. Following the onset of stroke, pharmaceutical intervention is limited to two classes of FDA-approved drugs: tissue-plasminogen activator (t-PA), which dissolve clots, and anti-platelet therapies e.g. aspirin and clopidogrel. While these treatments can restore blood flow to the brain, they do not prevent the initiation and progression of cerebral reperfusion injury and still carry serious risks for hemorrhage. The lack of highly effective and safe therapies for this critical acute phase of stroke still requires alternative therapeutic approaches to limit stroke injury.

Mesenchymal stem cells (MSCs) can be found in many different tissues and organs from blood, adult and fetal tissues. The minimal defining characteristics for mesenchymal stromal cells are plastic adherence, expression of several ‘cluster of differentiation’ (CD) markers (CD44, CD90, CD73, CD105 and CD166) and capacity to differentiate. The placenta also represents another important, and perhaps more clinically useful source of MSC for stem cell therapy (SCT). Since placentas contain an extremely high density of stem cells, there is no need to undergo invasive procedures encountered with fetal-derived and autologous SCT, and placenta collection has no “ethical” concerns encountered with fetal tissues.

In the inventors' current study, the inventors characterized hPMSCs expression of CD34⁽⁻⁾, CD10⁽⁺⁾, CD200⁽⁺⁾, and CD105⁽⁺⁾ (FIG. 7).

While SCT may be useful in chronic settings, the early and effective limitation of initial stroke injury still represents the greatest opportunity to successfully manage injury and would therefore limit the amount of repair that might be required at later times. At present, however, far less is known about the benefits of acute SCT administration and the basis of protection offered by acute stem cell therapy which may differ from that in chronic SCT. Indeed, the inventors found that IP administration of hPMSCs (5×10⁵ cells in 500 μl HBSS) at the time of reperfusion (following 1 h ischemia) provided extremely potent protection against tissue injury in the MCAO model of stroke (FIG. 1A) with >85% survival. When stem cells were administered intravenously (IV), only a small fraction (1%) actually penetrate to the brain. More importantly, IV administration of SC can too often trigger disastrous intravascular coagulation leading to intense injury or death. Therefore, while SC might have enormous potential, IV administration of stem cells still carries tremendous risks. For example, similar to the inventors' findings (in which ˜12% of mice survived IV injection of SC), there is ˜85% mortality due to pulmonary embolism when IV-SC were administered to otherwise healthy mice. To the inventors' knowledge, the inventors' model shows for the first time that, unlike IV therapy, IP-hPMSC administration is highly effective, apparently safe and very well-tolerated as therapeutic approach in MCAO.

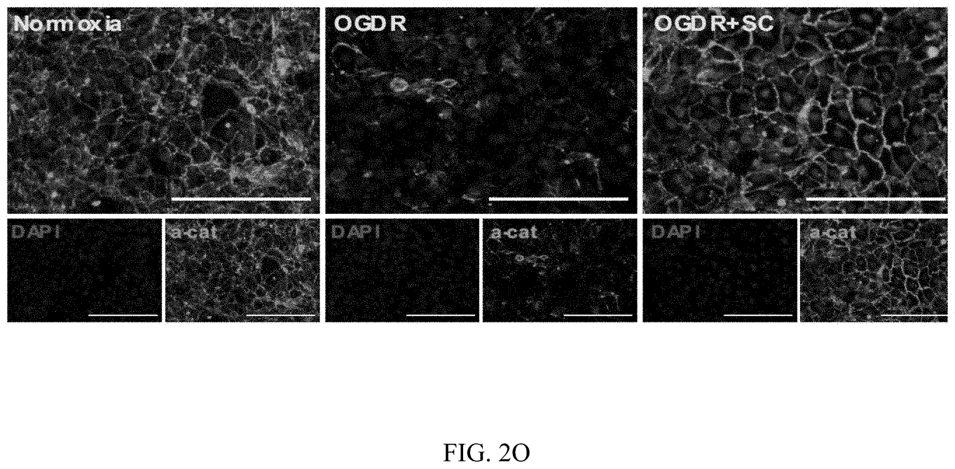

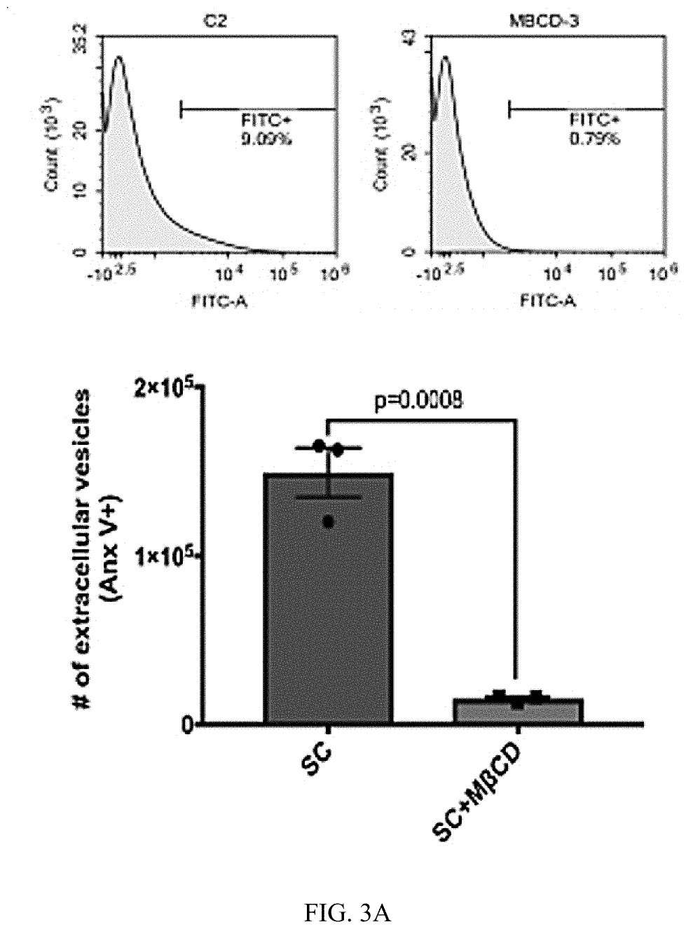

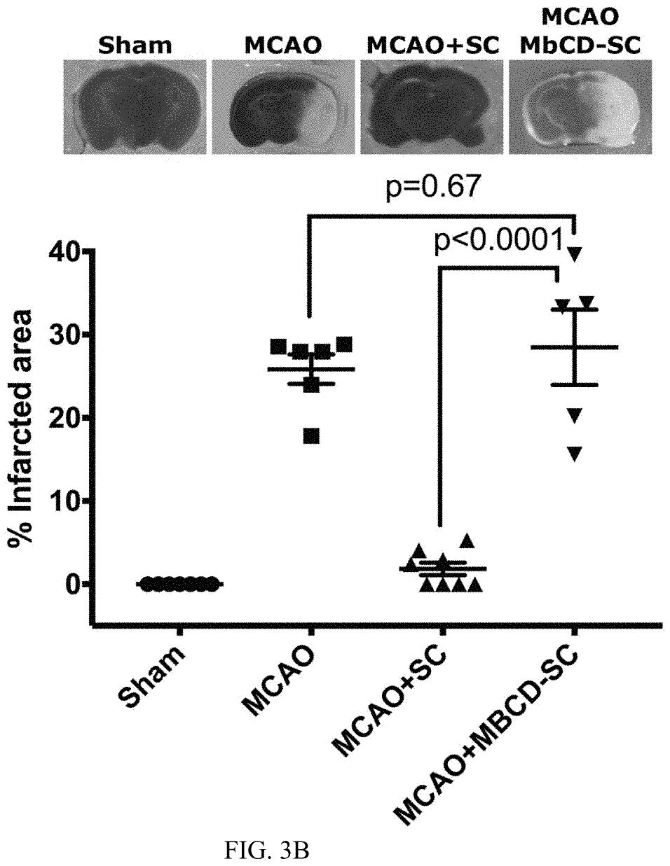

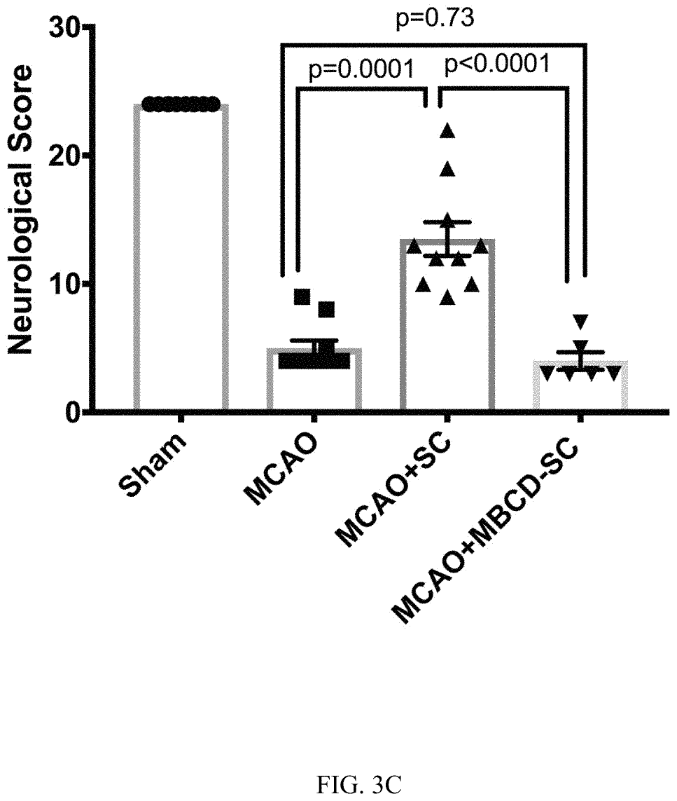

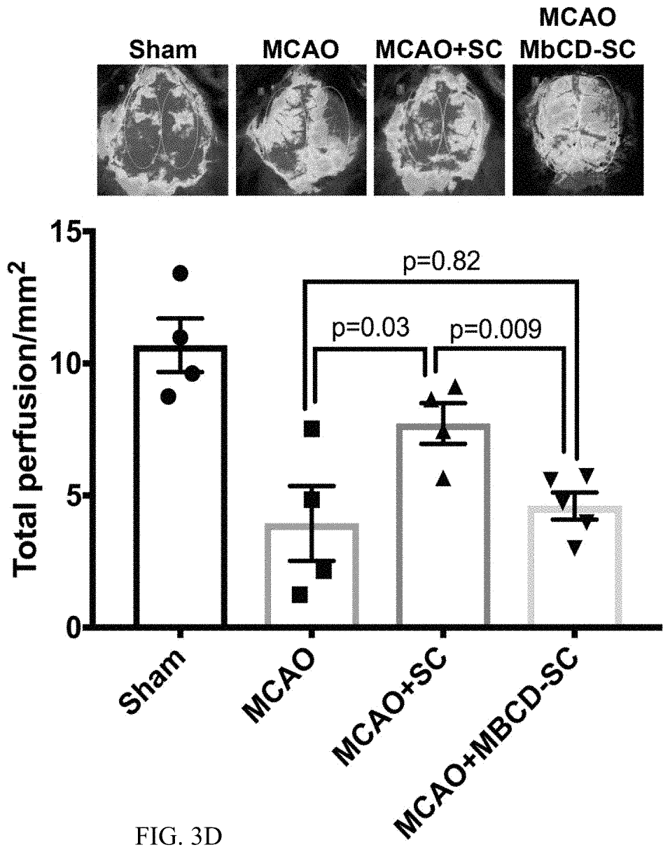

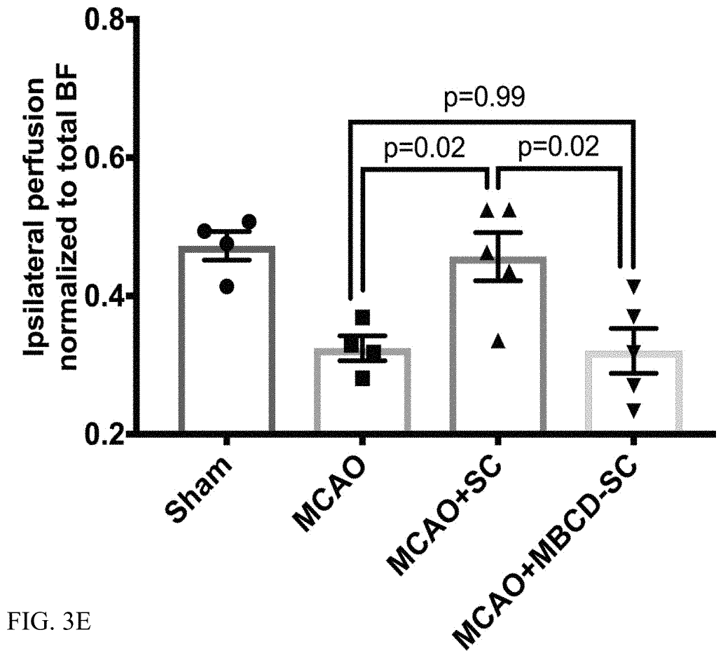

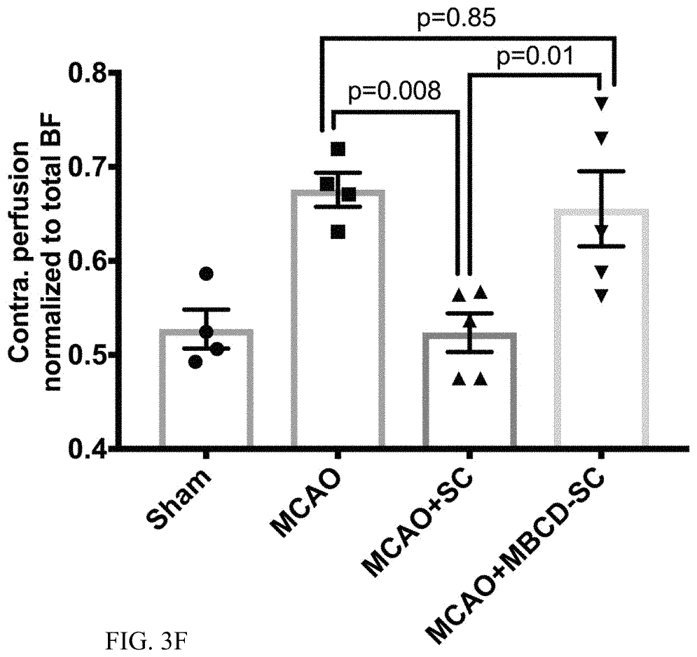

Furthermore, and most strikingly, hPMSC-treated mice subjected to MCAO exhibited significant preservation of blood flow to the ipsilateral hemisphere compared to MCAO-treated brains (FIG. 1B-D). The inventors also found that maintenance of normal blood flow to the ischemic brain hemisphere provided by IP-hPMSC following MCAO was associated with significant neurological protection (FIG. 1E). Neuron survival (shown by Nissl staining) promoted by hPMSCs (FIG. 1F-I) is also improved during recovery. The inventors also observed that following cerebral ischemia, overall blood flow to the brain was reduced post-MCAO, with an apparent shift of the remaining cerebral blood flow to the non-infarcted hemisphere. Therefore, disturbances in normal hemispheric blood flow distribution could have disastrous consequences for both the infarcted and companion hemispheres. Some of the beneficial effects of MSCs may reflect paracrine signaling actions, instead of (or in addition to) differentiation into target tissue types. The inventors have now obtained several lines of evidence consistent with hPMSC protection against MCAO injury being mediated by EVs. For example, the inventors observed a remarkable loss of protection against MCAO injury when hPMSC were pre-treated with MCD (FIG. 3), suggesting that stem cell-derived EVs contribute to paracrine benefits of these cells in stroke.

### MATERIAL AND METHODS. STUDY DESIGN

The objectives of this study were to determine the mechanisms and extent to which hPMSCs protect the brain against acute ischemic injury in vivo, and to characterize barrier-stabilizing and anti-inflammatory effects of hPMSCs in vitro and in vivo. The inventors used middle cerebral artery occlusion (MCAO) as an in vivo model of ischemic stroke in C57BI/6 mice using a 1 hour ischemic period following by 24 hour reperfusion. hPMSC/EVs were injected (IP/IV) at the time of reperfusion to evaluate how hPMSC/EVs can protect against I/R injury induced by MCAO. A sham group was used as controls to ensure that surgery and anesthesia did not contribute to observed results. Cerebral blood flow, infarct size, BBB integrity, and neurological scores were measured in all experimental groups. The inventors also used oxygen glucose deprivation/reperfusion (OGDR) conditions as the inventors' experimental in vitro model of stroke-like stress where hPMSCs were contact independently co-cultured with human brain endothelial cell (hCMEC-D3) to evaluate the protective function of hPMSCs on the in vitro barrier generated by human brain endothelial cell (hCMEC-D3) monolayers under normoxic and OGDR conditions. In general, the inventors used n=4 to 10 mice per group for in vivo experiments and n=3 for in vitro experiments (with three replicates). All experiments were designed with commitment to reduction and replacement to minimize the number of mice used in the study.

In vivo experiments. Animals. All animal protocols were approved by the Louisiana State University Health Sciences Center Institutional Animal Care and Use Committee (IACUC) according to NIH guidelines. The inventors used male C57BI/6J mice (Jackson Laboratories, Bar Harbor, Me.) in all studies at 9-16 weeks of age. Animals were housed in a barrier facility and maintained on a normal diet.

MCAO model. Surgical procedure. Male mice (25-30 g) were anesthetized with ketamine (200 mg/kg i.p.) and xylazine (10 mg/kg i.p.). Once under deep anesthesia, middle cerebral artery occlusion (MCAO) was induced by creating a midline incision at the neck to expose the right carotid bifurcation. The right external carotid artery branch was isolated and ligated and the common carotid artery microclipped to permit creation of a small hole in the middle of the common carotid artery. A silicone-coated 6-0-nylon microfilament was introduced into the common carotid artery and the micro-clip released to allow advancement of the filament through the artery until the bulb-tip occluded the origin of the middle cerebral artery (MCA). This filament was left in place creating 1 hour of ischemia, and reperfusion was established by withdrawal of the filament. For sham groups, vessels were cleared of overlaying connective tissue (also performed in MCAO) without further manipulation. The wounds were closed using surgical sutures (6-0) and mice allowed to recover from anesthesia. Postoperative monitoring of eating, drinking and movement were performed at 4 and 24-hours following recovery.

Neurological testing. Neurological outcomes were evaluated at 4 and 24 hours after reperfusion using a 24-point scale. Briefly, mice were given positive scores (0-3) for each of the following parameters: 5 minutes of spontaneous activity, symmetry of movement and forelimbs (outstretching while tail is held), response to vibrissae contact, floor and beam walking, wire cage wall climbing, and reaction to touch on either side of the trunk.

hPMSCs isolation and culture. hPMSCs cells used in this study were isolated and were characterized using fluorescence-activated cell sorting (FACS) analysis or immunostaining. hPMSCs were CD73⁺, CD90⁺, CD44⁺and Oct-3/4⁺, and CD34⁻ and HLA-DR⁻. The 1° antibodies used included mouse anti-human CD73 (BD Biosciences; USA), mouse anti-human CD90 (BD Biosciences; USA), mouse anti-human CD34 (BD Biosciences; USA), mouse anti-human HLA-DR (BD Biosciences; USA), mouse anti-human CD44 (Santa Cruz Biotechnology; USA), and mouse anti-human Oct-3/4 (Santa Cruz Biotechnology; USA).

hPMSCs were cultured in Dulbecco's-Modified Eagles's Medium (DMEM; Fisher Scientific; USA) with 10% (w/v) fetal bovine serum (FBS; Gibco; USA) and 1% penicillin/streptomycin (Sigma; USA) and used at passage 3-10. At confluency, adherent hPMSCs cells were washed with 1×PBS-EDTA, detached with 0.25% trypsin (Sigma; USA) for 2 min, and subcultivated at a 1:3 split ratio.

IP Injection of hPMSCs. Trypsinized hPMSCs were washed with Hank's Balanced Salt Solution w/o Ca⁺⁺/Mg⁺⁺ twice, followed by 5 min centrifugation at 1500 rpm at 25° C. 5×10\hat{ }5 hPMSCs were resuspended in 500 ul HBSS solution w/o Ca⁺⁺/Mg⁺⁺, and then injected intraperitoneally (IP) into MCAO-treated mice at reperfusion.

Inhibition and induction of EVs formation. To investigate the effects of cholesterol depletion on hPMSC-enhanced MCAO outcomes, 10 mM methyl beta-cyclodextrin (MβCD), was added to medium as a non-toxic cholesterol sequestering agent for 48 h hours before harvesting the hPMSCs for use in MCAO studies. Conversely, to enrich hPMSCs cholesterol/lipid content, culture medium was supplemented with synthetic cholesterol (1:250 ratio) (Gibco; USA) and CD lipid concentrate (1:100 ratio) (Gibco; USA) and incubated for 72 h at 37° C., 5% CO₂ prior to cell harvesting.

Extracellular vesicle isolation. EVs were isolated. Briefly, culture media were collected from confluent hPMSCs 48 h after applying fresh medium. Unattached cells and debris were initially removed by centrifugation at 400×g for 10 min (4° C.) and supernatants transferred to fresh microcentrifuge tubes and re-centrifuged at 20,800×g for 90 min at 4° C. to pellet EVs. Supernatants were carefully aspirated, and EVs pellets washed twice by centrifugation using 4° C. PBS/1 mM phenylmethylsulfonyl fluoride (PMSF) and pelleted a final time at 20,800 g for 15 min (4° C.). Supernatants were aspirated, and EVs pellets injected intravenously (2×10\hat{ }6 in 100 ul HBSS, Sigma; USA) into mice or evaluated by flow cytometry analysis and western blotting.

Flow cytometry analysis. To evaluate hPMSC-released EVs by flow cytometry, freshly isolated EVs were resuspended in 100 μl Annexin V Binding Buffer (BD Biosciences, San Jose, Calif.) (10 mM HEPES, 140 mM NaCl, 2.5 mM CaCl₂) and incubated with 5 μl of Annexin V-FITC (BD Biosciences; USA) for 1 hour at 4° C. under low-light conditions. 900 ul of 1×“Binding Buffer” was added to each sample. These samples were immediately collected on a 4 laser ACEA NovoCyte Quanteon Flow cytometer and data analyzed using NovoExpress 1.2 software. EV flow cytometric analysis was calibrated using Megamix-Plus FSC and Megamix-Plus SSC beads.

Fluorescence activated cell sorting (FACS) of EVs. To study effects of PS negative-EVs in MCAO, the inventors isolated EVs from cholesterol-treated hPMSCs as described in the ‘Flow cytometry’ analysis section and then separated PS negative-EVs by (FACS) based on fluorescent labeling.

Cell localization using CytoID tracker. To track hPMSC in vivo, hPMSCs were first labelled using CytoID red long-term cell tracer kit (Enzo Life Science; USA) as described. Briefly, cells were trypsinized and labelled with labeling buffer, then incubated with 1 ml of 2×CytoID for 5 min. Staining was stopped by adding 2 ml of stop buffer. Cells were centrifuged at 400×g for 5 min, pellets resuspended in 10 ml of complete media (DMEM+10% FBS+1% P/S), transferred to a T75 flask, and incubated at 37° C. for at least 12 hours. CytoID-labelled hPMSCs were prepared and injected as described.

Laser Speckle measurement of blood flow. A Perimed Laser Speckle Imager (Pericam PSI HR; Sweden) was used to measure cerebral blood perfusion within the brains of different experimental groups. 24 hours after reperfusion, anesthesia was induced and maintained with 3% isoflurane, and mice were placed on a warm pad and of the coronal skin removed and perfusion recordings accomplished using a high-resolution Laser Speckle camera (Perimed Laser Speckle Imager) at a working distance of 10 cm. ‘Perfusion’ reflects total cerebral flow signal measured in selected tissue regions of interest. Measurements are expressed as perfusion units (PU), using a fixed scale (arbitrary units). Mice were decapitated, under deep anesthesia, and the brains TTC stained.

TTC staining of infarcted tissue. 24 hours after reperfusion, mice were deeply anesthetized with isoflurane and decapitated. The extent and severity of MCAO was evaluated after removal of the brain and staining of brain slices with 2,3,5-Triphenyltetrazolium chloride (TTC) (Sigma; USA) to measure tissue viability and infarct size. After dissection, the brain was immersed in cold PBS for 10 minutes and sliced into 2.0 mm-thick sections using an anatomical slicer. Brain slices were incubated in 2% TTC dissolved in PBS for 30 minutes at 37° C. Stained slices were then transferred to a glass plate wetted with PBS. The area of infarction in each brain slice was recorded with a digital Nikon 990 camera and measured using the Image-j program (NIH). Cumulative viable (red stained) regions were combined from each brain to generate a total brain tissue viability score for each mouse.

Evans blue vascular permeability evaluation. BBB disruption following MCAO/reperfusion was measured by quantitation of Evans blue transvascular leakage into the brains of mice at 24 hours. After MCAO, 2% Evans blue, an albumin complexing dye was dissolved in saline (4 mg/kg) and 100 ul injected through the femoral vein of mice under deep anesthesia and allowed to circulate for 20 min before sacrifice. To isolate plasma, 0.2 ml blood was collected from the left ventricle and centrifuged at 5000 rpm for 10 min. 10 ul plasma (supernatant) was added to 990 ul of 50% trichloroacetic acid (TCA; Sigma; USA), homogenized, sonicated and centrifuged at 10,000 rpm for 20 min. The circulating dye in the brain was cleared by perfusion of the mice with 15 ml cold PBS. To extract Evans blue from brain tissue, 2 ml of 50% TCA solution added to each brain in a scintillation tube, and the brain/TCA mixture homogenized and sonicated (amplitude 30, 10 W), and then centrifuged at 10,000 rpm for 20 min and finally diluted 3-fold with 100% ethanol. The amounts of Evans blue in both plasma and brain tissue were quantified at 620 nm excitation and 680 nm emission using Synergy H1 Hybrid Reader (BioTek; Vermont, USA). Evans blue leakage into brain tissue was then normalized to the amount of Evans blue in plasma.

Tissue preparation for immunohistochemistry and immunofluorescent staining. At 24 hours after reperfusion mice under deep anesthesia were cleared of blood with 15-20 ml of PBS. Brains were removed and post-fixed overnight in buffered 4% paraformaldehyde at 4° C. The brains were then sectioned into 30 pm sagittal slices and mounted on glass slides. For western blotting, mice were euthanized 24 hours after reperfusion, and brains dissected and frozen at −80 ° C. until use.

Immunohistochemistry staining. Following deparaffinization, rehydration and antigen retrieval with citrate buffer, 30 μm sagittal slices of brain tissue were incubated with 3% hydrogen peroxide (blocks endogenous peroxidase) and blocked with 1% bovine serum albumin (BSA; Sigma) and 4% normal goat serum in PBS-Triton (0.1%) for 1 hour at room temperature. The sections were incubated with rabbit anti-Iba-1 antibody (1:1000, Wako Pure Chemical Industries; USA) at 4° C. overnight and then treated with secondary biotinylated anti-rabbit IgG (1:200 in 1% BSA/PBST; Vector Laboratories; USA) for 2 hours at room temperature. The slices were then incubated with Avidin Biotin Complex (R.T.U) (LifeSpan BioSciences; USA) reagent for 1 hour at room temperature followed by peroxidase substrate (Vector Laboratories; USA). Peroxidase activity was visualized with 3-diaminobenzidine (DAB). Slides were then dehydrated with graded alcohols, cleared with xylene, and cover slipped.

Nissl staining. Tissue was fixed in 4% paraformaldehyde at room temperature for 24 h. Sagittal brain sections (30 μm) were mounted on glass slides. Nissl stains were performed as described. Briefly, samples were deparaffinized and rehydrated in decreasing ethanol concentrations. The slides were then processed for Nissl staining with thionin staining for ˜5 min at room temperature. Slides were dehydrated with graded alcohols, cleared with xylene, and cover slipped. Light microscopy (20× and 40× magnification) was used to visualize the Nissl stained sections.

IF staining of brain tissues. Expression of CD31 and Human nuclear marker (Hu-Nu) was assessed using fluorescently tagged secondary antibodies. Brain sections were treated as described. Briefly, after paraffinization and rehydration the sections were blocked with 1% BSA and 5% goat serum in PBS for 1 hour at room temperature, and then incubated in primary antibodies rabbit anti-CD31 (1:100; Abcam), mouse anti-human nuclear antibody (1:100; Millipore) overnight at 4° C. Following 4 washes in PBS for 10 min each, sections were stained with secondary antibodies included Alexa Fluor 488 goat anti-mouse (Life Technologies; USA), Alexa Fluor 647 goat anti-rabbit (Life Technologies; USA) for 2 hours at room temperature. Samples were then washed 4 times in PBS for 10 min each and mounted using DAPI/fluoroshield (Sigma; USA). Images were recorded using a Nikon video imaging system Eclipse E600FN (Nikon, Tokyo, Japan); and processed with NIH ImageJ software.

In vitro experiments. hCMEC-D3 culture. hCMEC-D3 cells (Dr. P. O. Couraud, INSERM) were cultured on collagen-coated plates using endothelial cell medium (EndoGRO; Millipore; USA) supplemented with MV complete culture media kit (Millipore; USA). When hCMEC-D3 cells reached 90% confluency, non-adherent dead cells were removed with 1×PBS-EDTA and adherent cells harvested using 0.25% trypsin (Sigma; USA), followed by centrifugation at 1500 rpm for 5 min at RT. Cells were then counted and plated at appropriate densities for each experiment.

Oxygen Glucose Deprivation Reperfusion (OGDR). 1×10\hat{ }5 hCMEC-D3 cells were plated and grown to 80% confluency. After changing the media to glucose free DMEM+10% (w/v) FBS+1% P/S, the cells were next incubated in a hypoxic chamber (1% O2) for 6, 12 or 16 h followed by 24 h reoxygenation in normal complete DMEM+10% FBS +1% P/S (5% O2).

Transwell Co-culture Model. In this model, hCMEC-D3 cells were plated on the bottom chamber of transwell plates (Corning, USA) in 2 ml of complete media and hPMSCs cultured on the upper surface of cell culture inserts with permeable membrane (3 μm pore size). In this contact-independent model, hPMSCs cannot migrate between compartments and do not directly interact with hCMEC-D3 cells.

Biotinylated gelatin/FITC avidin. To measure endothelial barrier function following OGDR, the inventors used the biotinylated gelatin/FITC avidin method. Biotinylated-gelatin stock solution was prepared by mixing Ez-link-biotin (Thermo Fischer; USA) in DMSO (5.7 mg/ml) (Sigma; USA) and then adding to gelatin (in NaHCO3 buffer (10 mg/ml), pH 8.3). Biotinylated gelatin stock solution was diluted in NaHCO₃ buffer (1:40 dilution) and added to 12-well plates, and incubated at 4° C. overnight. After removing the biotinylated gelatin solution, the wells were washed with PBS twice. hCMEC-D3 were plated at 2×10\hat{ }5 cells/well. A 1:50 dilution of FITC-avidin (Life Technologies-Molecular Probes; USA) was added directly to the media and incubated for 3 min at 37° C. under low light conditions. The media were removed, and the wells washed with 37° C. 1×PBS 2 times. Cells were fixed with 4% paraformaldehyde for 10 min at RT. Images were acquired using Nikon video imaging system Eclipse E600FN (Nikon, Tokyo, Japan); at 20×. Images were processed with NIH-ImageJ software.

Western blot analysis. After treatments, culture media were discarded and cells washed with 4° C. PBS. Laemmli sample buffer (Bio-Rad; USA) w/10% 2-mercaptoethanol was added to the cells. The cells were scraped and collected in microfuge tubes, sonicated at 50% power (15 s), boiled (95° C., 15 m) and stored at −80° C. 20 ul of protein was separated with SDS-PAGE and proteins then transferred to PVDF. Membranes were incubated with 1° antibodies overnight at 4° C.: rabbit anti-ZO-1 (1:500, Invitrogen), rabbit anti-α-claudin-1 (1:500, Invitrogen), rabbit anti-occludin (1:1000, Abcam), rabbit anti-α-catenin (1:1000, Abcam), rabbit anti-VE-cadherin (1:1000, Cell Signaling) and rabbit anti-α-tubulin (1:2000, Cell Signaling). Membranes were incubated with appropriate 2° antibodies at 25° C. for 2 h: goat anti-rabbit IgG-HRP antibodies (1:2500, Sigma). After signal detection using ChemiDocTM MP imaging system (Bio-Rad), results were analyzed with NIH Image-J software.

Immunofluorescence (IF) staining of hCMEC-D3. For immunofluorescent staining, hCMEC-D3 grown under normoxia/OGDR conditions were in some cases co-cultured with and w/o hPMSCs. Media were removed, and cells were washed with wash buffer (PBS+MgCl₂+CaCl₂+protease inhibitor). Cells were then fixed in ice-cold 4% paraformaldehyde for 10 min on ice, and permeabilized by 0.5% Triton X-100 in PBS for 5 min at room temperature. After two washes, the cells were blocked using 5% BSA+5% goat serum in PBS for 1 hour at room temperature. 1° antibodies (rabbit anti-α-catenin (1:100, Abcam) and rabbit anti-VE-cadherin (1:100, Cell Signaling) were diluted in wash buffer and incubated with cells overnight at 4° C. The antibodies were removed, and the cells rinsed with 4° C. wash buffer 2×. Cells were next incubated with fluorescently-conjugated 2° antibody (Alexa Fluor 488 goat anti-rabbit; Life Technologies; USA) for 1 h, and rinsed 2×. Hoechst (Thermo Scientific; USA) was added to the cells for 5 m and then washed out. After mounting the coverslips on glass slides, pictures were recorded using a Nikon video imaging system Eclipse E600FN (Nikon, Tokyo, Japan); using a 20× objective lens. Images were processed with NIH Image-J software.

Statistical analysis. Statistical analysis was performed using GraphPad Prism software. For all experiments, data are expressed as mean±standard error of the mean (SEM). The statistical significance of the differences between groups was calculated using Student's t-test, one-way ANOVA with Bonferroni post-hoc test or two-way ANOVA with Sidak's multiple comparisons tests where appropriate and indicated in the figure legend. A p-value<0.05 was considered statistically significant.

Enhanced Human Placenta-Derived Mesenchymal Stem Cells and Protection Against Stroke Injury in the MCAO Model of Stroke with Enhanced Stem Cells

Extracellular vesicles (EVs) derived from stem cells contribute at least in part to the ‘paracrine’ arm of SC-mediated benefit in stroke. EVs are biological vesicles released by cells and contain molecules that can modulate cell communication, repair and differentiation. Sterols, like cholesterol, are important structural components of EVs and also regulate their functional properties. To investigate the possible participation of hPMSC-derived EVs in the enhanced MCAO outcomes seen with hPMSC administration, the inventors blocked the formation/release of EVs from stem cells using 10 mM methyl-beta cyclodextrin (MμCD), as a non-toxic cholesterol chelating agent (FIG. 3A). The inventors observed unlike hPMSC treatment, MCAO mice injected with MβCD-treated hPMSCs (25.84±3.49) failed to show protection from MCAO injury (28.47±3.12, p=0.67, one-way ANOVA; FIG. 3B), similarly neurological deficits (4±1.77, p=0.73, one-way ANOVA; FIG. 3C) and cerebral blood flow (4.6±1.28, p=0.82, one-way ANOVA; FIG. 3D-F) interruption were comparable to the MCAO group (neurological score: 5.77±1.19 and blood flow: 3.94±1.37). Therefore, hPMSC-derived EVs appear to represent critical mediators of SC-based protection after stroke.

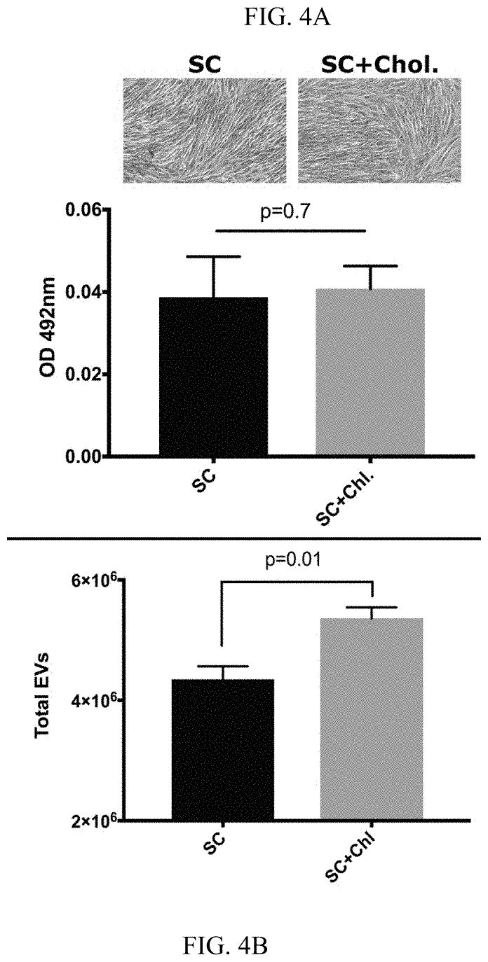

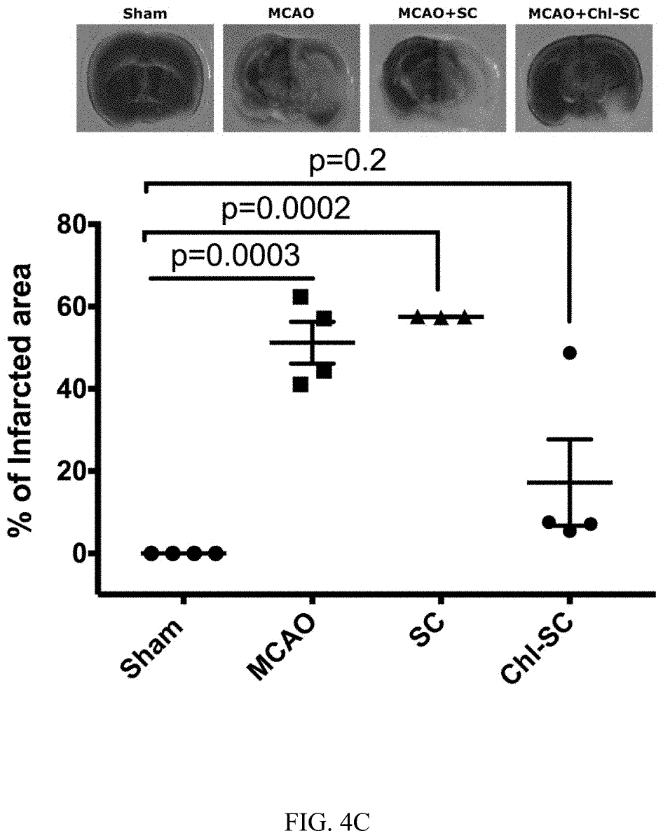

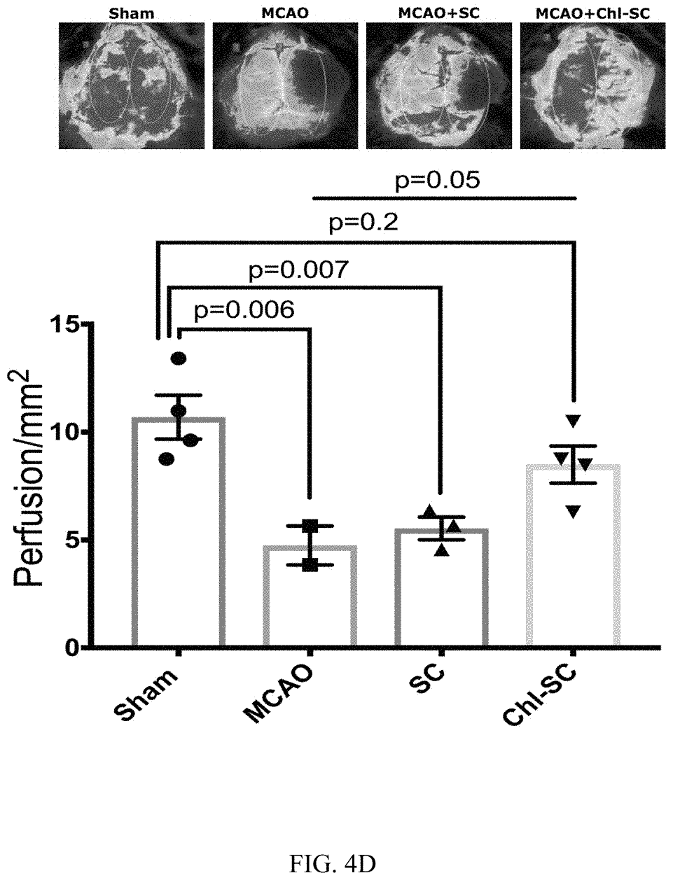

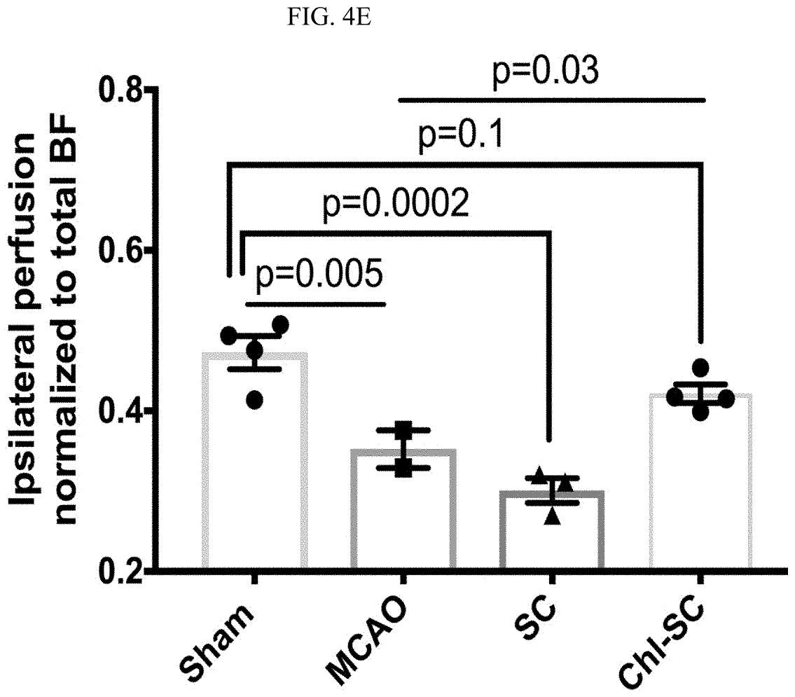

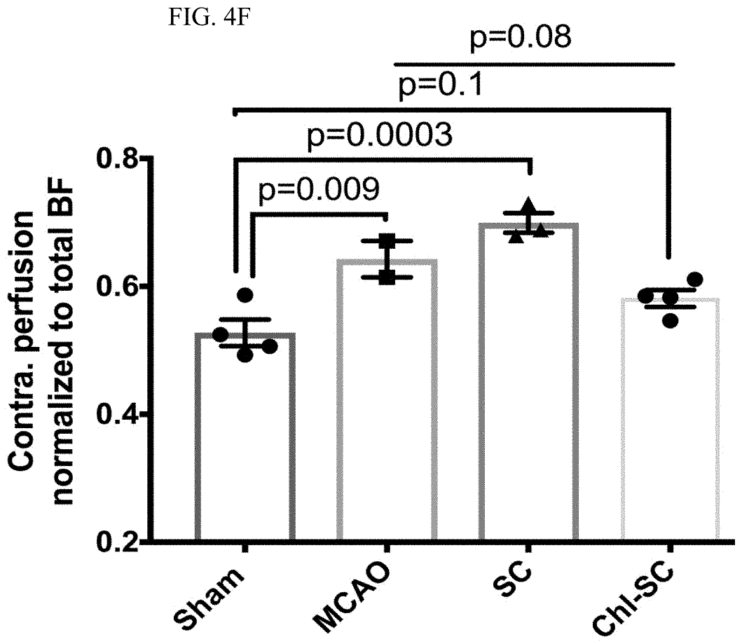

Cholesterol/lipid supplementation enhanced protective potential of hPMSCs in the MCAO model. To test how cholesterol might positively contribute to the formation and release of EVs, the inventors first treated hPMSC with the cholesterol-supplemented media and evaluated the cholesterol content of these cells using Oil Red O staining. There was no significant changes in staining for lipid and/or intracellular cholesterol (p=0.7, Student t-test; FIG. 4A), although flow cytometric analysis of EVs collected from the cholesterol-treated hPMSCs showed a significant increase in the number of EVs released from the cells after 72 hours treatment with cholesterol supplements (p=0.01, Student t-test; FIG. 4B), consistent with more EVs being formed upon addition of cholesterol (since cell cholesterol content was unchanged.).

Cholesterol/enhanced hPMSCs were prepared by culturing cells for 72 hours in 1×DMEM+10% FBS supplemented with synthetic cholesterol (1:250 dilution; Gibco, 12531-018) and Chemically Defined (CD) Lipid Concentrate (1:100 dilution; Gibco, 11905-031). These cells were tested for their ability to form extracellular vesicles using flow cytometry.

To evaluate the role of cholesterol in the protection provided by hPMSC, 1×10⁵ cholesterol-supplemented hPMSCs or ‘control’ hPMSCs (non-cholesterol supplemented cells) were injected IP into MCAO mice upon restoring blood flow (after 1 h ischemia). This reduced number of cholesterol-supplemented hPMSCs (20% of the number used previously (FIG. 1)) significantly prevented MCAO tissue injury (17.2±10.51, p=0.02, one-way ANOVA) while the same number of untreated hPMSC was insufficient to provide protection (57.5±0.06, p>0.9, one-way ANOVA; FIG. 4B). Behavioral performance (13±1.68, p=0.005; FIG. 4C) and blood perfusion (8.49±0.86, p=0.05; FIG. 4D-F) were also significantly improved in the 20%-dosed. cholesterol-supplemented hPMSCs group (one-way ANOVA). Therefore, cholesterol supplementation of hPMSC appears to significantly enhance their protective capacity in the MCAO model, apparently through the induction of EV formation and release.

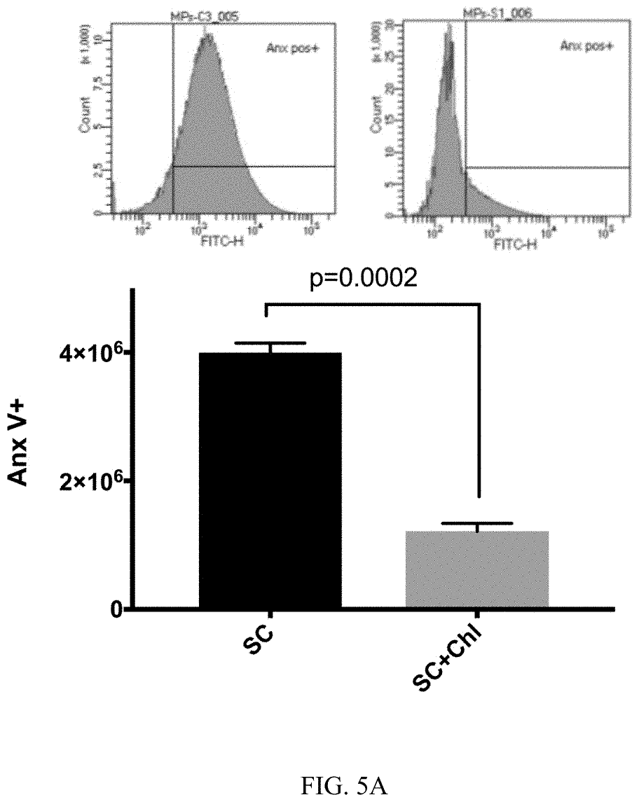

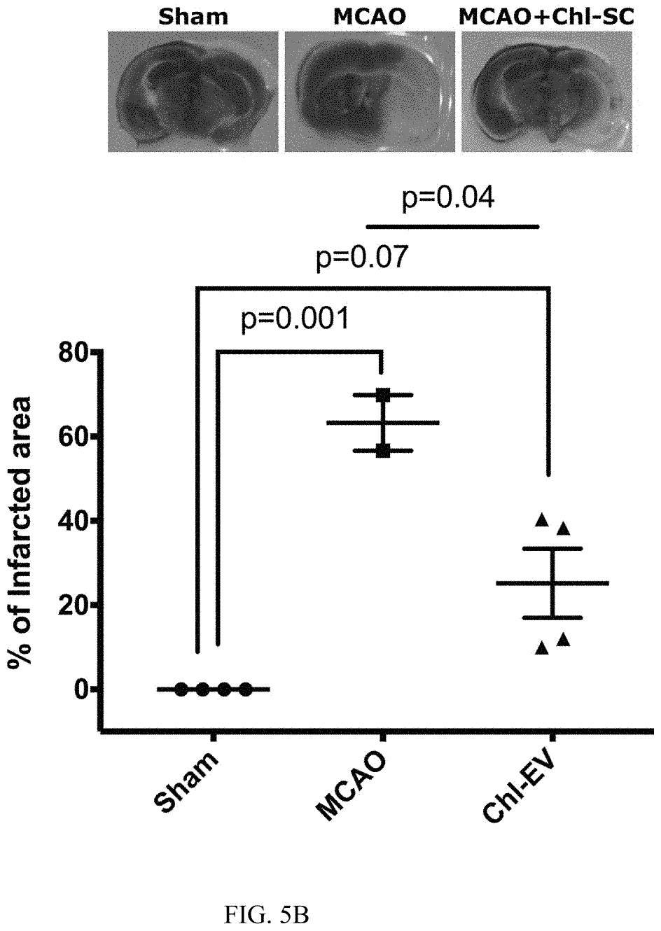

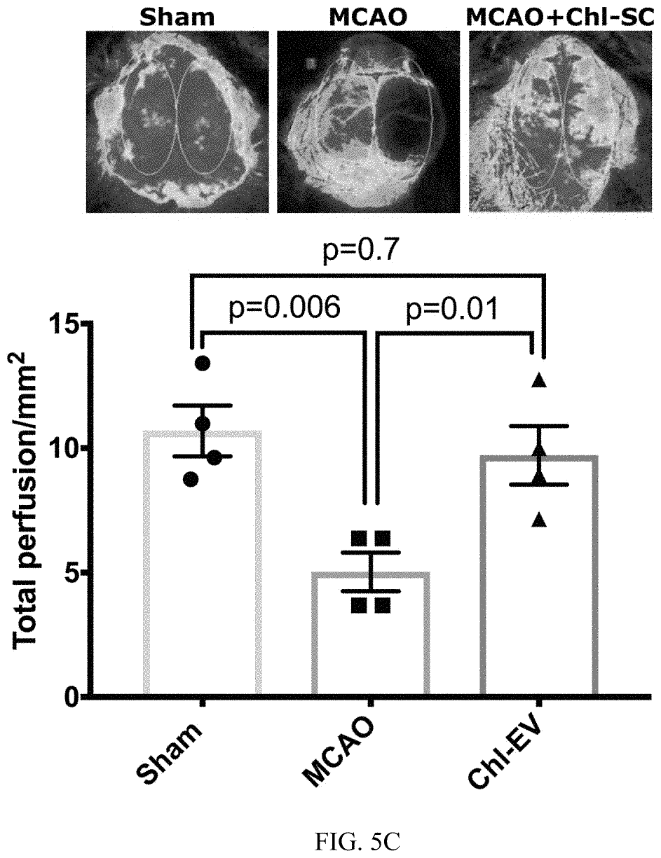

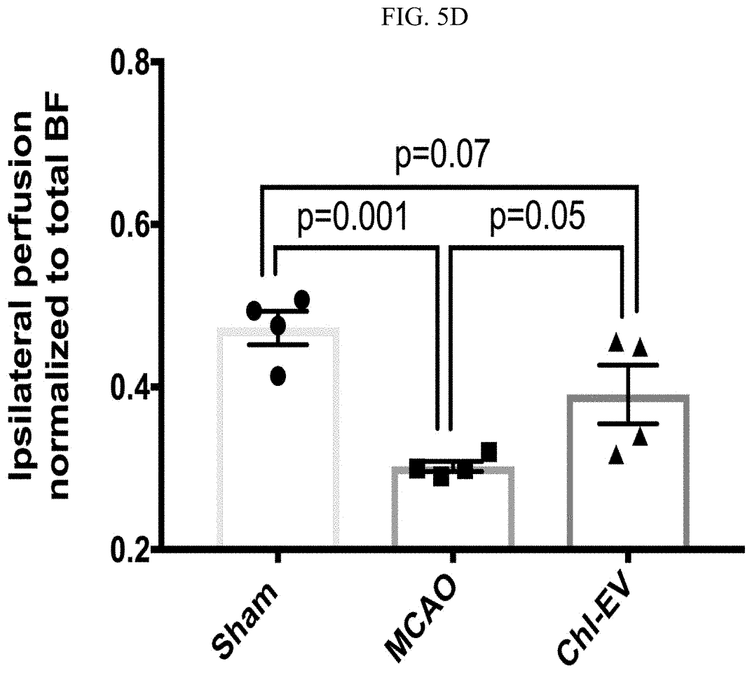

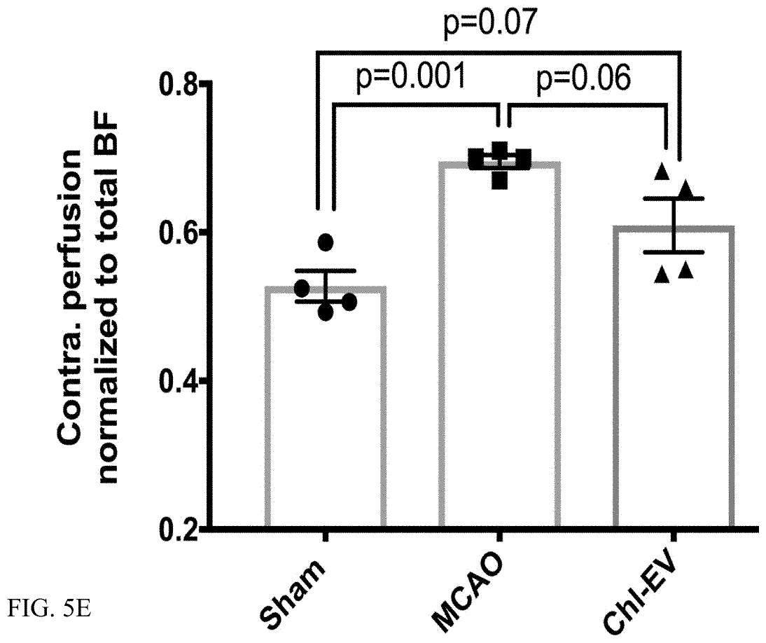

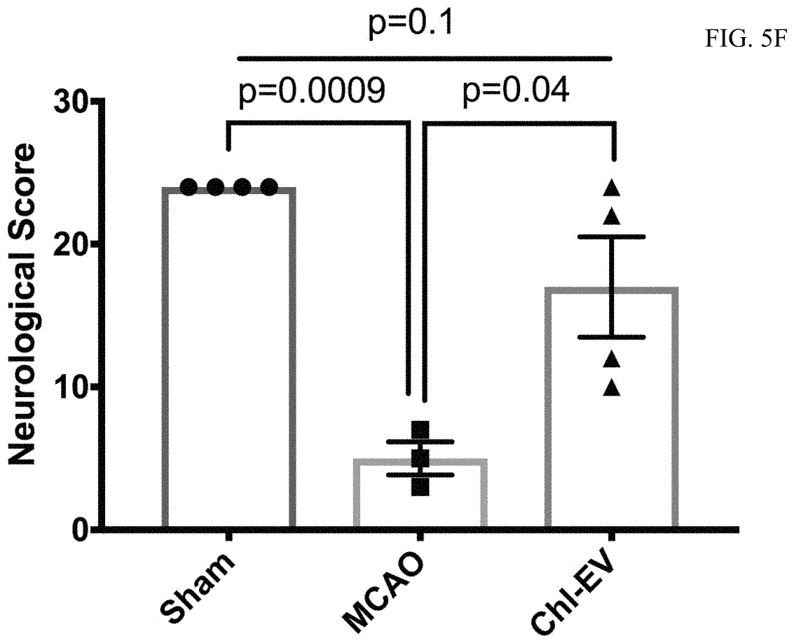

Surprisingly, flow cytometric analysis of EVs collected from cholesterol-hPMSCs revealed a significant decrease in annexin V positive EVs (p=0.0002, Student t-test; FIG. 5A), indicating that there is a net reduction in the expression of phosphatidylserine (PS) on the outer surface of EVs. PS is a pro-coagulant factor, which acts via recruitment of coagulation initiating proteases e.g., tissue factor (TF) and factor VII, that facilitate activation of factor X ultimately generating thrombin. Such an induction of the coagulation cascade may in part explain the inventors' observed decline in the survival of mice after IV injection of untreated hPMSCs or hPMSC-derived EVs. To identify whether cholesterol treatment of hPMSCs might overcome these IV injection-related complications, PS negative/cholesterol-supplemented hPMSCs derived EVs (2×10⁶ in 100 μl HBSS) (i.e. EVs that did not bind Annexin V) were sorted using FACS and were then injected IV into the MCAO mice. As shown in FIG. 5, this treatment produced a significant reduction in tissue injury from 58.68±4.43 in MCAO group to 25.16±8.21 in EVs derived from cholesterol-treated hPMSC (p=0.04, Student t-test; FIG. 5B) which was accompanied by improvement in blood perfusion (9.70±1.17, p=0.01, Student t-test; FIG. 5C-E) and behavioral performance (17±3.51, p=0.04; FIG. 5F).

By binding to cholesterol and disrupting lipid rafts MCD inhibited the formation and release of EVs, it is therefore very possible that stem cells cholesterol status is an important factor in hPMSC-dependent stroke protection. The inventors' studies demonstrated that cholesterol/lipid enrichment of hPMSC remarkably improved the effectiveness of hPMSCs in the inventors' MCAO model. For example, compared to the number of untreated stem cells which was found to protect the brain against MCAO (5×10⁵ cells; FIG. 1), a lower dose of untreated cells (1×10⁵ cells; FIG. 4) did not. In contrast, this same dose of cholesterol/lipid treated cells (1×10⁵ cells; FIG. 4) administered IP did significantly protect against MCAO induced injury (FIG. 4) indicating that cholesterol/lipid treated cells are at least 5 times more ‘potent’ (based on an efficacious ‘dose’).

In terms of scale, the dose of 5×10⁵ cells administered to a 27-30 g mouse suggests that an equivalent human IP dose (when adjusted for the mass scale of a 70 kg human) might approach 1×10⁹ stem cells for an IP dose. While this is feasible, it would be technically challenging, requiring a large commitment to produce cells and anticipates potentially ˜11,000 cm² cells for a single dose (˜60 T-175 flasks). However, if cholesterol/lipid supplemented cells were employed, a lower (potentially 20% or less) number of cells (2×10⁸) cells might be used (FIG. 4). Therefore, supplementation of hPMSCs with cholesterol/lipid could enhance technical scaling problem by reducing the number of required cells (from 60 to 12 T-175 flasks) per dose.

To the inventors' knowledge, although hPMSCs may be an abundant, non-immunogenic, and ethically ‘neutral’ source of EVs, purified EVs derived from hPMSCs have not yet been tested clinically for their protective effects in human stroke studies. Again, one of the major complications of IV administration of hPMSC (or hPMSC-derived EVs) has been activation of coagulation pathways, due to the presence of PS and TF on the surface of stem cells. With this in mind, the inventors found that cholesterol/lipid treatment of hPMSC significantly reduced the presentation of PS on the EVs outer surface (FIG. 5). Similarly, IV injection of sorted PS negative-EVs (2×10⁶ in 100 μl HBSS) collected from cholesterol-treated hPMSC was again found to significantly protect the brain against MCAO stroke injury (FIG. 5). Therefore, in further embodiments, future therapies for stroke will use either cholesterol/lipid stimulated hPMSCs, EVs from these cells, or both as potent therapeutics which stabilize cerebral perfusion and blood brain barrier and promote stable recovery from ischemic stress.

Enhanced human placenta-derived mesenchymal stem cells (hPMSCs) were prepared by culturing cells for 72 hours in 1×DMEM+ 10% FBS supplemented with synthetic cholesterol (1:250 dilution; Gibco, 12531-018) and CD lipid concentrate (1:100 dilution; Gibco, 11905-031). These enhanced stem cells were tested for their ability to form extracellular vesicles using flow cytometry. The addition of the 10% supplementation with synthetic cholesterol and CD lipid concentrate were the only differences between the culture preparation of the unmodified stem cells and the enhanced stem cells. The cells may be from term placental villous tissue.

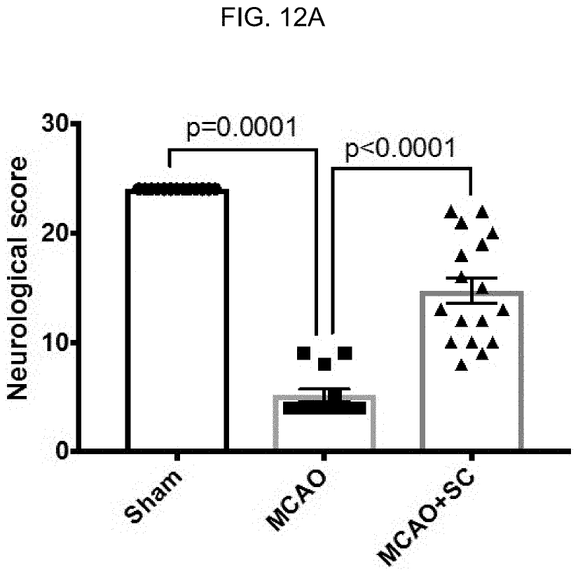

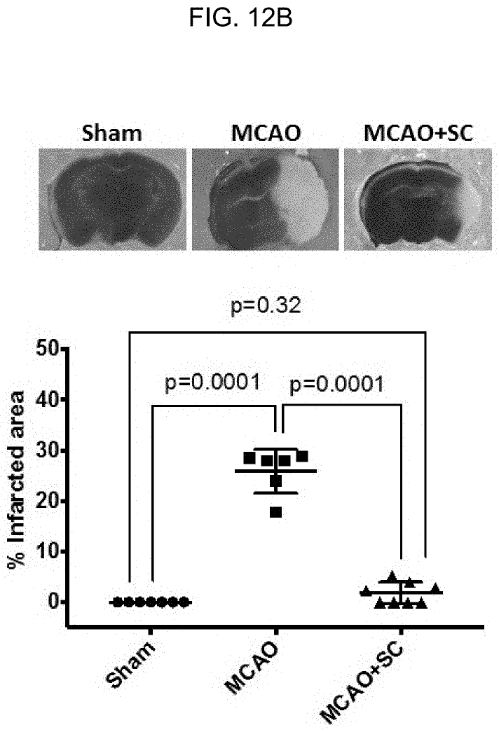

Turning to FIGS. 12A-12B, the inventors have shown that stem cells administered intraperitoneally strongly protects neurological score and infarct size against stroke injury (MCAO).

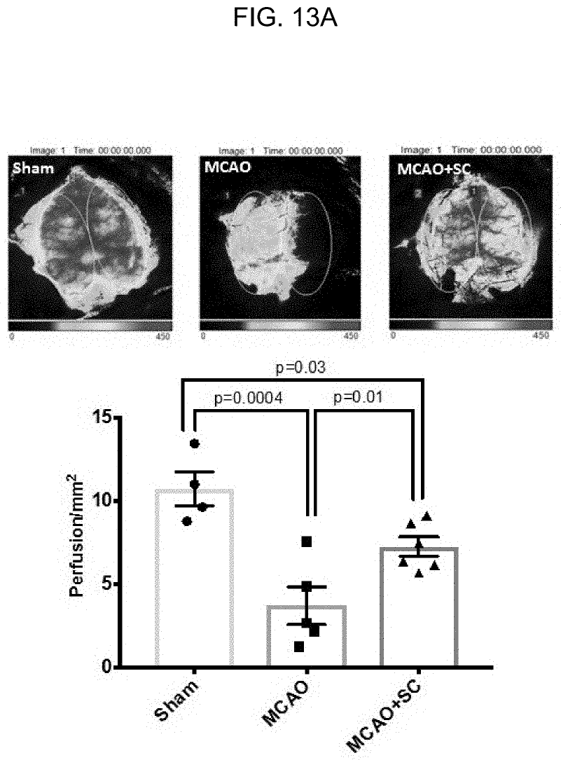

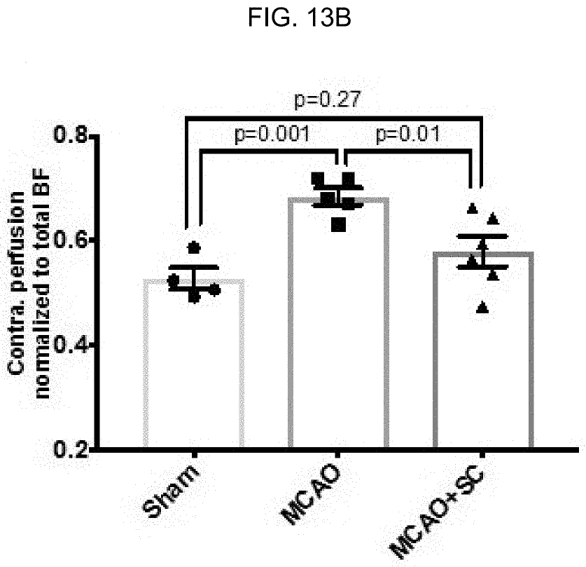

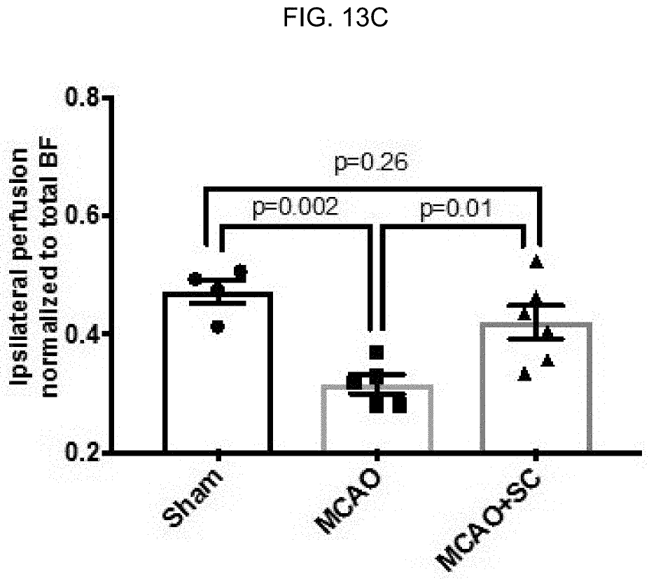

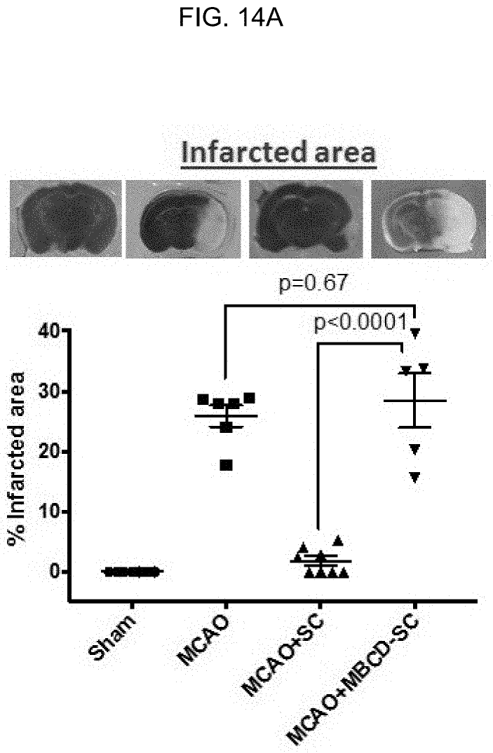

Turning to FIGS. 13A-13C, this protection by cells maintains brain blood flow and normalizes blood flow distribution between brain hemispheres. Important to the protection is the maintenance of blood flow to the brain at 24 hours, which is lost in normal stroke affected brains.

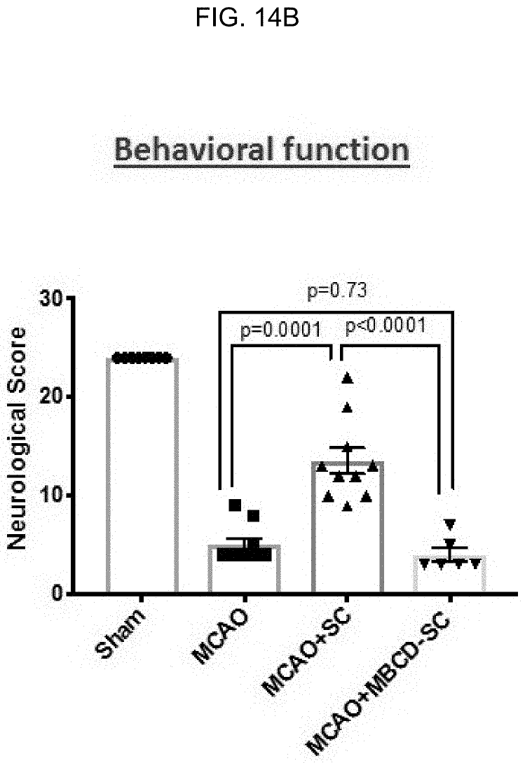

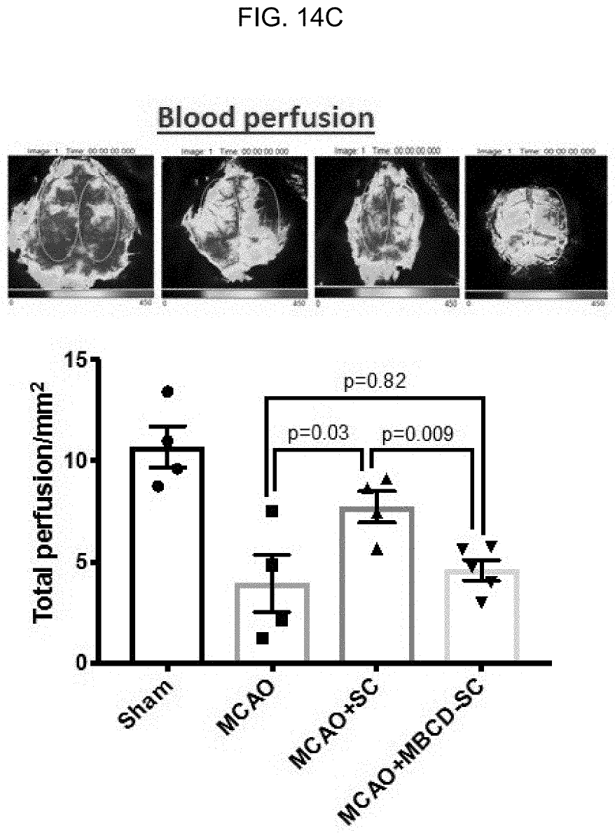

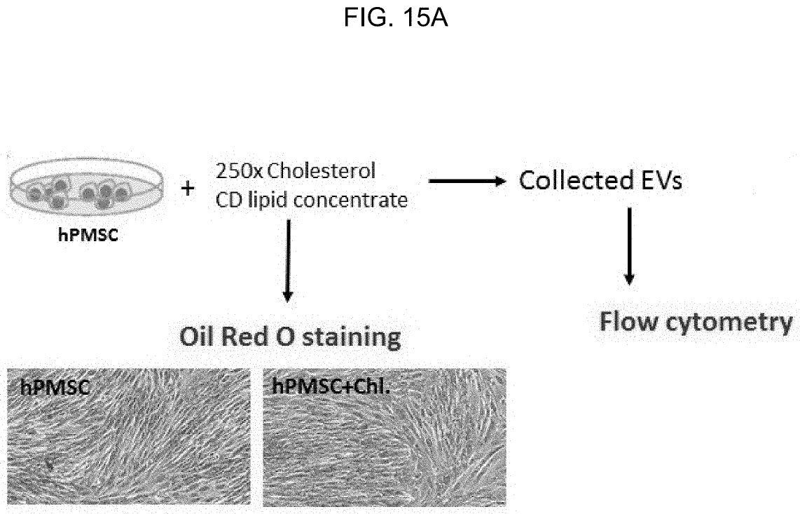

Turning to FIGS. 14A-14C, this form of protection is eliminated when cholesterol is removed from unmodified stem cells used in therapy using the cholesterol chelator MBCD. The depletion of stem cell cholesterol reduces protection for infarct area, neurological score and brain blood perfusion.

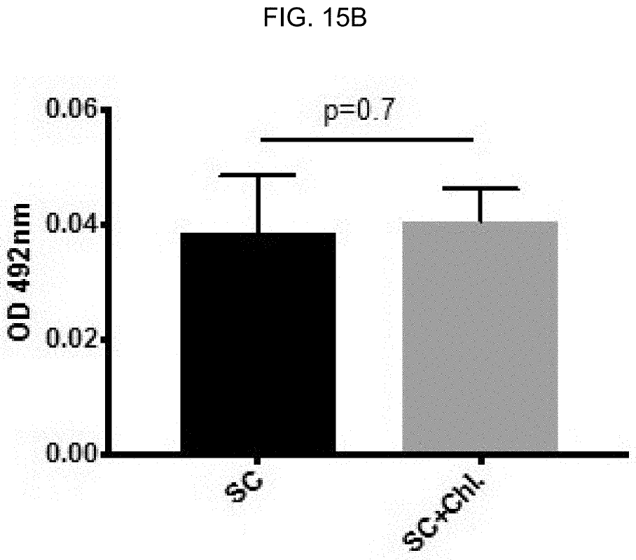

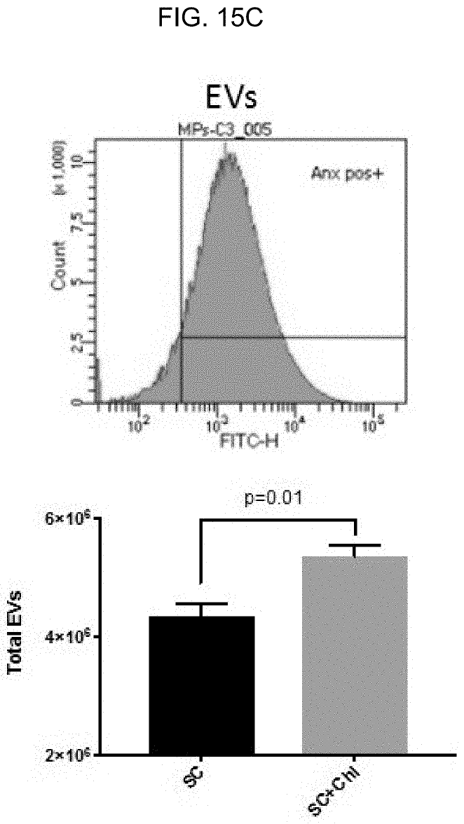

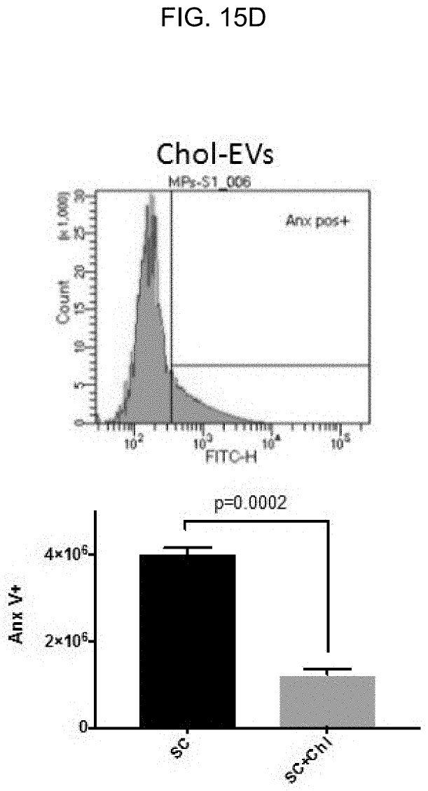

Turning to FIGS. 15A-15D, cholesterol and lipid treatment of stem cells increases total stem cell extracellular vesicle output and increases the proportion of Annexin-negative extracellular vesicles. Additionally, these Annexin-negative particles exhibit less phosphatidylserine on their surface which arguably enables the particles to more easily penetrate into tissues and have reduced influence on blood coagulation.

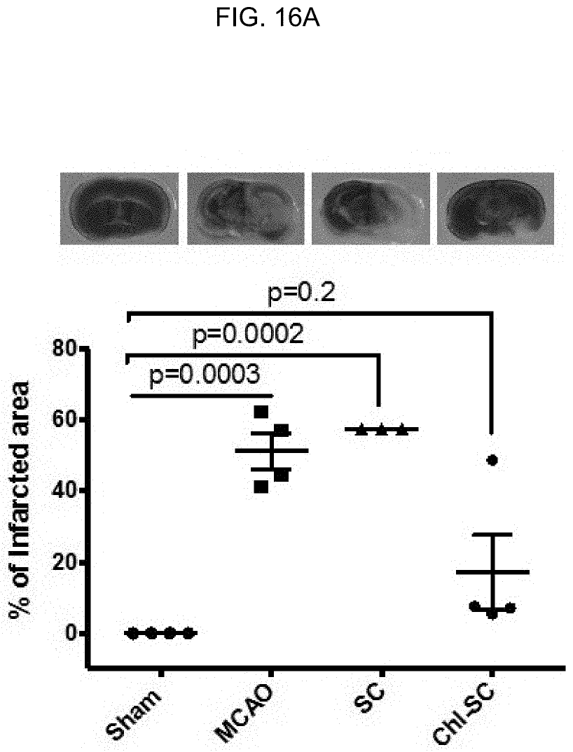

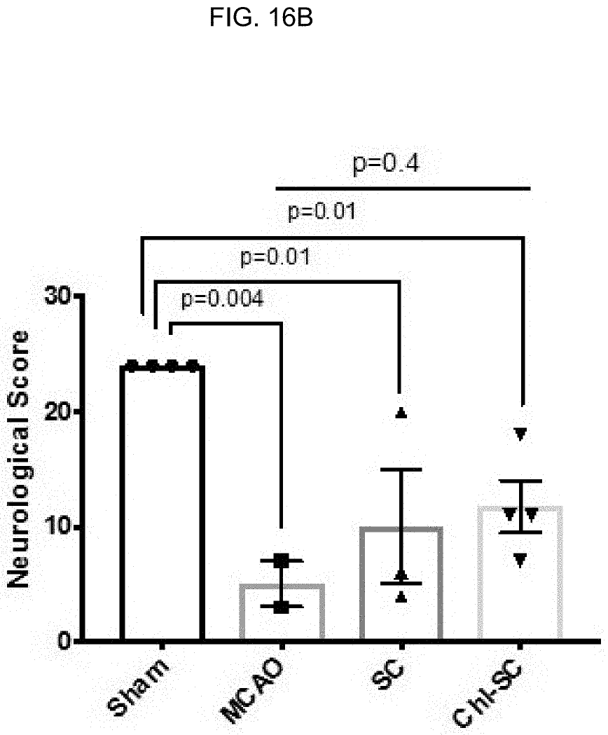

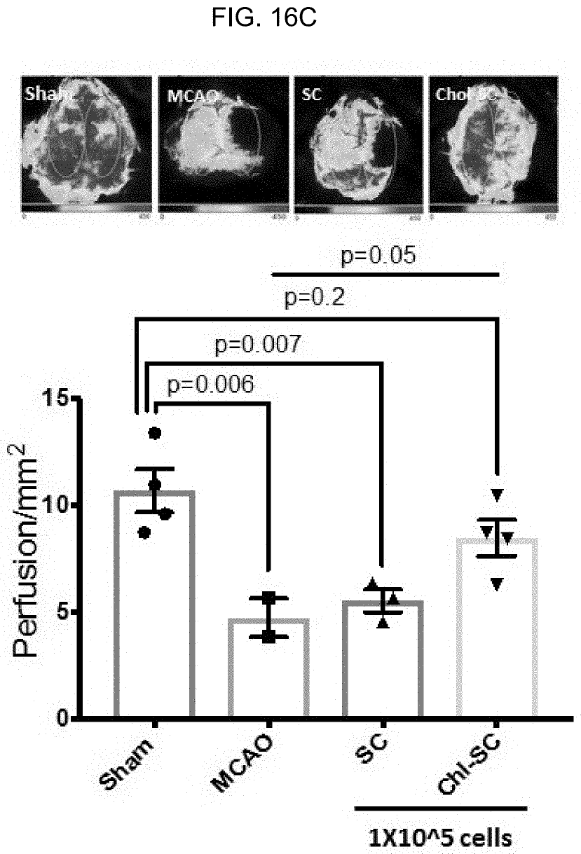

Turning to FIGS. 16A-16C, evidence for the five times enhanced protection against stroke of the enhanced stem cells involves using five times less enhanced stem cells to provide the same protection as unmodified stem cells. Stem cells are cholesterol and lipid supplemented to increase the cell exposure to cholesterol and lipids making the cells better able to form more exosome/microparticles. Importantly, because of the increased efficacy of the enhanced stem cells, the process makes it possible to use only 20% of the stem cells which normally provide complete protection against stroke injury in the middle cerebral artery occlusion model of stroke (MCAO). The inventors found that administration of 500,000 unmodified stem cells into the peritoneum was significantly protective in stroke models, but that 100,000 of these unmodified stem cells was not protective against stroke when given intraperitoneally. By comparison, when 100,000 cholesterol/lipid supplemented enhanced stem cells were administered, the inventors were able to obtain significant protection against stroke injury. Additionally, behavioral scores were also preserved when cholesterol/lipid supplemented/enhanced stem cells (at the lower dose of cells) are administered after stroke.

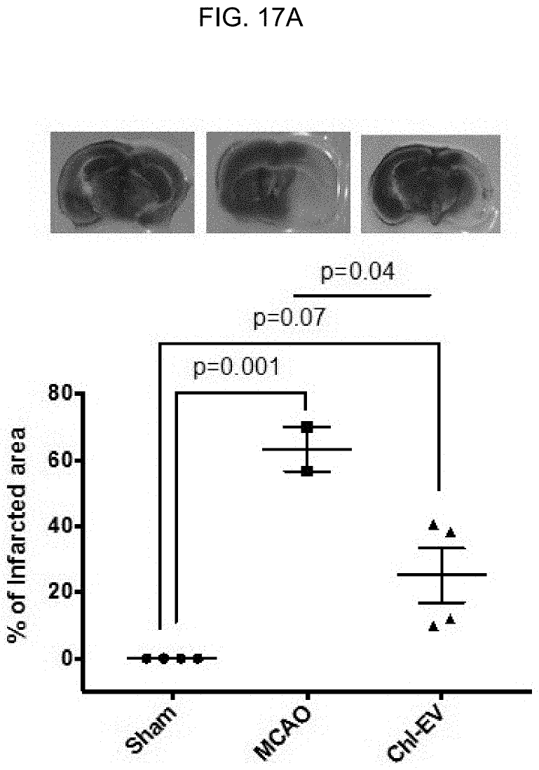

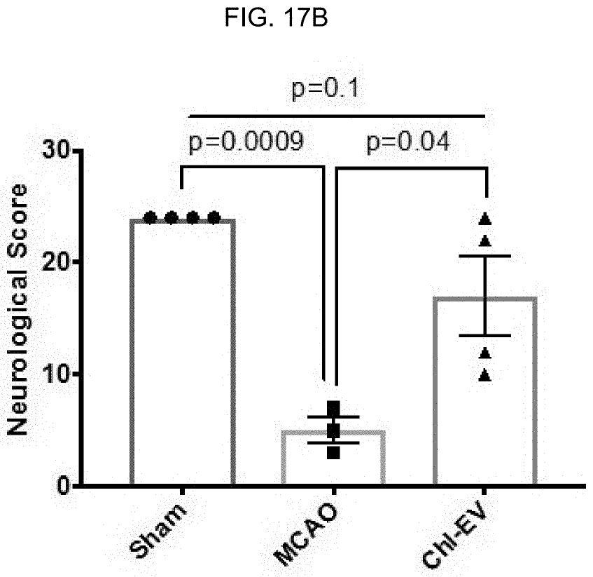

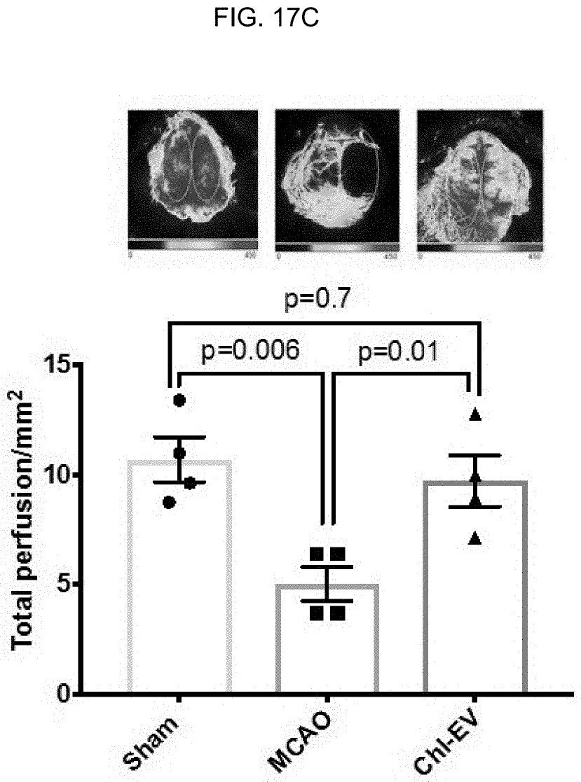

Turning to FIGS. 17A-17C, the enhanced exosomes, or exosomes from the enhanced stem cells treated in this fashion represent a cell-derived product which itself confers protection against reduced blood flow, neurological injury and infarct (damage) size with the brain after the brain has been stressed by ischemia-reperfusion. The exosomes/microparticles may be separated from the stem cells by filtration and centrifugation.

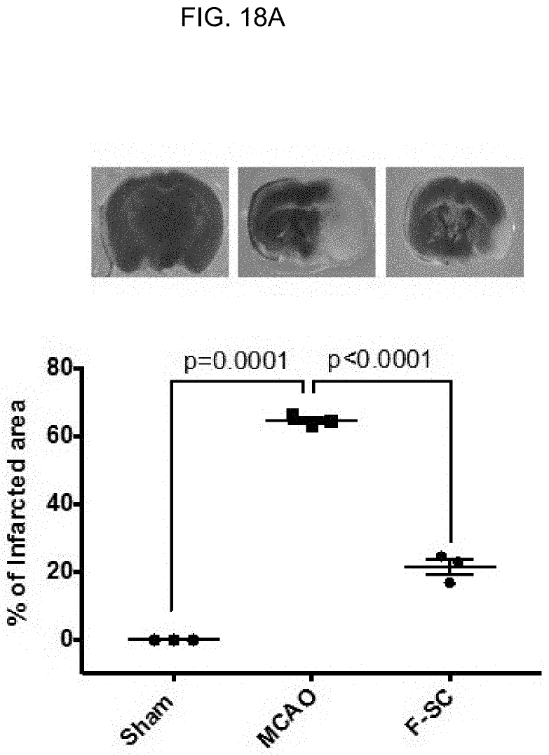

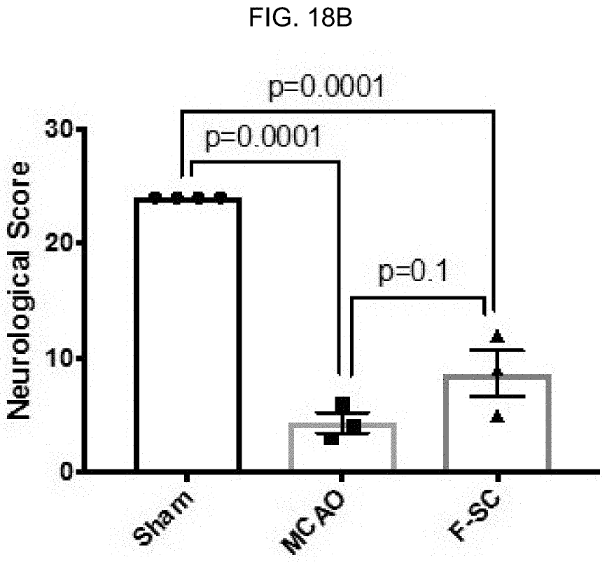

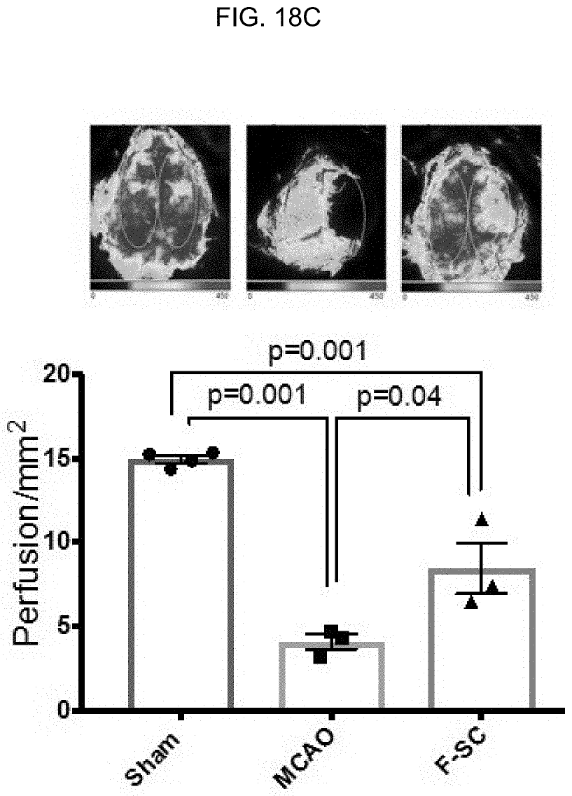

Turning to FIGS. 18A-18C, enhancing/activating the stem cells also allows the stem cells to be frozen for therapeutic administration at later time points with maintenance of the enhanced stem cells' beneficial properties, which is normally lost during the preservation/freezing process. The hPMSCs may be frozen in a freezing medium (1-5% dmso in fetal calf serum, e.g.) with cholesterol lipid supplementation (1-10%, e.g.) in such a process.

The inventors have evidence that cholesterol-lipid treatment may enable these enhanced stem cells and exosome/microparticles derived from them to be administered intravenously as well as intraperitoneally as demonstrated by use of intravenous enhanced stem cell vesicles after cholesterol treatment/enhancement of the stem cells. Additionally, stem cells prepared in this fashion (e.g., enhanced) are more stabilized for cryopreservation and retain effectiveness after freezing.

The enhanced stem cells and enhanced exosomes/microparticles are anticipated to be used, inter alia, to treat individuals who are seen at emergency departments for thrombotic strokes, particularly when tissue plasminogen activator (tPA) is administered. This treatment, when given after a stroke, has been shown to prevent the progressive brain tissue destruction, loss of function and behavioral and motor disturbances which often accompany stroke even when tPA is administered.

Enhancing the stem cells and the administration of the enhanced stem cells and/or the enhanced exosomes/microparticles are believed to be a completely novel treatments and products. These treatments and products do not appear to produce benefit by restituting cells lost as a result of injury, but rather preserve normal perfusion and tissue function at later time points. Because this treatment does not require cells to be given into the bloodstream, it is much safer and will not influence blood coagulation, which is a major and often lethal side-effect of stem cell administration.

The disclosure also describes the use of extracellular vesicles/microparticles, which are prepared from cholesterol/lipid treated/enhanced stem cells, as therapies for stroke which are cryostorable and which preserve blood flow, neurological function and tissue structure in the post-stroke affected brain. The extracellular vesicles/microparticles may be isolated from the stem cells and administered to the patient independent of the stem cells.

Kits containing normal and or enhanced stem cells for administration, and or normal and or enhanced exosomes and or microparticles for administration to patients is also conceived as part of the disclosed invention, including where the kits have an amount of normal and or enhanced stem cells and or normal and or enhanced exosomes and or microparticles for a single post stroke therapeutic administration.

The enhanced stem cells are conceived as part of the disclosed invention.

The exosomes/microparticles of the enhanced stem cells are conceived as part of the disclosed invention.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of creating enhanced stem cells from human Placenta Mesenchymal Stem Cells (hPMSCs) comprising:
culturing the hPMSCs with cholesterol for one of between 24 and 96 hours and between 48 and 72 hours.

2. A method of creating enhanced extracellular vesicles (EVs) from human Placenta Mesenchymal Stem Cells (hPMSCs) comprising:
culturing the hPMSCs with cholesterol for one of between 24 and 96 hours and between 48 and 72 hours, and
isolating the EVs from the hPMSCs.

3. A therapeutic comprising:
a plurality of extracellular vesicles (EVs),
wherein the EVs are derived from and isolated human placenta mesenchymal stem cells (hPMSCs), and
wherein the hPMSCs were cultured with cholesterol.
